(19) United States
(12) Patent Application Publication
(10) Pub. No.: US 2026/0198985 A1
(43) Pub. Date: Jul. 16, 2026

(54) INTESTINAL CATHETER DEVICE AND SYSTEM

(71) Applicant: Fractyl Health, Inc., Burlington, MA (US)

(72) Inventors: Sara A. Morneau, Burlington, MA (US); Michael A. Biasella, Burlington, MA (US); Jay Caplan, Burlington, MA (US); Harith Rajagopalan, Wellesley Hills, MA (US); Michelle Riches, Holden, MA (US); Andrew Hollett, Somerville, MA (US); Brendan Zarechian, Reading, MA (US); Kyle Dalbec, Boston, MA (US); Mark A. Manasas, Lexington, MA (US); Christopher J. Kadamus, West Roxbury, MA (US); Jeffrey Lesica, Holliston, MA (US); David A. Rezac, Westborough, MA (US); Richard Wisdom, Hyde Park, MA (US); J. Christopher Flaherty, Nottingham, NH (US); R. Maxwell Flaherty, Topsfield, MA (US)

(73) Assignee: Fractyl Health, Inc., Burlington, MA (US)

(21) Appl. No.: 19/445,384

(22) Filed: Jan. 9, 2026

Related U.S. Application Data
(63) Continuation of application No. 16/742,645, filed on Jan. 14, 2020, now abandoned.
Application No. 16/742,645 is a continuation of application No. PCT/US2018/042438, filed on Jul. 17, 2018.
(60) Provisional application No. 62/533,569, filed on Jul. 17, 2017.

(51) Int. Cl. A61B 18/06; A61B 18/00; A61B 18/02; A61B 18/04; A61M 25/00

(52) U.S. Cl. CPC A61B 18/06; A61B 18/02; A61B 2018/00005; A61B 2018/00041; A61B 2018/00166; A61B 2018/00244; A61B 2018/00291; A61B 2018/00494; A61B 2018/00577; A61B 2018/00982; A61B 2018/0212; A61B 2018/046; A61M 2025/004

(57) ABSTRACT

A system for ablating surface tissue of a patient is provided. The system includes a console and an ablation catheter fluidly attached to the console. The console includes an ablative fluid supply, a neutralizing fluid supply, an injectate fluid supply, a pump assembly, and a vacuum supply. The ablation catheter includes an expandable functional assembly, a tissue expansion subsystem for expanding sub-surface tissue in the intestine of the patient, and a tissue ablation subsystem for ablating surface tissue in the intestine of the patient. Methods of ablating surface tissue are also provided.

### CROSS-REFERENCE TO RELATED APPLICATIONS

This application is a continuation claiming the benefit of U.S. patent application Ser. No. 16/742,645, filed Jan. 14, 2020 and entitled “INTESTINAL CATHETER DEVICE AND SYSTEM,” which is hereby incorporated by reference herein in its entirety.

U.S. patent application Ser. No. 16/742,645 is a continuation claiming the benefit of PCT Patent Application PCT/US 18/42438, filed Jul. 17, 2018 and entitled “INTESTINAL CATHETER DEVICE AND SYSTEM,” which is hereby incorporated herein by reference in its entirety.

PCT Patent Application PCT/US18/42438 claims the benefit of U.S. Provisional Application No. 62/533,569 (Attorney Docket No. 41714-715.101, Client Docket No. MCT-025-PR1), filed Jul. 17, 2017, the entire content of which is incorporated herein by reference.

This application is related to U.S. patent application Ser. No. 13/945,138 (Attorney Docket No. 41714-703.301, Client Docket No. MCT-001-US), entitled “Devices and Methods for the Treatment of Tissue”, filed Jul. 18, 2013; U.S. patent application Ser. No. 14/470,503 (Attorney Docket No. 41714-704.301, Client Docket No. MCT-002-US), entitled “Heat Ablation Systems, Devices and Methods for the Treatment of Tissue”, filed Aug. 27, 2014; U.S. patent application Ser. No. 14/515,324 (Attorney Docket No. 41714-705.301, Client Docket No. MCT-003-US), entitled “Tissue Expansion Devices, Systems and Methods”, filed Oct. 15, 2014; U.S. patent application Ser. No. 14/609,332 (Attorney Docket No. 41714-706.301, Client Docket No. MCT-004-US), entitled “Electrical Energy Ablation Systems, Devices and Methods for the Treatment of Tissue”, filed Jan. 29, 2015; U.S. patent application Ser. No. 14/609,334 (Attorney Docket No. 41714-707.301, Client Docket No. MCT-005-US), entitled “Ablation Systems, Devices and Methods for the Treatment of Tissue”, filed Jan. 29, 2015; U.S. patent application Ser. No. 14/673,565 (Attorney Docket No. 41714-708.301, Client Docket No. MCT-009-US), entitled “Methods, Systems and Devices for Performing Multiple Treatments on a Patient”, filed Mar. 30, 2015; U.S. patent application Ser. No. 14/956,710 (Attorney Docket No. 41714-709.301, Client Docket No. MCT-013-US), entitled “Methods, Systems and Devices for Reducing the Luminal Surface Area of the Gastrointestinal Tract”, filed Dec. 2, 2015; U.S. patent application Ser. No. 14/917,243 (Attorney Docket No. 41714-710.301, Client Docket No. MCT-023-US), entitled “Systems, Methods and Devices for Treatment of Target Tissue”, filed Mar. 7, 2016; U.S. patent application Ser. No. 15/156,585 (Attorney Docket No. 41714-711.301, Client Docket No. MCT-024-US), entitled “Systems, Devices and Methods for the Creation of a Therapeutic Restriction in the Gastrointestinal Tract”, filed May 17, 2016; U.S. patent application Ser. No. 15/274,948 (Attorney Docket No. 41714-712.301, Client Docket No. MCT-027-US), entitled “Injectate Delivery Devices, Systems and Methods”, filed Sep. 23, 2016; U.S. patent application Ser. No. 15/274,764 (Attorney Docket No. 41714-714.501, Client Docket No. MCT-028-US-CIP1), entitled “Systems, Devices and Methods for Performing Medical Procedures in the Intestine”, filed Sep. 23, 2016; U.S. patent application Ser. No. 15/274,809 (Attorney Docket No. 41714-714.502, Client Docket No. MCT-028-US-CIP2), entitled “Systems, Devices and Methods for Performing Medical Procedures in the Intestine”, filed Sep. 23, 2016; U.S. patent application Ser. No. 15/406,572 (Attorney Docket No. 41714-713.301, Client Docket No. MCT-029-US), filed Jan. 13, 2017; U.S. Provisional Application No. 62/420,454 (Attorney Docket No. 41714-714.102, Client Docket No. MCT-028-PR2), entitled “Systems, Devices and Methods for Performing Medical Procedures in the Intestine”, filed Nov. 10, 2016; the entire contents of each of which are incorporated herein by reference in their entirety for all purposes.

### BACKGROUND OF THE INVENTION

### 1. Technical Field

The embodiments disclosed herein relate generally to systems, devices and methods for performing medical procedures in the intestine of a patient.

The field of gastrointestinal endoscopy has for many years focused on diagnostic and therapeutic techniques to observe, modify and remove tissues located in the digestive tract. For example, prior to a procedure to remove or otherwise modify tissue, a method referred to in the art as “lift and cut” involves the injection of saline or other biocompatible solution beneath the submucosa in an attempt to elevate and/or expand the submucosa, thereby changing the geometry to make it suitable for treatment, for example resection of tissue. In some cases, an injection catheter is used to deliver the fluid within the submucosal layer, which does not readily dissipate, throughout the target area, and once the target resection area has been elevated and/or expanded, the tissue can be treated.

However, the current devices, systems and methods for expanding submucosal and other tissue layers are cumbersome, inaccurate, and have a limited effected tissue area. Therefore, there is a need for improved devices, systems and methods for expanding submucosal and other tissue layers that provide simplified use, larger expansion areas, and reduced procedure time.

### SUMMARY OF THE INVENTION

According to one aspect of the invention, a system for ablating surface tissue comprises: a console and an ablation catheter. The console comprises: an ablative fluid supply for delivering ablative fluid; a neutralizing fluid supply for delivering neutralizing fluid; an injectate fluid supply for delivering injectate; a pump assembly for delivering and removing fluid from a device; a vacuum supply. The ablation catheter is fluidly attached to the console, and comprises: a distal portion; a flexible elongate shaft assembly comprising at least one shaft; an expandable functional assembly comprising at least one reservoir and positioned on the ablation catheter distal portion; a tissue expansion subsystem for expanding sub-surface tissue; and a tissue ablation subsystem for ablating surface tissue. The tissue expansion subsystem comprises: at least two tissue capture chambers, each tissue capture chamber positioned on the expandable functional assembly and configured to capture tissue when a vacuum is applied; at least two vacuum delivery conduits, each vacuum delivery conduit fluidly connected with one of the tissue capture chambers and for applying the vacuum to the tissue capture chamber; at least two injectate delivery elements, each injectate delivery element configured to deliver the injectate fluid to tissue captured by one of the tissue capture chambers; and at least two injectate delivery conduits, each injectate delivery conduit fluidly connected with one of the injectate delivery elements and for providing the injectate fluid to be delivered into tissue. The tissue ablation subsystem comprises: a first fluid delivery conduit fluidly connected with the at least one reservoir and for delivering the ablative fluid and the neutralizing fluid to the at least one reservoir; and a second fluid delivery conduit for removing fluid from the at least one reservoir.

In some embodiments, the elongate shaft assembly can comprise at least two lumens, and the ablation catheter can further comprise a second shaft comprising two lumens and a distal end operatively connected to a first tissue capture chamber and a third shaft comprising two lumens and a distal end operatively connected to a second tissue capture chamber, and the at least two vacuum delivery conduits can comprise a first vacuum delivery conduit comprising a proximal portion comprising a lumen of the first shaft and a distal portion comprising a first lumen of the second shaft, a second vacuum delivery conduit comprising a proximal portion comprising a lumen of the first shaft, and a distal portion comprising a first lumen of the third shaft. The ablation catheter can further comprise a manifold which fluidly connects proximal and distal portions of the first vacuum delivery conduit and proximal and distal portions of the second vacuum delivery conduit, and operably connects second lumen of second shaft with the lumen of the first shaft within which the first injectate delivery tube can be slidingly positioned and second lumen of a third shaft with the lumen of the first shaft within which the second injectate delivery tube can be slidingly positioned. The ablation catheter can further comprise a fourth shaft comprising two lumens and the at least two vacuum delivery conduits can further comprise a third vacuum delivery conduit comprising a proximal portion comprising a lumen of the first shaft and a distal portion comprising a first lumen of the fourth shaft. The at least two injectate delivery conduits can further comprise a third injectate delivery tube comprising a lumen and slidingly positioned within a lumen of the first shaft and a second lumen of the fourth shaft. The manifold fluidly connects proximal and distal portions of the third vacuum delivery conduit and operably connects second lumen of the fourth shaft with the lumen of the first shaft within which the third injectate delivery tube can be slidingly positioned.

In some embodiments, the at least one shaft comprises a twist, such as a counterclockwise twist.

In some embodiments, the system can be configured to perform at least two sequential injections of the injectate fluid, and each of the sequential injections can comprise a separation distance of between 1 cm and 2 cm from a previous injection.

In some embodiments, the system can be configured to perform multiple injections of the injectate fluid, and each of the injections can comprise a separation distance of at least 0.5 cm from a previous injection.

In some embodiments, the system can be configured to perform multiple injections of the injectate fluid, and each of the injections can comprise a separation distance that approximates half the length of the at least one reservoir from a previous injection.

In some embodiments, the system can be configured to reduce an amount of a fluid in the at least one reservoir during an injection of the injectate fluid into the tissue.

In some embodiments, the system can be configured to automatically apply a vacuum to a lumen of the intestine of the patient prior to a delivery of the injectate fluid into the tissue.

In some embodiments, the system can be configured to perform an assessment of a sub-surface tissue expansion prior to performing an ablation of the tissue. The system can be configured to automatically perform the assessment of the sub-surface tissue expansion. The system can be configured to perform the assessment of the sub-surface tissue expansion after a single sub-surface tissue expansion. The system can be configured to perform the assessment of the sub-surface tissue expansion after at least two sub-surface tissue expansions.

In some embodiments, the system can be configured to perform an assessment of a sub-surface tissue expansion using a camera of an endoscope.

In some embodiments, the system can be configured to perform an assessment of a sub-surface tissue expansion using an imaging device.

In some embodiments, the system can further include an image processing algorithm configured to perform at least a partial assessment of a sub-surface tissue expansion.

In some embodiments, an assessment of an expanded sub-surface tissue can comprise a qualitative assessment performed by a clinician and/or a quantitative assessment performed automatically and/or semi-automatically by the system. An inadequate expansion can suggest the expansion of a new area of sub-surface tissue. An inadequate expansion can suggest termination of a procedure. The assessments can be configured to identify a patient with a condition selected from the group consisting of: an active infection in the duodenum; a history of an infection, such as tuberculosis; a malignancy that can cause a duodenal injury; and combinations thereof. The assessments can be configured to identify a significant fibrosis and/or a significant scar at a target location. The system can be configured to perform an ablation if the sub-surface tissue expansion is adequate.

In some embodiments, the system can be configured to perform an ablation without repositioning the expandable functional assembly subsequent an expansion of a sub-surface tissue.

In some embodiments, the system can be configured to treat at least three axial segments of a patient's duodenal mucosal tissue.

In some embodiments, the console can be configured to provide one or more fluids to the expandable functional assembly, and the one or more fluids can comprise a fluid selected from the group consisting of: an inflation fluid; an ablative fluid; a neutralizing fluid; and combinations thereof. The console can be configured to provide the one or more fluids to the expandable functional assembly at a flow rate of at least 2 mL/sec or at least 5 mL/sec. The console can be configured to provide the one or more fluids to the expandable functional assembly at a flow rate of approximately 9.5 mL/sec. The console can be configured to provide the one or more fluids to the expandable functional assembly at a flow rate not greater than 30 mL/sec.

In some embodiments, the console can further comprise a manifold. The manifold can be constructed and arranged to fluidly combine one or more conduits. The manifold can be constructed and arranged to divide one or more conduits. The manifold can include one or more valves configured to control the flow of a fluid within a conduit. The manifold can include one or more sensors configured to provide a signal related to a parameter of a fluid within a conduit.

The one or more sensors can comprise a temperature and/or pressure sensor. The parameter can comprise a temperature and/or pressure of the fluid.

In some embodiments, the console can further comprise an inflation fluid supply for delivering inflation fluid. The inflation fluid supply can be configured to provide the inflation fluid to the expandable functional assembly. The inflation fluid supply can be configured to provide the inflation fluid to the expandable functional assembly at a flow rate of at least 2 mL/sec or at least 5 mL/sec. The inflation fluid supply can be configured to provide the inflation fluid to the expandable functional assembly at a flow rate of approximately 9.5 mL/sec. The inflation fluid supply can be configured to provide the inflation fluid to the expandable functional assembly at a flow rate not greater than 30 mL/sec.

In some embodiments, the ablative fluid supply can be configured to provide the ablative fluid to the expandable functional assembly. The ablative fluid supply can be configured to provide the ablative fluid to the expandable functional assembly at a flow rate of at least 2 mL/sec or at least 5 mL/sec. The ablative fluid supply can be configured to provide the ablative fluid to the expandable functional assembly at a flow rate of approximately 9.5 mL/sec. The ablative fluid supply can be configured to provide the ablative fluid to the expandable functional assembly at a flow rate not greater than 30 mL/sec.

In some embodiments, the neutralizing fluid supply can be configured to provide neutralizing fluid to the expandable functional assembly. The neutralizing fluid supply can be configured to provide neutralizing fluid to the expandable functional assembly at a flow rate of at least 2 mL/sec or at least 5 mL/sec. The neutralizing fluid supply can be configured to provide neutralizing fluid to the expandable functional assembly at a flow rate of approximately 9.5 mL/sec. The neutralizing fluid supply can be configured to provide neutralizing fluid to the expandable functional assembly at a flow rate not greater than 30 mL/sec. The neutralizing fluid can be provided to the at least one reservoir during a sub-surface tissue expansion. The neutralizing fluid can be configured to pre-cool and/or pre-warm a tissue proximate the at least one reservoir prior to an ablation of the tissue. The neutralizing fluid can be configured to post-cool and/or post-warm a tissue shortly after an ablation of the tissue, and the neutralizing fluid can be configured to limit the effects of a heat ablation and/or a cryogenic ablation. The neutralizing fluid can be configured to reduce time in a previous and/or subsequent ablation step.

In some embodiments, the injectate fluid supply can comprise a pump. The pump can comprise a syringe pump configured to drive two or more syringes simultaneously.

In some embodiments, the injectate fluid supply can comprise two or more injectate delivery elements. The two or more injectate delivery elements are each configured to deliver a fluid simultaneously at a rate of at least 10 mL/min, at least 12.5 mL/min, at least 15 mL/min, at least 20 mL/min, at least 40 mL/min, or at least 60 mL/min. The injectate fluid supply can be configured to deliver a volume of a fluid to the two or more injectate delivery elements. The volume of fluid can be between 2 mL and 20 mL. The volume of fluid can be delivered in a time period of less than 60 seconds, less than 40 seconds, less than 30 seconds, less than 20 seconds, less than 10 seconds, or less than 5 seconds.

In some embodiments, the injectate fluid supply can be configured to deliver a fluid at a pressure of at least 40 psi, at least 75 psi, at least 100 psi, at least 200 psi, or at least 300 psi.

In some embodiments, the injectate fluid supply can be configured to expand an axial segment of sub-surface tissue, the axial segment of sub-surface tissue can comprise a length of at least 0.25 cm, at least 0.5 cm, or at least 0.75 cm.

In some embodiments, the injectate fluid supply can be configured to expand a sub-surface tissue layer to a thickness of at least 250 μm. The injectate fluid supply can be configured to expand the sub-surface tissue layer to a thickness of approximately 400 μm.

In some embodiments, the injectate fluid can comprise an agent configured to cause a necrosis of the tissue.

In some embodiments, the injectate fluid can comprise a warming fluid and/or a cooling fluid, the warming fluid and/or cooling fluid can be delivered onto and/or into the tissue.

In some embodiments, the injectate fluid can comprise a neutralizing fluid configured to limit, stop, and/or at least reduce the ablation of the tissue.

In some embodiments, the console can be configured to deliver the injectate fluid to the at least two injectate delivery elements at a flow rate of at least 10 mL/min, at least 50 mL/min, or at least 100 mL/min.

In some embodiments, the console can be configured to deliver a full volume of injectate fluid to one of the at least two injectate delivery elements within a time period not greater than 2 minutes, not greater than 1 minute, or not greater than 30 seconds.

In some embodiments, the injectate fluid can comprise a visualizable material. The visualizable material can comprise India Ink. The visualizable material can comprise Indigo Carmine. The visualizable material can be configured to be visualized by a camera of an endoscope and/or the ablation catheter, and the camera can provide one or more images for an assessment of a sub-surface tissue expansion. The visualizable material can be used to determine a proper volume of the injectate delivered. The determination of the proper volume can include monitoring the pressure of the at least one reservoir and/or the volume of the injectate fluid within the at least one reservoir. The visualizable material can be used to determine a sufficient tissue expansion. The determination of the sufficient tissue expansion can include analyzing the expanded tissue to identify one or more regions of adherent tissue, and the adherent tissue can comprise scarred and/or fibrotic tissue.

In some embodiments, the injectate fluid can comprise a radiopaque material. The radiopaque material can be configured to be visualized by an imaging device, and the imaging device can provide one or more images for an assessment of a sub-surface tissue expansion. The imaging device can comprise a fluoroscope or other X-ray imaging device.

In some embodiments, the injectate fluid can comprise an ultrasonically reflectable material. The ultrasonically reflectable material can be configured to be visualized by an imaging device, and the imaging device can provide one or more images for an assessment of a sub-surface tissue expansion. The imaging device can comprise an ultrasound imaging device.

In some embodiments, the vacuum supply can be configured to provide a vacuum pressure of between −2 psi and −14.7 psi. The vacuum supply can be configured to provide a vacuum pressure of between 31 4 psi and −14.7 psi. The vacuum supply can be configured to provide a vacuum pressure of between −6 psi and −12.5 psi.

In some embodiments, the vacuum supply can be configured to provide a vacuum pressure, and the vacuum supply can further comprise at least one sensor to monitor the vacuum pressure.

In some embodiments, the system can further comprise a fluid removal pump. The fluid removal pump can be configured to remove fluid from the at least one reservoir.

In some embodiments, the system can further comprise an insufflation supply configured to deliver and/or remove fluid from the intestine of the patient.

In some embodiments, the system can further comprise a functional fluid supply configured to deliver and/or remove functional fluid to and/or from the ablation catheter.

In some embodiments, the ablation catheter can include one or more visualization markers configured to allow a visualized guidance of a translation of the ablation catheter.

In some embodiments, the ablation catheter can include one or more visualization markers configured to allow a visualized guidance of a rotation of the ablation catheter.

In some embodiments, the ablation catheter can be configured to translate a pre-determined distance. The pre-determined translation distance can be at least 0.3 cm or at least 0.6 cm.

In some embodiments, the ablation catheter can comprise a handle portion. The handle portion can include a tactical thermal status indicator. The handle portion can include an inflow conduit positioned proximate to a thermally conductive housing portion, and a user of the ablation catheter can detect a relative temperature of a fluid within the inflow conduit. The handle portion can include one or more functional elements comprising a heating and/or cooling transducer configured to provide real-time information of a temperature to a user.

In some embodiments, the ablation catheter can be configured to expand two or more axial segments of the sub-surface tissue, and the ablation catheter can be further configured to ablate the expanded two or more axial segments. A cumulative length of the ablated two or more axial segments can be greater than a cumulative length of the expanded two or more axial segments.

In some embodiments, the injectate delivery element can be positioned above a source of vacuum provided to the tissue capture chamber.

In some embodiments, the injectate delivery element can comprise a needle. The needle can comprise a diameter of between 16 gauge and 34 gauge. The needle can comprise a diameter of 27 gauge. The needle can comprise a diameter of 29 gauge. The needle can comprise a bevel angle of between at least 5° and not greater than 80°. The bevel angle can be not greater than 45°. The bevel angle can be approximately 10°. The needle can be configured to penetrate tissue at the time a vacuum is applied to the tissue capture chamber. The needle can be not advanced within the tissue capture chamber. The needle can be advanced within the tissue capture chamber. The needle can deliver a fluid into the tissue capture by the tissue capture chamber. The tissue capture chamber can be configured to slidingly receive the needle, and the needle can comprise a diameter of at least 29 gauge, or at least 27 gauge.

In some embodiments, the injectate delivery element can be configured to be advanced a distance of at least 2.5 mm, at least 3.5 mm, or at least 4.5 mm.

In some embodiments, the tissue capture chamber can comprise a width of least 0.010″, at least 0.040″, or at least 0.060″. The width can be not greater than 0.35″. The width can be not greater than 0.25″.

In some embodiments, the tissue capture chamber can comprise a length of least 0.010″, least 0.040″, or at least 0.060″. The length can be not greater than 0.9″, not greater than 0.7″, or not greater than 0.5″.

In some embodiments, the tissue capture chamber can comprise a depth of at least 300 μm, at least 500 μm, or at least 700 μm. The depth can be not greater than 1500 μm.

In some embodiments, the tissue capture chamber can comprise a metal and/or a material with a relatively high thermal conductance.

In some embodiments, the injectate delivery element can comprise a fluid jet. The fluid jet can be configured to deliver a fluid through the surface of and into the tissue captured within the tissue capture chamber.

In some embodiments, a single injectate delivery element can be configured to deliver an injection comprising a volume of injectate fluid of at least 1 mL, at least 2 mL, at least 5 mL, or at least 8 mL. The single injectate delivery element can be configured to deliver an injection comprising a volume of injectate fluid not greater than 20 mL or not greater than 15 mL. The single injectate delivery element can be configured to deliver an injection comprising a volume of injectate fluid of approximately 10 mL.

In some embodiments, the at least two injectate delivery elements can be configured to deliver injections comprising a cumulative volume of injectate fluid of at least 3 mL, at least 6 mL, at least 15 mL, or at least 24 mL. The at least two injectate delivery elements can be configured to deliver injections comprising a cumulative volume of injectate fluid not greater than 60 mL, not greater than 45 mL, or not greater than 30 mL. The at least two injectate delivery elements can be configured to deliver injections comprising a cumulative volume of injectate fluid of approximately 30 mL.

In some embodiments, the at least two injectate delivery elements can be configured to deliver a volume of injectate fluid to expand the tissue to a thickness of at least 250 μm. The at least two injectate delivery elements can be configured to deliver a volume of injectate fluid to expand the tissue to a thickness of approximately 400 μm.

In some embodiments, the expandable functional assembly can be at least partially collapsed prior to a translation of the ablation catheter.

In some embodiments, each of the at least two tissue capture chambers include an opening. The opening can comprise a width less than or equal to 2.0 mm. The opening can comprise a width of approximately 1.5 mm. The opening can comprise a width of approximately 1 mm. The opening can comprise a length less than or equal to 5.0 mm. The opening can comprise a length of approximately 4.0 mm. The opening can comprise one or more projections. The one or more projections can be configured to prevent and/or minimize damage to the tissue by restricting the depth at which the tissue can descend into the opening upon the application of a vacuum or other negative pressure.

In some embodiments, the expandable functional assembly can be constructed and arranged to expand a sub-surface tissue at a single axial location, and the expanded sub-surface tissue can comprise an expanded tissue periphery that can be sufficiently sized to surround an ablation periphery.

In some embodiments, the expandable functional assembly can be constructed and arranged to expand a sub-surface tissue at two or more axial locations, and the cumulative expanded sub-surface tissue can comprise an expanded tissue periphery that can be not sufficiently sized to surround an ablation periphery.

In some embodiments, the expandable functional assembly can be configured to at least partially collapse prior to and/or during an assessment of a sub-surface tissue expansion. The at least partially collapsed expandable functional assembly can be configured to provide an increased view of the expanded sub-surface tissue. The at least partially collapsed expandable functional assembly can be configured to allow an advancement of an endoscope toward and potentially into an axial segment of the expanded sub-surface tissue to provide a closer view of the tissue.

In some embodiments, the expandable functional assembly can comprise a tissue contacting length of between 0.5 cm and 4.0 cm. The tissue contacting length can be between 1.5 cm and 3.3 cm. The tissue contacting length can be approximately 2 cm.

In some embodiments, the expandable functional assembly can further comprise a manifold. The manifold can be constructed and arranged to fluidly combine one or more conduits. The manifold can be constructed and arranged to divide one or more conduits. The manifold can include one or more valves configured to control the flow of a fluid within a conduit. The manifold can include one or more sensors configured to provide a signal related to a parameter of a fluid within a conduit. The one or more sensors can comprise a temperature and/or pressure sensor. The parameter can comprise a temperature and/or pressure of the fluid.

In some embodiments, the expandable functional assembly includes a functional element comprising a heat-generating transducer, such as a heat-generating transducer comprising at least one electrode configured to deliver RF energy to generate heat (e.g. to heat fluid contained within the functional assembly).

In some embodiments, the expandable functional assembly includes a functional element comprising a cooling transducer, (e.g. to cool fluid contained within the functional assembly).

In some embodiments, each of the at least two tissue capture chambers can comprise a first lumen and a second lumen, and the second lumen can be positioned above the first lumen.

The first lumen can comprise a crescent shaped cross section geometry and the second lumen can comprise a cylindrical geometry. The crescent shaped geometry of the first lumen can relatively surround the cylindrical geometry of the second lumen.

In some embodiments, the at least one reservoir can comprise a single reservoir that can circumferentially surround the elongate shaft assembly.

In some embodiments, the at least one reservoir can comprise multiple reservoirs in a lobed arrangement.

In some embodiments, the at least one reservoir can comprise at least one inner reservoir surrounded by at least one outer reservoir.

In some embodiments, the at least one reservoir can comprise one or more balloons that circumferentially surround the at least one shaft in a linear arrangement.

In some embodiments, the at least one reservoir can comprise multiple partially circumferential balloons that surround the at least one shaft in a radial arrangement.

In some embodiments, the at least one reservoir can comprise one or more inner balloons surrounded by one or more outer balloons.

In some embodiments, the at least one reservoir can comprise a balloon. The balloon can be configured to expand to a diameter less than or equal to 35 mm, less than or equal to 30 mm, or less than or equal to 25 mm. The balloon can comprise a wall thickness of at least 0.00025″, at least 0.00035″, or at least 0.00050″. The balloon can comprise a wall thickness of approximately 0.00075″.

In some embodiments, the at least one reservoir can comprise one or more portions comprising a non-compliant material and one or more portions comprising a compliant material.

In some embodiments, the expandable functional assembly can comprise at least two reservoirs. The first of the at least two reservoirs can comprise at least a portion comprising a non-compliant material, and the second of the at least two reservoirs can comprise at least a portion comprising a compliant material.

In some embodiments, the elongate shaft assembly can comprise at least six lumens, and the at least one tissue capture chamber can comprise a first tissue capture chamber and a second tissue capture chamber, and a first pair of lumens are in fluid communication with the first tissue capture chamber, a second pair of lumens are in fluid communication with the second tissue capture chamber, and a third pair of lumens are in fluid communication with the at least one reservoir of the expandable functional assembly. The at least two injectate delivery elements can comprise a first injectate delivery element and a second injectate delivery element, and the first pair of lumens can comprise a vacuum lumen and a lumen that can slidingly receive a first tube attached to the first injectate delivery element, the second pair of lumens can comprise a vacuum lumen and a lumen that can slidingly receive a second tube attached to the second injectate delivery element, and the third pair of lumens can comprise a fluid delivery lumen that can deliver a fluid to the at least one reservoir and a fluid removal lumen that can remove a fluid from the at least one reservoir. The elongate shaft assembly can comprise a single tube comprising the at least six lumens. The elongate shaft assembly can comprise at least eight lumens, and the at least one tissue capture chamber can further comprise a third tissue capture chamber and a fourth pair of lumens that are in fluid communication with the third tissue capture chamber. The at least two injectate delivery elements can further comprise a third first injectate delivery element and the fourth pair of lumens can comprise a vacuum lumen and a lumen that can slidingly receive a third tube attached to the third injectate delivery element. The elongate shaft assembly can further comprise a fluid recirculation conduit comprising a wall and surrounding a fluid transport tube including a lumen, and the third pair of lumens can comprise the lumen of the fluid transport tube and the space between the wall of the fluid recirculation conduit and the fluid transport tube. The lumen of the fluid transport tube can deliver a fluid to the at least one reservoir, and the space between the wall of the fluid recirculation conduit and the fluid transport tube can remove a fluid from the at least one reservoir. The lumen of the fluid transport tube can remove a fluid from the at least one reservoir, and the space between the wall of the fluid recirculation conduit and the fluid transport tube can deliver a fluid to the at least one reservoir. The elongate shaft assembly can comprise a guidewire lumen and/or at least one insufflation lumen. The at least one insufflation lumen can comprise a first insufflation lumen and a second insufflation lumen. The shaft assembly can comprise an insufflation lumen that can terminate in one or more openings. Each of the one or more openings can be configured to perform only one of insufflation or desufflation.

In some embodiments, the elongate shaft assembly can comprise a first lumen for delivering inflation fluid, ablation fluid and/or neutralizing fluid to the at least one reservoir, and a second, separate lumen that can remove a fluid from the at least one reservoir.

In some embodiments, the elongate shaft assembly can further comprise a manifold. The manifold can be constructed and arranged to fluidly combine one or more conduits. The manifold can be constructed and arranged to divide one or more conduits. The manifold can include one or more valves configured to control the flow of a fluid within a conduit. The manifold can include one or more sensors configured to provide a signal related to a parameter of a fluid within a conduit. The one or more sensors can comprise a temperature and/or pressure sensor. The parameter can comprise a temperature and/or pressure of the fluid.

In some embodiments, the system can further comprise an endoscope.

In some embodiments, the system can further comprise a guidewire over which the ablation catheter can be translated.

In some embodiments, the system can further comprise an imaging device. The imaging device can comprise a device selected from the group consisting of: endoscope camera; visible light camera; infrared camera; X-ray imager; fluoroscope; CT scanner; MRI; PET scanner; ultrasound imaging device; molecular imaging device; and combinations thereof.

In some embodiments, the system can further comprise an agent. The system can be configured to deliver the agent to the intestine of the patient. The agent can comprise a material selected from the group consisting of: anti-peristaltic agent, such as L-menthol; glucagon; buscopan; hyoscine; somatostatin; diabetic medication; analgesic agent; opioid agent; chemotherapeutic agent; hormone; and combinations thereof. The agent can comprise cells delivered into the intestine of the patient. The agent can comprise a mucolytic agent configured to remove a mucus from the surface of the tissue.

In some embodiments, the system can further comprise a handle assembly. The handle assembly can include multiple guide tubes. Each of the multiple guide tubes can terminate within a corresponding lumen, and the guide tubes can terminate less than 1.25″, less than 1″, or less than 0.13″ from a proximal end of the shaft. The handle assembly can further comprise a manifold. The manifold can be constructed and arranged to fluidly combine one or more conduits. The manifold can be constructed and arranged to divide one or more conduits. The manifold can include one or more valves configured to control the flow of a fluid within a conduit. The manifold can include one or more sensors configured to provide a signal related to a parameter of a fluid within a conduit. The one or more sensors can comprise a temperature and/or pressure sensor. The parameter can comprise a temperature and/or pressure of the fluid.

In some embodiments, the system can further comprise a marker configured to be placed in the intestine in reference to non-target tissue.

In some embodiments, the system can be configured to treat and/or diagnose a patient disease or disorder selected from the group consisting of: Type 2 diabetes; Type 1 diabetes; “Double Diabetes”; gestational diabetes; hyperglycemia; pre-diabetes; impaired glucose tolerance; insulin resistance; non-alcoholic fatty liver disease (NAFLD); non-alcoholic steatohepatitis (NASH); obesity; obesity-related disorder; polycystic ovarian syndrome (PCOS); hypertriglyceridemia; hypercholesterolemia; psoriasis; GERD; coronary artery disease (e.g. as a secondary prevention); stroke; TIA; cognitive decline; dementia; Alzheimer's Disease; neuropathy; diabetic nephropathy; retinopathy; heart disease; diabetic heart disease; heart failure; or diabetic heart failure. The system can be configured to treat and/or diagnose two or more of a patient disease or disorder selected from the group consisting of: Type 2 diabetes; Type 1 diabetes; “Double Diabetes”; gestational diabetes; hyperglycemia; pre-diabetes; impaired glucose tolerance; insulin resistance; non-alcoholic fatty liver disease (NAFLD); non-alcoholic steatohepatitis (NASH); obesity; obesity-related disorder; polycystic ovarian syndrome (PCOS); hypertriglyceridemia; hypercholesterolemia; psoriasis; GERD; coronary artery disease (e.g. as a secondary prevention); stroke; TIA; cognitive decline; dementia; Alzheimer's Disease; neuropathy; diabetic nephropathy; retinopathy; heart disease; diabetic heart disease; heart failure; diabetic heart failure; and combinations thereof. The system can be configured to treat and/or diagnose two or more of a patient disease or disorder selected from the group consisting of: diabetes; insulin resistance; non-alcoholic fatty liver disease (NAFLD); non-alcoholic steatohepatitis (NASH); and polycystic ovarian syndrome (PCOS).

In some embodiments, the system can be configured to treat and/or diagnose a patient selected based on a level of a patient parameter selected from the group consisting of: body mass index (BMI) level; waist circumference; HbAlc level; fasting glucose; insulin resistance; liver fibrosis; cholesterol or triglyceride level; duration of years exhibiting type 2 diabetes; fasting insulin, fasting C-peptide or C-Peptide stimulation in response to a meal; age; and combinations thereof.

### BRIEF DESCRIPTION OF THE DRAWINGS

The foregoing and other objects, features and advantages of embodiments of the present inventive concepts will be apparent from the more particular description of preferred embodiments, as illustrated in the accompanying drawings in which like reference characters refer to the same or like elements. The drawings are not necessarily to scale, emphasis instead being placed upon illustrating the principles of the preferred embodiments.

FIG. 1 is a schematic view of a system for performing a medical procedure in the intestine of a patient, consistent with the present inventive concepts.

FIG. 1A is a schematic view of a system for performing a medical procedure in the intestine of a patient, consistent with the present inventive concepts.

FIG. 1B is a flow chart of a method for performing a medical procedure in the intestine of a patient, consistent with the present inventive concepts.

FIG. 1C is an anatomical view of a segment of the duodenum in which two tissue expansions are performed to provide a safety margin for a single ablation, consistent with the present inventive concepts.

FIGS. 2 and 2A are perspective views of an example handle portion of a catheter, consistent with the present inventive concepts.

FIGS. 3, 3A and 3B are a perspective view, and two side sectional views of a fluid delivery element control assembly of a catheter, consistent with the present inventive concepts.

FIGS. 4 and 4A are perspective and sectional views, respectively, of an inflation control assembly of a catheter, consistent with the present inventive concepts.

FIGS. 5, 5A and 5B are a perspective view and two magnified sectional views of an interface between a handle assembly and a shaft assembly of a catheter, consistent with the present inventive concepts.

FIG. 6 is a perspective view of a shaft assembly of a catheter, consistent with the present inventive concepts.

FIGS. 7A-7B illustrate the distal portion of a catheter including a functional assembly, consistent with the present inventive concepts.

FIGS. 8A-8D are sectional views of a shaft assembly and manifold assembly of a catheter, consistent with the present inventive concepts.

FIGS. 9A-9B are top and sectional views, respectively, of a tissue capture chamber of a catheter, consistent with the present inventive concepts.

FIGS. 10A-10B are top and sectional views, respectively, of a tissue capture chamber of a catheter, consistent with the present inventive concepts.

FIG. 11 is a side sectional view of the distal portion of a catheter, consistent with the present inventive concepts.

FIGS. 11A-11D are various sectional views of the catheter of FIG. 11, consistent with the present inventive concepts.

FIGS. 12 and 12A-12D are perspective, top, side, side sectional, and sectional views, respectively, of a tissue capture chamber of a catheter, consistent with the present inventive concepts.

FIGS. 13A-16C are top, perspective, and side views (A-C of each, respectively) of a tissue capture chamber of a catheter, consistent with the present inventive concepts.

FIGS. 17A-17C are side sectional views of a tissue capture chamber and a fluid delivery element of a catheter, advanced to different positions, consistent with the present inventive concepts.

FIG. 18 is a perspective view of a skive tool, consistent with the present inventive concepts.

FIG. 19 is a perspective view of a twisting tool, consistent with the present inventive concepts.

FIG. 20 is a flow chart of a method for preparing a multi lumen shaft of a catheter, consistent with the present inventive concepts.

FIG. 21 is a perspective view of a handle of a catheter, including a tactile thermal status indicator, consistent with the present inventive concepts.

### DETAILED DESCRIPTION OF THE DRAWINGS

The terminology used herein is for the purpose of describing particular embodiments and is not intended to be limiting of the inventive concepts. Furthermore, embodiments of the present inventive concepts may include several novel features, no single one of which is solely responsible for its desirable attributes or which is essential to practicing an inventive concept described herein. As used herein, the singular forms “a,” “an” and “the” are intended to include the plural forms as well, unless the context clearly indicates otherwise.

It will be further understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be understood that, although the terms first, second, third etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of one or more of these.

Spatially relative terms, such as “beneath,” “below,” “lower,” “above,” “upper” and the like may be used to describe an element and/or feature's relationship to another element(s) and/or feature(s) as, for example, illustrated in the figures. It will be understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g., rotated 90 degrees or at other orientations) and the spatially relative descriptors used herein interpreted accordingly.

The term “and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination. For example, it will be appreciated that all features set out in any of the claims (whether independent or dependent) can be combined in any given way.

As described herein, “room pressure” shall mean pressure of the environment surrounding the systems and devices of the present inventive concepts. Positive pressure includes pressure above room pressure or simply a pressure that is greater than another pressure, such as a positive differential pressure across a fluid pathway component such as a valve. Negative pressure includes pressure below room pressure or a pressure that is less than another pressure, such as a negative differential pressure across a fluid component pathway such as a valve. Negative pressure can include a vacuum but does not imply a pressure below a vacuum. As used herein, the term “vacuum” can be used to refer to a full or partial vacuum, or any negative pressure as described hereabove. As used herein, the term “vacuum level” refers to a measure of a vacuum wherein the lower the pressure, the greater the vacuum level.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross sectional area as the cross section of the component being described.

As used herein, the term “ablative temperature” refers to a temperature at which tissue necrosis or other desired tissue treatment occurs (e.g. a temperature sufficiently hot or sufficiently cold to cause tissue necrosis or any desired effect). As used herein, the term “ablative fluid” refers to one or more liquids, gases, gels or other fluids whose thermal properties cause tissue necrosis and/or another desired tissue treatment (e.g. one or more fluids at an ablative temperature). Alternatively or additionally, “ablative fluid” refers to one or more fluids whose chemical properties (at room temperature, body temperature or otherwise) cause tissue necrosis or another desired tissue treatment. A tissue treatment element (e.g. a functional element) of the present inventive concepts can comprise one or more ablative fluids and/or be configured to deliver one or more ablative fluids (e.g. deliver the fluids onto a tissue surface and/or into a volume of tissue).

As used herein, the term “threshold” refers to a maximum level, a minimum level, and/or range of values correlating to a desired or undesired state. In some embodiments, a system parameter is maintained above a minimum threshold, below a maximum threshold. within a threshold range of values and/or outside a threshold range of values, to cause a desired effect (e.g. efficacious therapy) and/or to prevent or otherwise reduce (hereinafter “prevent”) an undesired event (e.g. a device and/or clinical adverse event). In some embodiments, a system parameter is maintained above a first threshold (e.g. above a first temperature threshold to cause a desired therapeutic effect to tissue) and below a second threshold (e.g. below a second temperature threshold to prevent undesired tissue damage). In some embodiments, a threshold value is determined to include a safety margin, such as to account for patient variability, system variability, tolerances, and the like. As used herein, “exceeding a threshold” relates to a parameter going above a maximum threshold, below a minimum threshold, within a range of threshold values and/or outside of a range of threshold values.

As used herein, the term “proximate”, when used to describe proximity of a first component or location to a second component or location, is to be taken to include one or more locations near to the second component or location, as well as locations in, on and/or within the second component or location. For example, a component positioned proximate an anatomical site (e.g. a target tissue location), shall include components positioned near to the anatomical site, as well as components positioned in, on and/or within the anatomical site.

As used herein, the term “functional element” is to be taken to include one or more elements constructed and arranged to perform a function. A functional element can comprise a sensor and/or a transducer. In some embodiments, a functional element is configured to deliver energy and/or otherwise treat tissue (e.g. a functional element configured as a treatment element). Alternatively or additionally, a functional element (e.g. a functional element comprising a sensor) can be configured to record one or more parameters, such as a patient physiologic parameter; a patient anatomical parameter (e.g. a tissue geometry parameter); a patient environment parameter; and/or a system parameter. In some embodiments, a sensor or other functional element is configured to perform a diagnostic function. In some embodiments, a functional element comprises one or more elements constructed and arranged to perform a function selected from the group consisting of: deliver energy; extract energy (e.g. to cool a component); deliver a drug or other agent; manipulate a system component or patient tissue; record or otherwise sense a parameter such as a patient physiologic parameter or a patient anatomical parameter; and combinations of one or more of these. A functional element can comprise a fluid, such as an ablative fluid (as described hereabove) comprising a liquid or gas configured to ablate or otherwise treat tissue. A functional element can comprise a reservoir, such as an expandable balloon configured to receive an ablative fluid. A “functional assembly” can comprise an assembly constructed and arranged to perform a function, such as is described hereabove. In some embodiments, a functional assembly is configured to deliver energy and/or otherwise treat tissue (e.g. a functional assembly configured as a treatment assembly). Alternatively or additionally, a functional assembly can be configured to record one or more parameters, such as a patient physiologic parameter; a patient anatomical parameter; a patient environment parameter; and/or a system parameter. A functional assembly can comprise an expandable assembly. A functional assembly can comprise one or more functional elements.

As used herein, the term “transducer” is to be taken to include any component or combination of components that receives energy or any input, and produces an output. For example, a transducer can include an electrode that receives electrical energy, and distributes the electrical energy to tissue (e.g. based on the size of the electrode). In some configurations, a transducer converts an electrical signal into any output, such as light (e.g. a transducer comprising a light emitting diode or light bulb), sound (e.g. a transducer comprising a piezo crystal configured to deliver ultrasound energy), pressure, heat energy, cryogenic energy, chemical energy, mechanical energy (e.g. a transducer comprising a motor or a solenoid), magnetic energy, and/or a different electrical signal. Alternatively or additionally, a transducer can convert a physical quantity (e.g. variations in a physical quantity) into an electrical signal. A transducer can include any component that delivers energy and/or an agent to tissue, such as a transducer configured to deliver one or more of: heat energy to tissue; cryogenic energy to tissue; electrical energy to tissue (e.g. a transducer comprising one or more electrodes); light energy to tissue (e.g. a transducer comprising a laser, light emitting diode and/or optical component such as a lens or prism); mechanical energy to tissue (e.g. a transducer comprising a tissue manipulating element); sound energy to tissue (e.g. a transducer comprising a piezo crystal); chemical energy; electromagnetic energy; magnetic energy; and combinations of one or more of these. Alternatively or additionally, a transducer can comprise a mechanism, such as a valve, a grasping element, an anchoring mechanism, an electrically-activated mechanism, a mechanically-activated mechanism and/or a thermally activated mechanism.

As used herein, the term “tissue contacting surface” refers to a surface of a system or device component that makes physical contact with tissue, such as a portion of an external surface of an expandable component (e.g. a portion of a balloon's surface) which contacts tissue once expanded. In some embodiments, tissue contacting a tissue contacting surface directly receives energy from the tissue contacting surface of the expandable components, however tissue in proximity (e.g. below or alongside) also receives energy (e.g. via conduction of the delivered energy and/or a resultant energy).

As used herein, the term “conduit” refers to one or more lumens, spaces (e.g. space between two concentric shafts), tubes and/or other conduits including at least one lumen and/or other passageways through which fluid can be transported from one location to another. Conduit can also refer to an outer conduit that surrounds one or more inner conduits, the one or more inner conduits transporting fluid from one location to another. For example, an outer conduit can function as a trajectory-determining guide tube that provides a desired trajectory for the one or more inner, fluid-carrying conduits. A conduit can refer to one or more lumens, spaces (e.g. space between two concentric shafts), tubes and/or other conduits including at least one lumen, and/or other passageways through which a wire (e.g. a guidewire or an electrical conductor), a rod, an optical fiber, a linkage, one or more inner conduits, and/or another filament can be positioned and/or translated within.

As used herein, the term “fluid” can refer to a liquid, gas, gel, or any flowable material, such as a material which can be propelled through a conduit of the present inventive concepts.

It is an object of the present inventive concepts to provide systems, methods and devices for safely and effectively treating and/or diagnosing a volume of tissue (the “target tissue”), such as to treat and/or diagnose a patient disease or disorder. As used herein, the term “treat” shall include “diagnose”, and vice versa. Target tissue can comprise one or more target tissue segments or other target tissue portions, such as target tissue located in the intestine of a patient. Clinical procedures in the duodenum and other locations of the small intestine are challenging for a number of reasons, such as those caused by the long distance between the mouth and the intestine and the complexities of the gastrointestinal passageway encountered (including passage through the stomach) during device (e.g. catheter) insertion and operation.

Intestinal diameter varies along its length, and effective devices must accommodate this variation. The intestine is quite distensible in the longitudinal and radial directions, further complicating device (e.g. catheter) manipulation and operation (e.g. delivery of energy to tissue). Mobility of intestinal mucosa relative to muscularis is present, as well as mobility of the full wall, but can result in undesired stretching, compression and intussusception. The duodenum is normally relatively closed, and requires insufflation to open (e.g. for visualization and/or manipulation of inserted devices). The insufflation medium (e.g. gas) moves through the intestine, so additional gas can be delivered if required. Duodenal and other intestinal tissue tends to stretch or compress as a device is advanced or retracted, respectively, such as to cause retrograde expulsion of devices if a stabilization force is not maintained. It is difficult to manipulate and control devices that include treatment, diagnostic, and/or other elements positioned in the small intestine. The small intestine wraps around the pancreas, and the curvature is quite variable from patient to patient.

The length of the intestine along an outer curve is longer than that along an inner curve. In many procedures, there is a desire to avoid damage to the ampulla of Vater (e.g. to avoid restricting bile and/or pancreatic fluid), tissue which can be difficult to visualize or otherwise identify. There are relatively few endoscopically visualizable landmarks in the intestine, making it difficult to know where in the intestine a portion (e.g. a distal portion) of a device is positioned. Access to the intestine through the stomach via an over-the wire catheter loses one-to-one motion between a proximal handle and a distal portion of the device, as slack can accumulate in the stomach during advancement and slack can be relieved from the stomach during withdrawal. Accessing the intestine can include entering the intestine through the pylorus, a small sphincter, from the stomach, and in obese patients, large stretchable stomachs make it difficult to direct a device to the pylorus. The intestinal mucosa has a very irregular surface due to plicae circulares and mucosal villi, and performing a treatment (e.g. an ablation treatment) of the intestinal mucosa is quite different from a treatment procedure performed in the stomach or esophagus, because of this irregularity. Peristalsis present in the small intestine is dynamic and unpredictable and can alter functional element, functional assembly and/or other device component position and/or contact level with tissue. The intestine is not only thin-walled, but the thickness of the wall is highly variable, even within small axial segments of the small intestine, thus complicating preferential ablation of inner layers versus outer layers of the small intestine. The muscularis is innervated and scars and/or stenoses easily, and as such, even minimal trauma to the muscularis should be avoided.

Target tissue can comprise one or more layers of a portion of tubular or non-tubular tissue, such as tissue of an organ or tissue of the gastrointestinal (GI) tract of a patient, such as tissue of the small intestine or large intestine. The systems and devices of the present inventive concepts can include one or more functional assemblies and/or functional elements configured to treat target tissue, such as a treatment element comprising fluid at an ablative temperature delivered to a balloon (ablative temperature fluid and/or balloon filled with ablative fluid each referred to singly or collectively as a “functional element” or a “treatment element” of the present inventive concepts). One or more functional elements can be provided in, on and/or within an expandable functional assembly or other radially deployable mechanism. Functional assemblies and/or functional elements can be configured to treat target tissue (e.g. deliver energy to target tissue), such as to modify target tissue (e.g. to modify the secretions from the target tissue and/or absorption of the target tissue), ablate target tissue (e.g. to cause the replacement of the target tissue with “new tissue”) and/or to cause a reduction in the surface area of target tissue (e.g. the luminal surface area of an inner wall of tubular tissue) at and/or proximate to one or more locations where the treatment was performed (e.g. at and/or proximate the location where energy was delivered). The luminal surface treatment, and/or other tissue treatment, can occur acutely and/or it can take place over time, such as days, weeks or months. A tissue surface area reduction can correspond to a reduction in mucosal surface area available to function in an absorptive, neuronal signaling, and/or a hormonal secretory capacity. A target tissue treatment can result in the replacement of target tissue with new tissue with different absorptive and/or secretory capacity and/or other desirable effect related to replacement and/or modification of target tissue. The treatment of target tissue with the systems, devices and methods of the present inventive concepts can provide a therapeutic benefit to the patient, such as to treat one or more diseases or disorders of the patient, as described in detail herebelow.

Each functional assembly (e.g. treatment assembly and/or diagnostic assembly) can comprise at least one functional element (e.g. tissue treatment element and/or tissue diagnostic element) such as a functional element selected from the group consisting of: ablative fluid delivered to a balloon or other expandable fluid reservoir; energy delivery element mounted to an expandable functional assembly such as an electrode or other energy delivery element configured to deliver radiofrequency (RF) energy and/or microwave energy; light delivery element configured to deliver laser or other light energy; fluid delivery element (e.g. needle or nozzle) configured to deliver ablative fluid directly onto and/or into tissue; sound delivery element such as an ultrasonic and/or subsonic sound delivery element; and combinations of one or more of these. Numerous forms of functional assemblies and/or functional elements can be included. In some embodiments, the functional assemblies and/or the one or more functional elements contained therein are configured as described in: applicant's co-pending U.S. patent application Ser. No. 13/945,138, entitled “Devices and Methods for the Treatment of Tissue”, filed Jul. 18, 2013; applicant's co-pending U.S. patent application Ser. No. 14/470,503, entitled “Heat Ablation Systems, Devices and Methods for the Treatment of Tissue”, filed Aug. 27, 2014; applicant's co-pending U.S. patent application Ser. No. 14/609,332, entitled “Electrical Energy Ablation Systems, Devices and Methods for the Treatment of Tissue”, filed Jan. 29, 2015; and/or applicant's co-pending U.S. patent application Ser. No. 14/609,334, entitled “Ablation Systems, Devices and Methods for the Treatment of Tissue”, filed Jan. 29, 2015; the content of each of which is incorporated herein by reference in its entirety for all purposes.

The treatment assemblies and/or treatment elements of the present inventive concepts can be constructed and arranged to deliver one or more treatments (e.g. deliver energy, deliver a chemically ablative fluid, mechanically abrade and/or otherwise treat tissue) directly to a particular area of tissue, the “delivery zone”. During a single delivery of treatment, a treatment element can be constructed and arranged to deliver treatment to a relatively continuous surface of tissue (e.g. a continuous surface of tissue in contact with a balloon filled with ablative fluid or a surface of tissue onto which a chemically ablative fluid is sprayed, coated or otherwise delivered). In these continuous-surface treatment delivery embodiments, the delivery zone comprises the continuous surface of tissue receiving the treatment directly. Alternatively, a treatment element can be constructed and arranged to deliver treatment to multiple discrete portions of a tissue surface, with one or more tissue surface portions in-between other surface portions that do not directly receive energy or other treatment from the treatment element. In these segmented-surface treatment delivery embodiments, the delivery zone is defined by a periphery of the multiple tissue surface area portions receiving treatment, similar to a “convex hull” or “convex envelope” used in mathematics to define an area including a number of discrete locations that define a periphery. A delivery zone can comprise two or more contiguous or non-contiguous delivery zones, and multiple delivery zones can be treated sequentially and/or simultaneously.

For example, in embodiments where the treatment element is hot fluid (e.g. ablative fluid at a sufficiently high temperature to cause tissue necrosis) positioned within a balloon, the delivery zone comprises all tissue surfaces contacted by the balloon that directly receive ablative thermal energy from the ablative fluid through the balloon. In embodiments where the treatment element is a balloon filled with cold fluid (e.g. ablative fluid at a sufficiently low temperature to cause tissue necrosis), the delivery zone can comprise all tissue surfaces contacted by the balloon that have heat directly extracted from them by the cold fluid (e.g. at a sufficient cold temperature to treat the tissue). In embodiments where the treatment element is an array of electrodes configured to deliver electrical energy (e.g. RF energy) to tissue, the delivery zone can comprise an area defined by the electrodes on the periphery of the array (e.g. a convex hull as described above), such as when the electrodes are positioned and energy is delivered to treat relatively the entire surface of tissue within the periphery. In embodiments where the treatment element comprises one or more fluid delivery elements delivering ablative fluid directly onto tissue (e.g. an ablative fluid whose chemical nature modifies tissue, at body temperature or otherwise), the delivery zone can comprise a surface defined by the periphery of tissue locations receiving the ablative fluid, such as when the ablative fluid is delivered (e.g. sprayed or otherwise applied, such as via a sponge) to relatively the entire surface within the periphery. In embodiments where the treatment element comprises one or more light delivery elements such as those that deliver laser energy to tissue, the delivery zone can comprise a surface area defined by the periphery of tissue locations receiving the light energy, such as when light is delivered at a set of locations and with a magnitude of energy configured to treat relatively the entire surface of tissue within the periphery. In these embodiments, light can be delivered to relatively the entire energy delivery zone, or to a large number (e.g. greater than 100) of tissue locations within the periphery of the delivery zone (e.g. making up less than 50%, less than 20% or less than 10% of the total surface area of the delivery zone). In embodiments where the treatment element comprises one or more sound delivery elements such as those that deliver sub-sonic and/or ultrasonic sound energy to tissue, the delivery zone can comprise a surface area defined by the periphery of tissue locations receiving the sound energy, such as when ablative sound energy is delivered at a set of locations and with a magnitude of energy configured to treat relatively the entire surface of tissue within the periphery. In embodiments in which the treatment element comprises a mechanical cutter or other abrasion element, the delivery zone can comprise a surface defined by all tissue dissected, cut, mechanically disrupted and/or otherwise modified during a single abrading step of the mechanical abrader.

A delivery zone can comprise a cumulative set of delivery zones that receive treatment simultaneously and/or sequentially, by one or more tissue treatment elements, such as those described herein. A delivery zone can comprise a first delivery zone defined when a treatment element treats target tissue in a first treatment delivery, plus a second delivery zone defined when the treatment element treats target tissue in a second treatment delivery, and so on. In these embodiments, the treatment element can be translated, rotated and/or otherwise repositioned between treatments (e.g. energy delivery), where each delivery zone is associated with the position of the treatment element during each treatment. Multiple delivery zones can receive treatment in a single procedure, such as within a period of less than twenty-four hours. A delivery zone can comprise a set of multiple delivery zones treated by two or more treatment elements.

Target tissue treated by each energy delivery and/or other treatment delivery comprises the tissue directly receiving treatment (i.e. the tissue defined by the delivery zone) plus “neighboring tissue” which is also modified by the associated treatment delivery. The neighboring tissue can comprise tissue alongside, below (e.g. in a deeper tissue layer) and/or otherwise proximate the delivery zone tissue. The neighboring tissue treatment can be due to one or more of: conduction and/or convection of heat or cold from the delivery zone; flow of ablative fluid from the delivery zone; flow of toxins or other agents that occur during cell degradation and/or cell death; radiation; luminescence, light dissipation; and other energy and/or chemical propagation mechanisms. In some embodiments, an area (i.e. the delivery zone) comprising an inner surface of mucosal tissue directly receives treatment from one or more treatment elements (e.g. an ablative fluid contained within a balloon), and the total volume of target tissue treated by that single treatment delivery includes: the delivery zone tissue (i.e. surface mucosal tissue directly receiving energy and/or other treatment from the treatment element); surface mucosal tissue in close proximity (e.g. adjacent) to the delivery zone tissue; and mucosal and potentially submucosal tissue layers beneath (deeper than) the delivery zone tissue and the treated adjacent surface mucosal tissue.

In some embodiments, a “treatment neutralizing” procedure is performed after one or more treatments (e.g. energy deliveries), such as a treatment neutralizing cooling procedure performed after one or more treatment elements deliver heat to treat target tissue, or a treatment neutralizing warming procedure performed after one or more treatment elements deliver cryogenic energy to treat target tissue. In these embodiments, the treatment neutralizing cooling or warming fluid can be delivered to the same functional assembly (e.g. an expandable functional assembly comprising a balloon) delivering the heat or cryogenic treatment, respectively, and/or the neutralizing fluid can be delivered directly to tissue by the same or different functional assembly or functional element. In some embodiments, a functional element delivers an ablating agent to target tissue (e.g. a chemical or other agent configured to cause target tissue necrosis or otherwise treat target tissue), and a treatment neutralizing procedure comprises delivery of a neutralizing agent (by the same or different functional element) to target and/or non-target tissue to reduce continued ablation due to the delivered caustic ablative fluid (e.g. a base to neutralize a delivered acid or an acid to neutralize a delivered base).

The treatment assemblies and/or other functional assemblies of the present inventive concepts can include one or more functional elements configured as fluid delivery elements. The one or more functional elements can comprise one or more needles, nozzles and/or fluid jets configured to deliver one or more fluids or other injectates to tissue, such as to expand target tissue and/or tissue proximate the target tissue (e.g. safety margin tissue) prior to treatment of target tissue by a tissue treatment element. The expanded tissue layer acts as a safety volume of tissue, reducing the specificity of the treatment (e.g. ablation) required and/or the need to protect the underlying non-target tissue from damage. In some embodiments, a vacuum pressure can be used to manipulate tissue and/or to maintain proximity between a portion of a tissue expansion device and tissue. The vacuum can be provided by one or more vacuum sources, such as via one or more operator adjustable vacuum sources. The functional assemblies can include a tissue capture port configured to receive the vacuum and engage tissue (e.g. tissue to receive fluid via one or more fluid delivery elements).

Each functional assembly and/or functional element of the present inventive concepts can be configured to be positioned in one or more intestinal and/or other locations of the patient, such as to perform a function (e.g. perform a treatment, deliver fluid and/or record data) at one or more contiguous or discontiguous tissue locations. Target tissue to be treated (e.g. ablated) comprises a three-dimensional volume of tissue, and can include a first portion, a treatment portion, whose treatment has a therapeutic benefit to a patient; as well as a second portion, a “safety-margin” portion, whose treatment has minimal or no adverse effects to the patient. “Non-target tissue” can be identified (e.g. prior to and/or during the medical procedure), wherein the non-target tissue comprises tissue whose treatment by the treatment assembly and/or treatment element should be reduced or avoided such as to reduce or prevent an undesired effect to the patient.

The target tissue treatment can cause one or more modifications of the target tissue such as a modification selected from the group consisting of: modification of cellular function; cell death; apoptosis; instant cell death; cell necrosis; denaturing of cells; removal of cells; and combinations of one or more of these. In some embodiments, the target tissue treatment is configured to create scar tissue. Target tissue can be selected such that after treatment the treated target tissue and/or the tissue that replaces the target tissue functions differently than the pre-treated target tissue, such as to have a therapeutic benefit for the patient. The modified and/or replacement tissue (singly or collectively “treated tissue”) can exhibit different properties than the pre-treated target tissue, such as different properties that are used to treat a patient disease or disorder. The treated tissue can have different secretions and/or quantities of secretions than the pre-treated target tissue, such as to treat diabetes, hypercholesterolemia and/or another patient disease or disorder. The treated tissue can have different absorptive properties than the target tissue, such as to treat diabetes, hypercholesterolemia and/or another patient disease or disorder. The treated tissue can have a different surface topography than the target tissue, such as a modification of the topography of the inner wall of the GI tract that includes a smoothing or flattening of its inner surface, such as a modification in which the luminal surface area of one or more segments of the GI tract is reduced after treatment. The effect of the treatment (e.g. the effect on the target tissue) can occur acutely, such as within twenty-four hours, or after longer periods of time, such as greater than twenty-four hours or greater than one week.

Target tissue to be treated can comprise two or more discrete tissue segments, such as two or more axial segments of the GI tract. Each tissue segment can comprise a full (e.g. approximately 360°) or partial circumferential segment of the tissue segment. Multiple tissue segments can be treated with the same or different functional elements (e.g. treatment elements), and they can be treated simultaneously or in sequential steps (e.g. sequential energy delivery steps that deliver energy to multiple delivery zones). Multiple tissue segments can be treated in the same or different clinical procedures (e.g. procedures performed on different days). In some embodiments, a series of tissue segments comprising a series of axial segments of the GI tract are treated in a single clinical procedure. The first and second tissue segments can be directly adjacent, they can contain overlapping portions of tissue, and there can be gaps between the segments. Dissimilarities in treatment elements can include type and/or amount of energy to be delivered by an energy delivery based treatment element. Dissimilarities in target tissue treatments can include: target tissue area treated; target tissue volume treated; target tissue length treated; target tissue depth treated; target tissue circumferential portion treated; ablative fluid type, volume and/or temperature delivered to a reservoir such as a balloon; ablative fluid type, volume and/or temperature delivered directly to tissue; energy delivery type; energy delivery rate and/or amount; peak energy delivered; average temperature of target tissue achieved during target tissue treatment; maximum temperature achieved during target tissue treatment; temperature profile of target tissue treatment; duration of target tissue treatment; surface area reduction achieved by target tissue treatment; and combinations of one or more of these.

Target tissue can include tissue of the duodenum, such as tissue including substantially all or a portion of the mucosal layer of one or more axial segments of the duodenum (e.g. including all or a portion of the plicae circulares), such as to treat diabetes, hypercholesterolemia and/or another patient disease or disorder, such as while leaving the duodenum anatomically connected after treatment. Target tissue can include one or more portions of a tissue layer selected from the group consisting of: mucosa; mucosa through superficial submucosa; mucosa through mid-submucosa; mucosa through deep-submucosa; and combinations of one or more of these. Replacement tissue can comprise cells that have migrated from one or more of: gastric mucosa; jejunal mucosa; an untreated portion of the duodenum whose mucosal tissue functions differently than the treated mucosal tissue functions prior to treatment; and combinations of one or more of these. Replacement tissue can include one or more tissue types selected from the group consisting of: scar tissue; normal intestinal mucosa; gastric mucosa; and combinations of one or more of these. In some embodiments, replacement tissue comprises tissue that has been delivered onto and/or into tissue by a catheter of the present inventive concepts. In some embodiments, target tissue includes a treatment portion comprising the mucosal layer of the duodenum, and a safety-margin portion comprising a near-full or partial layer of the submucosal layer of the duodenum. In some embodiments, the target tissue comprises nearly the entire mucosal layer of the duodenum, and can include a portion of the pylorus contiguous with the duodenal mucosa and/or a portion of the jejunum contiguous with the duodenal mucosa. In some embodiments, the target tissue comprises all or a portion of the duodenal mucosa distal to the ampulla of Vater (e.g. avoiding tissue within at least 0.5 cm, 1.0 cm or 1.5 cm from the ampulla of Vater while including tissue within 5 cm, 10 cm or 15 cm distal to the ampulla of Vater). In these embodiments, the target tissue can comprise at least 10%, at least 15%, at least 25%, at least 30% or at least 50% of the duodenal mucosa distal to the ampulla of Vater. Alternatively or additionally, the target tissue can comprise no more than 70% or no more than 90% of the duodenal mucosa distal to the ampulla of Vater. In these embodiments, tissue proximal to and/or proximate the ampulla of Vater can comprise non-target tissue (i.e. tissue whose treatment is avoided or at least reduced).

In some embodiments, the target tissue comprises at least a portion of duodenal mucosal tissue, and the systems, methods and devices of the present inventive concepts are configured to counteract duodenal mucosal changes that cause an intestinal hormonal impairment leading to insulin resistance in patients. In these embodiments, the therapy provided can improve the body's ability to process sugar and dramatically improve glycemic control for patients with insulin resistance and/or Type 2 diabetes. In some embodiments, target tissue is treated to prevent and/or reduce cognitive decline (e.g. Alzheimer's Disease), such as by improving sugar metabolism in the brain, overcoming insulin resistance in the brain, reducing toxicity of beta amyloid, reducing oxidative stress, and/or reducing inflammation in the brain associated with neuronal death. In some embodiments, target tissue is treated to: prevent liver fibrosis and/or cirrhosis (e.g. non-alcoholic fatty liver disease NAFLD or non-alcoholic steatohepatitis NASH); reduce liver fat; reduce oxidative stress; and/or reduce inflammation in the liver associated with liver fibrosis and toxicity.

Hormones released from the intestinal mucosa play an important role in modulating glucose homeostasis, and different axial segments of the intestinal mucosa release different hormones in the fasting and post-prandial state, in order to modulate blood glucose in the fasting and post-prandial states, respectively. After a meal, the proximal intestinal mucosa senses the intestine for ingested glucose and releases a collection of hormones in response to this signal.

These hormones initiate the process of insulin release into the bloodstream after a meal, but they also induce some insulin resistance to prevent the released insulin from causing hypoglycemia before the body has a chance to absorb the ingested glucose. One such hormone that plays a role in this is GIP. Distal gut hormones (produced in the jejunum or a more distal location), on the contrary, allow the release of more insulin but also play a role in helping the body now become sensitive to its circulating insulin. Teleologically, the explanation for this difference in the type of gut hormones produced by different segments of the intestine is that enough glucose will have been absorbed by the time nutrients reach the distal intestine to allow the insulin to begin to function to reduce blood glucose levels. Releasing different hormones at different times (e.g. from different segments of the intestine) enables the body to absorb and process glucose in such a way as to avoid hypoglycemia (blood sugars that are too low) and hyperglycemia (blood sugars that are too high). In this way, intestinal hormonal signaling is important for whole body glucose homeostasis in the fasting and post-prandial states. The treatment can also lead to weight loss through decreased absorption of nutrients, increased sensation of satiety, altered food preferences, increased energy expenditure, and combinations of one or more of these.

In patients with Type 2 Diabetes, a lifetime of exposure to fat and sugar can lead to intestinal changes that occur in regions with the highest exposure to these nutrients, predominantly in the proximal intestine. These changes are characterized by an excess proximal intestinal mucosa's hormonal contribution to the fasting and post-prandial glucose homeostasis. The net result of these intestinal changes is to create a condition of insulin resistance and impaired glucose tolerance. Treatment of duodenal mucosal tissue with the systems, devices and methods of the present inventive concepts can be performed to alter the intestinal mucosal hormone production from the region of treated tissue. The treated tissue can then have an altered hormonal secretion pattern that affects blood glucose levels in the fasting and post-prandial states. The tissue treatment of the present inventive concepts can be performed to effect duodenal mucosal tissue secretion of GIP and/or GLP-1. The tissue treatment can lead to changes in the blood levels of GIP and/or GLP-1 (and other gut hormones) that can lead to changes in glucose homeostasis in the fasting and/or post-prandial states. The treatment can lead to changes in insulin and/or glucagon secretion from the pancreas and/or insulin and/or glucagon levels in the bloodstream. The treatment can lead to changes in pancreatic beta cell function and/or health through direct hormonal consequences of the treated duodenal tissue and/or indirectly through improved blood glucose levels. In some embodiments, the treatment of the present inventive concepts is configured to at least one of reduce a blood glucose level and/or reduce a lipoprotein level.

Treatment of intestinal tissue (e.g. duodenal mucosal tissue) can be performed to treat a disease and/or disorder selected from the group consisting of: diabetes; pre-diabetes; impaired glucose tolerance; insulin resistance; obesity or otherwise being overweight; a metabolic disorder and/or disease; and combinations of one or more of these. In some embodiments, treatment of intestinal tissue (e.g. at least duodenal mucosal tissue) using the systems, devices and/or methods of the present inventive concepts can be performed to treat one or more disease and/or disorder selected from the group consisting of: Type 2 diabetes; Type 1 diabetes; “Double diabetes”; gestational diabetes; hyperglycemia; pre-diabetes; impaired glucose tolerance; insulin resistance; non-alcoholic fatty liver disease (NAFLD); non-alcoholic steatohepatitis (NASH); obesity; obesity-related disorder; polycystic ovarian syndrome (PCOS); hypertriglyceridemia; hypercholesterolemia; psoriasis; GERD; coronary artery disease (e.g. as a secondary prevention); stroke; TIA; cognitive decline; dementia; Alzheimer's; neuropathy; diabetic nephropathy; retinopathy; heart disease; diabetic heart disease; heart failure; diabetic heart failure; hirsutism; hyperandrogenism; fertility issues; menstrual dysfunction; cancer such as liver cancer, ovarian cancer, breast cancer, endometrial cancer, cholangiocarcinoma, adenocarcinoma, glandular tissue tumor(s), stomach cancer, large bowel cancer, and/or prostate cancer; diastolic dysfunction; hypertension; myocardial infarction; microvascular disease related to diabetes; Alzheimer's Disease; sleep apnea; and combinations of one or more of these. A near full circumferential portion (e.g. approximately 360°) of the mucosal layer of one or more axial segments of GI tissue can be treated. In some embodiments, less than 360° of one or more axial segments of tubular tissue is treated, such as one or more circumferential portions less than 350°, or between 300° and 350°, such as to prevent a full circumferential scar from being created at the one or more axial segment locations.

Target tissue can be selected to treat two or more patient diseases or disorders, such as two or more patient diseases or disorders as described herein.

Target tissue can comprise tissue of the terminal ileum, such as to treat hypercholesterolemia and/or diabetes. In these embodiments, the target tissue can extend into the proximal ileum and/or the colon.

Target tissue can comprise gastric mucosal tissue, such as tissue regions that produce ghrelin and/or other appetite regulating hormones, such as to treat obesity and/or an appetite disorder.

Target tissue can comprise tissue selected from the group consisting of: large and/or flat colonic polyps; margin tissue remaining after a polypectomy; and combinations of one or more of these. These tissue locations can be treated to treat residual cancer cells.

Target tissue can comprise at least a portion of the intestinal tract afflicted with inflammatory bowel disease, such that Crohn's disease and/or ulcerative colitis can be treated.

Target tissue can comprise GI tissue selected to treat Celiac disease and/or to improve intestinal barrier function.

The functional assemblies, functional elements, systems, devices and methods of the present inventive concepts can be configured to avoid ablating or otherwise adversely affecting certain tissue, termed “non-target tissue” herein. Depending on the location of tissue intended for treatment (i.e. target tissue), different non-target tissue can be applicable. In certain embodiments, non-target tissue can comprise tissue selected from the group consisting of: gastrointestinal adventitia; duodenal adventitia; the tunica serosa; the tunica muscularis; the outermost partial layer of the submucosa; ampulla of Vater (also known as the papilla); pancreas; bile duct; pylorus; and combinations of one or more of these.

In some embodiments, two or more clinical procedures are performed in which one or more volumes of target tissue are treated in each clinical procedure, such as is described in applicant's co-pending U.S. patent application Ser. No. 14/673,565 , entitled “Methods, Systems and Devices for Performing Multiple Treatments on a Patient”, filed Mar. 30, 2015. For example, a second clinical procedure can be performed at least twenty-four hours after the first clinical procedure, such as a second clinical procedure performed within 6 months of a first clinical procedure or a clinical procedure performed after at least 6 months after the first clinical procedure. The first and second clinical procedures can be performed using similar or dissimilar methods, and they can be performed using similar or dissimilar systems and/or devices (e.g. performed with similar or dissimilar treatment and/or other functional elements). The first and second clinical procedures can treat similar or dissimilar volumes of target tissue (e.g. similar or dissimilar amounts of tissue treated and/or locations of tissue treated), and they can deliver energy to similar or dissimilar sets of multiple delivery zones. In some embodiments, the first and second clinical procedures can include treating and/or delivering energy to contiguous and/or overlapping regions of the GI tract either in the circumferential and/or axial dimensions. In other embodiments, the first and second clinical procedures can include the treatment of disparate regions of the GI tract (such as disparate regions of the duodenum, ileum, and/or stomach). The first and second clinical procedures can be performed using similar or dissimilar devices (e.g. catheters). The first and second clinical procedures can comprise similar or dissimilar deliveries of energy to treat the target tissue. The first and second clinical procedures can be performed at similar or dissimilar temperatures. The second clinical procedure can be performed based on diagnostic results collected after the first clinical procedure has been performed, such as when the diagnostic results are based on a biopsy of mucosal tissue.

The functional assemblies, treatment assemblies, treatment elements and other functional elements of the present inventive concepts can comprise an expandable element or otherwise be configured to automatically and/or manually expand or traverse in at least one radial direction. Typical expandable elements include but are not limited to: an inflatable balloon; a radially expandable cage or stent; one or more radially deployable arms; an expandable helix; an unfurlable compacted coiled structure; an unfurlable sheet; an unfoldable compacted structure; and combinations of one or more of these. In some embodiments, an expandable element can comprise a radially expandable tube, such as a sheet of material resiliently biased in a radially expanded condition that can be compacted through a furling operation, or a sheet of material resiliently biased in a radially compact condition that can be expanded through an unfurling operation. An expandable element can comprise a foldable sheet, such as a sheet configured to be folded to be radially compacted and/or to be unfolded to radially expand. In some embodiments, an expandable element expands to contact tissue, such as to expand to a diameter similar to the diameter of the luminal wall tissue into which the expandable element has been placed. In some embodiments, an expandable element expands to be closer to wall tissue, but remain at a distance (e.g. a fixed or pre-determined distance) from the tissue surface, such as when the tissue is subsequently brought into contact with all or a portion of an expanded functional assembly or functional element (e.g. using insufflation fluid withdrawal techniques). In some embodiments, an expandable element expands to be larger than the diameter of the luminal wall tissue into which the expandable element has been placed, such as to improve the quality of the apposition of the expandable element against the uneven surface of the tissue. In these embodiments, the fully expanded diameter of an expandable element would be configured to avoid a diameter large enough to cause lasting mechanical damage to the apposed tissue and/or to tissue proximate the apposed tissue. In some embodiments, the expansion of an expandable element (e.g. the expansion of an expandable functional assembly) is monitored and/or varied (e.g. decreased and/or increased), such as to accommodate or otherwise compensate for peristalsis or other muscle contractions that occur in the GI tract (e.g. contractions that occur when a foreign body is present in the GI tract) and/or varied to accommodate changes in GI lumen diameter imposed by aspects of the procedure itself.

Any device (e.g. catheter) of the present inventive concepts can include one or more functional elements comprising one or more treatment elements configured to deliver energy to one or more delivery zones, to treat at least a portion of target tissue. Any device can include one or more functional elements comprising one or more fluid delivery elements, such as one or more nozzles or needles configured to deliver fluid toward and/or into tissue. The fluid delivery elements can be constructed and arranged to deliver fluid to perform a function selected from the group consisting of: expanding one or more tissue layers; warming or cooling tissue; removing debris or other substance from a tissue surface; delivering energy to a delivery zone comprising a continuous or segmented surface; treating target tissue; and combinations of one or more of these. Any of the expandable functional assemblies of the present inventive concepts can include one or more other functional elements, such as are described herein. The treatment elements and/or other functional elements (e.g. fluid delivery elements) can be mounted on, within (e.g. within the wall) and/or inside of an expandable element such as a balloon or expandable cage. In some embodiments, one or more functional elements is not mounted to an expandable element, such as those attached to a shaft or other non-expandable catheter component.

In some embodiments, a catheter comprises at least one functional element configured to deliver energy to a delivery zone such as to ablate target tissue. Examples of ablation-based functional elements include but are not limited to: ablative fluids, such as hot or cold ablative fluids delivered to a balloon and/or directly to target tissue; one or more fluid delivery elements configured to deliver ablative fluid directly to target tissue; an RF and/or microwave energy delivery element such as one or more electrodes; an ultrasonic and/or subsonic transducer such as one or more piezo crystals configured to ablate tissue with ultrasonic or subsonic energy, respectively, sound waves; a laser energy delivery element such as one or more optical fibers, laser diodes, prisms and/or lenses; a rotating ablation element; a circumferential array of ablation elements; and combinations of one or more of these.

The expandable elements comprising balloons of the present inventive concepts can be divided into two general categories: those that are composed of a substantially elastic material, such as silicone, latex, low-durometer polyurethane, and the like; and those that are composed of a substantially inelastic material, such as polyethylene terephthalate (PET), nylon, high-durometer polyurethane and the like. A third category includes balloons which include both elastic and inelastic portions. Within the category of elastic balloons, two subcategories exist: a first sub-category wherein a combination of material properties and/or wall thickness can be combined to produce a balloon that exhibits a measurable pressure-threshold for inflation (i.e. the balloon becomes inflated only after a minimum fluidic pressure is applied to the interior of the balloon); and a second sub-category, wherein the balloon expands elastically until an elastic limit is reached which effectively restricts the balloon diameter to a maximum value. The individual properties of the balloons in each of these categories can be applied to one or more advantages in the specific embodiments disclosed herein, these properties integrated singly or in combination. By way of example only, one or more of the following configurations can be employed: a highly elastic balloon can be used to achieve a wide range of operating diameters during treatment (e.g. during operation a desired balloon diameter can be achieved by adjustment of a combination of fluid temperature and pressure); a substantially inelastic balloon or a balloon that reaches its elastic limit within a diameter approximating a target tissue diameter (e.g. a duodenal mucosal diameter) can be used to achieve a relatively constant operating diameter that will be substantially independent of operating pressure and temperature; a balloon with a pressure-threshold for inflation can be used to maintain an uninflated diameter during relatively low pressure conditions of fluid flow and then achieve a larger operating diameter at higher pressure conditions of flow. Pressure-thresholded balloons can be configured in numerous ways. In one embodiment, a balloon is configured to have a relatively thick wall in its uninflated state, such as to maximize an electrically and/or thermally insulating effect while the balloon is maintained in this uninflated state. The balloon can be further configured such that its wall thickness decreases during radial expansion (e.g. to decrease an electrically and/or thermally insulating effect). In another embodiment, a balloon is configured to have a relatively small diameter in its uninflated state (e.g. a diameter that is small relative to the inner diameter of tubular target tissue such as the diameter of the mucosal layer of duodenal wall tissue), such as to minimize or completely eliminate apposition between the balloon and the surrounding tissue to minimize heat, RF and/or other energy transfer into the surrounding tissue until the balloon is fully inflated. In another embodiment, a balloon and an ablation system or catheter are configured to circulate a flow of fluid through the balloon (e.g. an elastic balloon or an inelastic balloon) at a sufficiently low enough pressure to prevent apposition of the balloon or other catheter component with target tissue, such as to pre-heat one or more surfaces of the ablation system or ablation device that are in fluid communication with the balloon. In this configuration, when the balloon or other ablation element is positioned to deliver energy to target tissue, the temperature of the balloon or other ablation element will be at a desired level or it will rapidly and efficiently reach the desired level for treatment (i.e. minimal heat loss to the fluid path components due to the pre-heating or pre-cooling). These configurations provide a method of delivering energy to tissue with an ablative fluid filled balloon. A “thermal priming” procedure can be performed prior to one or more target tissue treatments, such as to improve thermal response time of one or more portions of the catheter. Ablative fluid filled balloon catheters as well as thermal priming devices and methods can be configured as is described in applicant's co-pending U.S. patent application Ser. No. 14/470,503, entitled “Heat Ablation Systems, Devices and Methods for the Treatment of Tissue”, filed Aug. 27, 2014, the content of which is incorporated herein by reference in its entirety for all purposes.

A fluid evacuation procedure can be performed on one or more internal locations of the catheters, functional assemblies and/or functional elements of the present inventive concepts, such as when a negative pressure is applied to purge or otherwise evacuate fluid from one or more locations. A fluid evacuation procedure can be performed prior to a thermal priming procedure and/or prior to delivering ablative fluid to a treatment element.

At times during target tissue treatment when it is desirable to initiate, increase and/or otherwise modify the treatment of tissue by one or more treatment elements (e.g. a fluid delivery element delivering ablative fluid, a mechanically abrasive element, a hot or cold fluid balloon delivering a thermal energy to tissue and/or an electrode delivering RF energy), the diameter of the treatment assembly and/or treatment element (e.g. the diameter of a balloon, deployable cage, expandable tube or other expandable assembly) can be increased in situ to move a treatment element closer to target tissue and/or to change the contact force between the treatment element and the target tissue. At times during treatment when it is desirable to stop or otherwise decrease the amount of tissue treatment, the diameter of the treatment assembly and/or treatment element can be reduced in situ, such as to prevent or otherwise reduce delivery of energy or other treatment to the target tissue by eliminating or reducing tissue contact of one or more treatment elements (e.g. electrodes, abrasive surfaces or ablative fluid-filled balloons). For those cases where the native diameter of the target tissue varies substantially within a delivery zone, then a highly elastic or compliant balloon or other expandable element can be employed, such as a balloon or deployable cage which can be adjusted to achieve a wide range of operating diameters.

Alternatively or additionally, to initiate, increase and/or otherwise modify the treatment of tissue by one or more functional elements (e.g. a fluid delivery element delivering ablative fluid, a mechanically abrasive element, a hot or cold fluid balloon delivering thermal energy to or from tissue and/or an electrode delivering RF energy), the diameter of the target tissue can be decreased in situ to move target tissue closer to a treatment element and/or to change the contact force between the target tissue and the treatment element. To stop or otherwise decrease ablation of tissue, the diameter of tissue neighboring a treatment element can be increased in situ, such as to prevent or otherwise reduce delivery of energy or other treatment to the target tissue by eliminating or reducing tissue contact of one or more treatment elements (e.g. electrodes, abrasive surfaces or ablative fluid filled balloons). The diameter of the tissue proximate a functional assembly can be increased or decreased, independent of the functional assembly diameter, by means of delivering and/or withdrawing a fluid, to and/or from a body lumen (e.g. a lumen of a segment of the intestine) surrounded by target tissue, such as by using standard GI insufflation techniques. Typical insufflation fluids include but are not limited to: gases such as carbon dioxide or air; liquids such as water or saline solution; and combinations of one or more of these. The insufflation fluids can be introduced through a catheter, through an endoscope such as an endoscope through which the catheter is inserted, and/or via another device placed proximate the target tissue. Delivery of insufflation fluids can be performed to move target tissue away from one or more functional elements, such as to stop transfer of energy to target tissue at the end of a treatment of target tissue as described hereinabove. Alternatively or additionally, delivery of insufflation fluids can be performed to manipulate tissue, such as to distend and/or elongate tissue. Extraction of these insufflation fluids and/or the application of a vacuum or other negative pressure can be used to decrease the diameter of the target tissue, such as to bring the target tissue in closer proximity to one or more functional elements and/or to increase the contact force between target tissue and one or more functional elements, also as described hereinabove. In this tissue diameter controlled approach, a functional assembly including a balloon that can be maintained at a substantially constant diameter can be desirable, such as a substantially inelastic balloon such as a balloon with an elastic-limit.

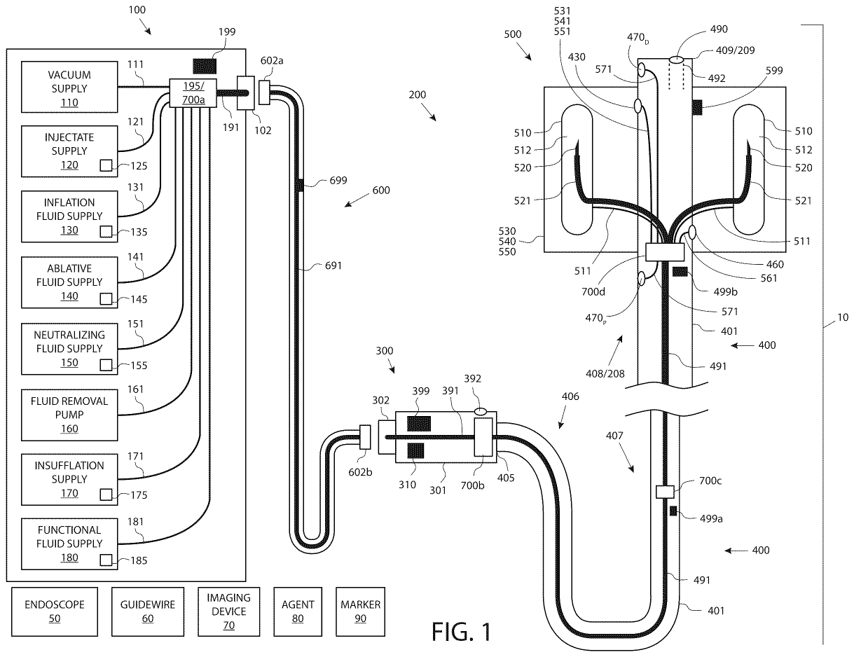

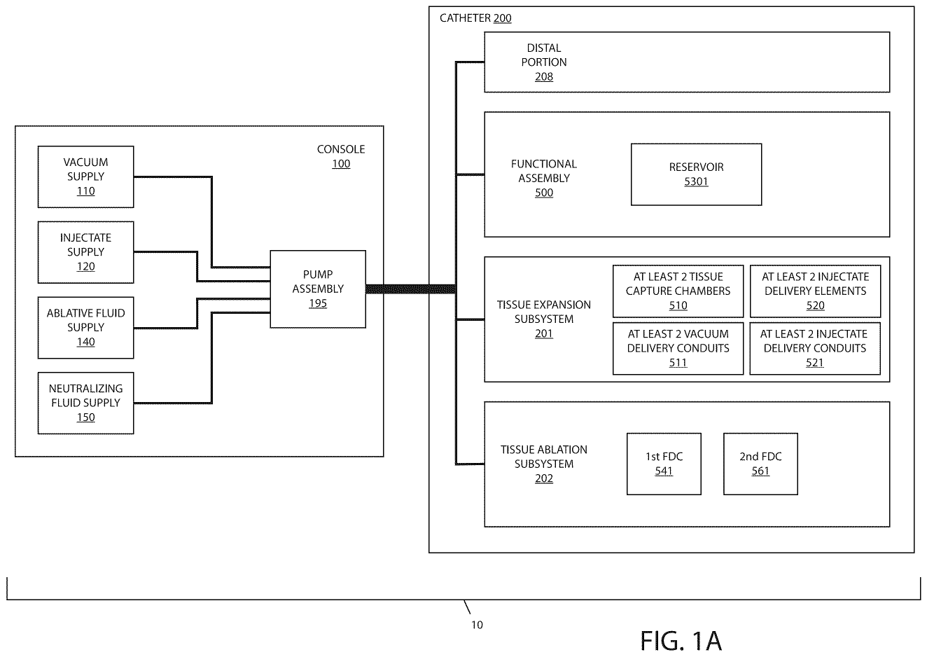

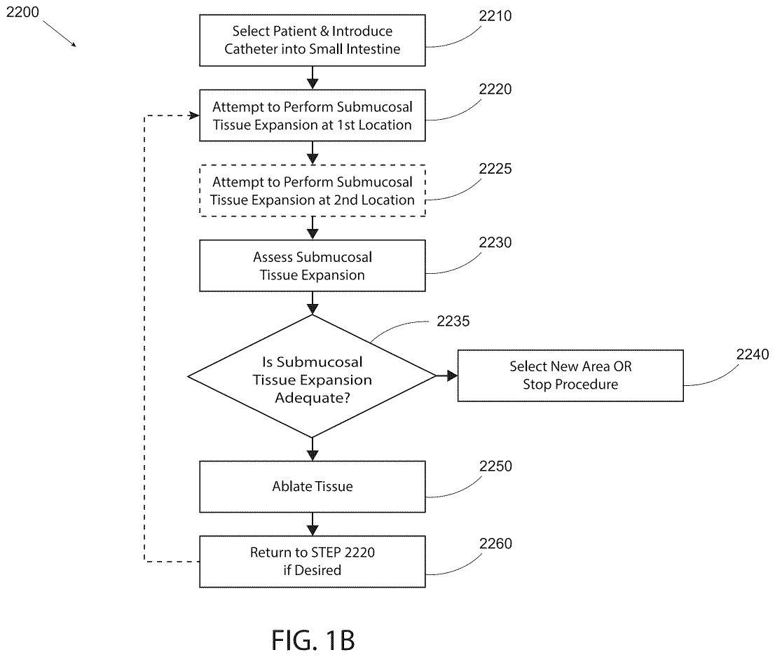

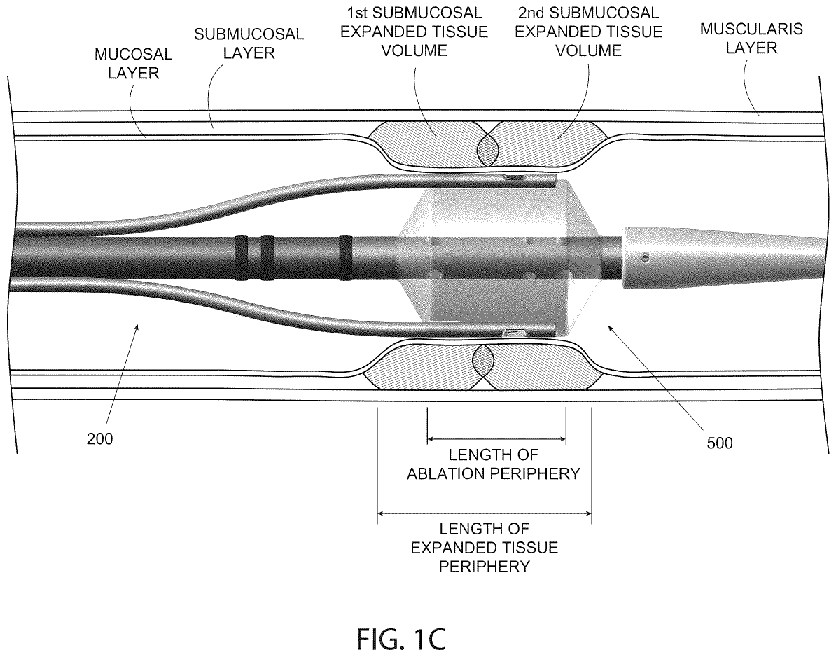

Referring now to FIG. 1, a system for treating and/or diagnosing (“treating” herein) gastrointestinal tissue is illustrated, consistent with the present inventive concepts. System 10 includes console 100 that operably attaches to a catheter, catheter 200. System 10 and catheter 200 can be used by an operator (e.g. one or more clinicians) to perform a therapeutic procedure and/or a diagnostic procedure. Catheter 200 can be constructed and arranged to treat and/or diagnose target tissue, such as tissue of the small intestine (e.g. mucosal tissue of the duodenum and/or jejunum) and/or other locations within the gastrointestinal (GI) tract. Catheter 200 can be constructed and arranged to ablate or remove tissue, such as by delivering energy to tissue.

Alternatively or additionally, catheter 200 can be constructed and arranged to expand one or more layers of tissue of the GI tract, such as when a submucosal tissue expansion procedure is performed in one segment of the GI tract after which an energy delivery to mucosal tissue is performed in that same segment. Catheter 200 can be constructed and arranged to treat multiple relatively contiguous segments (contiguous segments herein) or non-contiguous segments of the GI tract. In some embodiments, two or more axial segments of submucosal tissue of intestine are expanded, after which a single ablation procedure is performed (e.g. an ablation of a length of tissue of similar or lesser length as compared to the cumulative length of submucosal tissue expanded, such as when the length treated by a single ablation step is greater than the length expanded in a single tissue expansion step), such as is described hereinbelow in reference to FIGS. 1B-1C.

In some embodiments, system 10 comprises one or more body access devices, such as endoscope 50 shown. Catheter 200 can be configured to be inserted through one or more working channels of endoscope 50 and/or alongside endoscope 50. In some embodiments, catheter 200 is inserted through a sheath attached to endoscope 50. Catheter 200 can comprise a length such that it can be inserted through the patient's mouth and into one or more locations within the stomach, the duodenum, the jejunum and/or the ileum.

In some embodiments, system 10 comprises one or more guidewires, such as guidewire 60 shown. In these embodiments, catheter 200 can be advanced over guidewire 60, such as by using standard over-the-wire techniques, through one or more lumens of catheter 200.

Console 100 can include one or more conduits, conduit 191 configured to transport fluid to and/or from console 100. Console 100 can include pump assembly 195 that includes one or more pumps or other fluid delivery mechanisms (‘pump” herein) that deliver fluid (e.g. a liquid, a gas, and/or a gel) into one or more fluid pathways or other locations within catheter 200. Console 100 can include one or more reservoirs that store these fluids to be delivered. Alternatively or additionally, console 100 can be attachable to a fluid-storing reservoir separate from console 100 (or positioned in a second housing of console 100). Pump assembly 195 and/or another component of console 100 can include one or more pumps or other fluid removal mechanisms (“pump” herein) that extract fluid from one or more lumens or other locations within of catheter 200. Console 100 can include one or more reservoirs that store these removed fluids, or they can be stored in a reservoir separate from console 100 (or positioned in a second housing of console 100). Pump assembly 195 and/or another component of console 100 can include one or more pumps or other vacuum generating mechanisms (“pump” herein) that generate a vacuum that can cause a negative pressure within one or more lumens or other locations within catheter 200.

Console 100 can comprise one or more discrete components, such as one or more components each with a discrete (i.e. separate) housing that surround one or more pumps and/or reservoirs.

In some embodiments, console 100 comprises vacuum supply 110. Vacuum supply 110 can comprise one or more pumps configured to generate a vacuum within catheter 200 and/or other component of system 10. In some embodiments, vacuum supply 110 includes one or more reservoirs configured to reduce variations in vacuum pressure. Vacuum supply 110 can provide a vacuum to one, two, three or more ports configured to engage tissue, such as tissue capture chambers 510 described herein. Vacuum supply 110 can be configured to provide a vacuum pressure of between −2 psi and −14.7 psi, such as between −4 psi and −14.7 psi. In some embodiments, system 10 can be configured to operate with vacuum supply 110 providing a vacuum pressure of between −6 psi and −12.5 psi. Additionally or alternatively, vacuum supply 110 and/or another component of console 100 can comprise at least one sensor, such as a sensor-based functional element 199, configured to monitor the pressure of vacuum supply 110, and provide an alert (e.g. an alert to the operator and/or enter a system wide alert mode) if the vacuum pressure is insufficient or otherwise undesired (e.g. if the vacuum pressure is above or below a desired level, an expected level, and/or other threshold). In some embodiments, vacuum supply 110 provides an aspiration reservoir, such as to remove a fluid from locations proximate the distal end of catheter 200 (e.g. gas or other fluid within the gastrointestinal tract removed in a desufflation procedure and/or a fluid within a distal portion of catheter 200).

In some embodiments, console 100 comprises injectate fluid supply 120. Injectate supply 120 can comprise one or more pumps configured to deliver one or more injectates, injectate 125 shown, to catheter 200 and/or other component of system 10. In some embodiments, injectate supply 120 includes one or more reservoirs configured to store injectate 125. In some embodiments, injectate supply 120 comprise a pump (e.g. a syringe pump configured to drive 2, 3 or more syringes simultaneously or sequentially), such as a pump that is part of pump assembly 195. In some embodiments, injectate supply 120 comprises injectate 125. Injectate supply 120 can deliver fluid to one, two, three or more elements configured to deliver injectate 125 onto and/or into tissue, such as injectate delivery elements 520 described herein.

Injectate supply 120 can be configured to deliver fluid at a flow rate of at least 10 mL/min, such as at a flow rate of at least 15 mL/min, 20 mL/min, 40 mL/min, or 60 mL/min. In some embodiments, injectate supply 120 delivers fluid via two or more injectate delivery elements 520 simultaneously (e.g. in a tissue expansion procedure), at a rate of at least 10 mL/min per injectate delivery element 520, such as at a rate of at least 12.5 mL/min, 15 mL/min, 20 mL/min, 40 mL/min, or 60 mL/min per fluid delivery element. In some embodiments, injectate supply 120 is configured to deliver a volume between 2 mL and 20 mL (e.g. approximately 10 mL) to multiple injectate delivery elements 520 simultaneously (e.g. two, three or four injectate delivery elements 520 simultaneously) in a time period less than 60 seconds, less than 40 seconds, less than 30 seconds, less than 20 seconds, less than 10 seconds, and/or less than 5 seconds (e.g. in a tissue expansion procedure). Injectate supply 120 can be further configured to deliver fluid (e.g. to injectate delivery elements 520, conduits 521, and/or another component of system 10) at a pressure of at least 40psi, such as at a pressure of at least 75 psi, 100 psi, 200 psi, or 300 psi. Injectate supply 120 can be configured to provide a bolus of injectate 125 to two, three or more injectate delivery elements (simultaneously or sequentially) in order to expand an axial segment of submucosal tissue (e.g. a full or partial circumferential band of submucosal tissue with a length of at least 0.25 cm, at least 0.5 cm or at least 0.75 cm) to achieve an expansion of the submucosal layer to a thickness of at least 250 μm, or approximately 400 μm (e.g. in the area surrounding the volume of mucosal tissue to be subsequently ablated).

Injectate 125 can comprise one or more liquids, gels, and/or other flowable materials for injecting into tissue, such as to expand one or more layers of tissue (e.g. submucosal tissue expanded prior to a mucosal ablation procedure) and/or to narrow a lumen of the intestine and/or other segment of the GI tract (e.g. to create a therapeutic restriction). Alternatively or additionally, injectate 125 can comprise an agent configured to cause tissue necrosis.

Alternatively or additionally, injectate 125 can comprise a warming and/or cooling fluid delivered onto and/or into tissue (e.g. a neutralizing fluid such as neutralizing fluid 155 configured to limit, stop and/or at least reduce ablation performed by functional assembly 500). In some embodiments, injectate 125 comprises one, two or more materials selected from the group consisting of: a peptide polymer (e.g. a peptide polymer configured to stimulate fibroblasts to produce collagen); polylactic acid; polymethylmethacrylate (PMMA); a hydrogel; ethylene vinyl alcohol (EVOH); a material configured to polymerize EVOH; dimethyl sulfoxide (DMSO); saline; material harvested from a mammalian body; autologous material; fat cells; collagen; autologous collagen; bovine collagen; porcine collagen; bioengineered human collagen; dermis; a dermal filler; hyaluronic acid; conjugated hyaluronic acid; calcium hydroxylapatite; fibroblasts; a sclerosant; an adhesive; cyanoacrylate; a pharmaceutical agent; a visualizable material; a radiopaque material; a visible dye; ultrasonically reflective material; a combination of materials configured to cause an endothermic reaction when mixed (e.g. when mixed in tissue); a combination of materials configured to cause an exothermic reaction when mixed (e.g. when mixed in tissue); a combination of material configured to expand when mixed (e.g. when mixed in tissue); and combinations of one or more of these. In some embodiments, injectate 125 comprises beads (e.g. pyrolytic carbon-coated beads) suspended in a carrier (e.g. a water-based carrier gel). In some embodiments, injectate 125 comprises a solid silicone elastomer (e.g. heat-vulcanized polydimethylsiloxane) suspended in a carrier, such as a bio-excretable polyvinylpyrrolidone (PVP) carrier gel. In some embodiments, injectate 125 has an adjustable degradation rate, such as an injectate 125 comprising one or more cross linkers in combination with polyalkyleneimines at specific concentrations that result in hydrogels with adjustable degradation properties. In some embodiments, injectate 125 and/or agent 80 comprises living cells, such as living cells injected into the mucosa or submucosa of the intestine to provide a therapeutic benefit.

In some embodiments, injectate 125 comprises a visualizable and/or otherwise detectable (e.g. magnetic) material (e.g. in addition to one or more materials of above) selected from the group consisting of: a dye; a visible dye; indigo carmine; methylene blue; India ink;

SPOT™ dye; a visualizable media; radiopaque material; radiopaque powder; tantalum; tantalum powder; ultrasonically reflective material; magnetic material; ferrous material; and combinations of one or more of these.

In some embodiments, a volume of injectate 125 is delivered into tissue to create a therapeutic restriction (e.g. a therapeutic restriction with an axial length between 1 mm and 20mm), as described herein, and/or as is described in applicant's co-pending U.S. patent application Ser. No. 15/156,585, entitled “Systems, Devices and Methods for the Creation of a Therapeutic Restriction in the Gastrointestinal Tract”, filed May 17, 2016, the content of which is incorporated herein by reference in its entirety for all purposes. In some embodiments, a volume of injectate 125 is delivered into tissue to create a safety margin of tissue prior to an ablation procedure, as is described herein.

In some embodiments, injectate 125 comprises a fluorescent-labeled material or other biomarker configured to identify the presence of a biological substance, such as to identify diseased tissue and/or other tissue for treatment by functional assembly 500 (e.g. to identify target tissue). For example, injectate 125 can comprise a material configured to be identified by imaging device 70 (described below), such as to identify a visualizable change to injectate 125 that occurs after contacting one or more biological substances. In these embodiments, imaging device 70 can comprise a molecular imaging device, such as when imaging device 70 comprises a molecular imaging probe and injectate 125 comprises an associated molecular imaging contrast agent. In these embodiments, injectate 125 can be configured to identify diseased tissue and/or to identify a particular level of one or more of pH, tissue oxygenation, blood flow, and the like. Injectate 125 can be configured to be delivered onto the inner surface of intestinal or other tissue, and/or to be delivered into tissue (i.e. beneath the surface).

In some embodiments, console 100 comprises inflation fluid supply 130. Inflation fluid supply 130 can comprise one or more pumps configured to deliver one or more fluids, inflation fluid 135 shown, to inflate one or more portions of catheter 200 and/or other component of system 10. In some embodiments, inflation fluid supply 130 includes one or more reservoirs configured to store inflation fluid 135. In some embodiments, inflation fluid supply 130 comprises inflation fluid 135. Inflation fluid supply 130 can deliver inflation fluid 135 to a balloon or other reservoir (e.g. other fluid expandable component), such as expandable element 530 described herein. Inflation fluid supply 130 can be configured to deliver a bolus volume of fluid to expandable element 530, such as a bolus of between 0.1 mL and 12 mL, such as an operator selectable bolus volume of 6 mL, 8 mL, and/or 10 mL. Inflation fluid supply 130 can be configured to deliver fluid to expandable element 530 at a pressure of between 0.1 psi and 5 psi.

In some embodiments, inflation fluid supply 130 delivers fluid to expandable element 530 prior to a tissue expansion procedure as described herein, in which a separate fluid, injectate 135, is delivered directly into submucosal or other tissue via one, two or more injectate delivery elements 520. In these embodiments, the fluid provided to expandable element 530 by inflation fluid supply 130 can comprise inflation fluid 135 and/or a different fluid, such as neutralizing fluid 155. Neutralizing fluid 155 can be delivered to expandable element 530 in a submucosal expansion procedure, such as to provide the additional function of pre-cooling or pre-warming tissue proximate element 530 prior to a subsequent thermal ablation procedure (e.g. a heat ablation or cryogenic ablation, respectively, performed by element 530). Alternatively or additionally, inflation fluid supply 130 can deliver neutralizing fluid 155 to element 530 in a tissue expansion procedure performed shortly after a (previous) ablation procedure, such as to perform a post-cooling and/or post-warming of tissue configured to limit the effects of a heat ablation or cryogenic ablation, respectively. For example, pre or post-cooling, and/or pre or post-warming can be performed to reduce time in a previous and/or subsequent ablation step.

In some embodiments, console 100 comprises ablative fluid supply 140. Ablative fluid supply 140 can comprise one or more pumps configured to deliver one or more ablative fluids, ablative fluid 145 shown, to one or more portions of catheter 200 and/or other component of system 10. In some embodiments, ablative fluid supply 140 includes one or more reservoirs configured to store ablative fluid 145. In some embodiments, ablative fluid supply 140 comprises ablative fluid 145. Ablative fluid supply 140 can deliver ablative fluid 145 to a balloon and/or other fluid storing assembly and/or component of catheter 200, such as an ablative fluid reservoir (e.g. a balloon), expandable element 540 and/or another expandable element 530 described herein. Alternatively or additionally, ablative fluid supply 140 can deliver ablative fluid 145 to one, two, three or more fluid delivery elements configured to deliver fluid onto and/or within tissue, such as injectate delivery elements 520 described herein. Ablative fluid supply 140 can be configured to deliver ablative fluid at a flow rate of at least 5 mL/s, such as at least 8 mL/s, 9 mL/s, 10 mL/s, 15 mL/s, and/or 20 mL/s. In some embodiments, catheter 200 comprises a hydraulic inflow resistance (resistance to ablative fluid supply 140 and/or another fluid supply described herein) of less than 0.05 psi/(mL/min), such as less than 0.036 psi/(mL/min) (e.g. when measured at 85° C. at a flow rate of 570 mL/min). In some embodiments, catheter 200 comprises a hydraulic inflow resistance of at least 0.020 psi/(mL/min), such as at least 0.030 psi/(mL/min) (e.g. when measured at 85° C. at a flow rate of 570 mL/min). In some embodiments, catheter 200 comprises a hydraulic outflow resistance less than 0.070 psi/(mL/min), such as less than 0.6 3psi/(mL/min) (e.g. when measured at 85° C. at a flow rate of 570 mL/min). In some embodiments, catheter 200 comprises a hydraulic outflow resistance of at least 0.040 psi/(mL/min), such as at least 0.53 psi/(mL/min) (e.g. when measured at 85° C. at a flow rate of 570 mL/min). Additionally or alternatively, ablative fluid supply 140 can be configured to deliver ablative fluid at a pressure of approximately 40 psi (pressure leaving console 100), such that the pressure of the ablative fluid within expandable element 530 is approximately 20 psi. In some embodiments, ablative fluid supply 140 provides fluid at an ablative temperature (e.g. sufficiently hot or sufficiently cold temperature) in a recirculating manner.

In some embodiments, console 100 comprises neutralizing fluid supply 150.

Neutralizing fluid supply 150 can comprise one or more pumps configured to deliver one or more neutralizing fluids, neutralizing fluid 155 shown, to one or more portions of catheter 200 and/or other component of system 10 (e.g. a fluid configured to neutralize ablative effects of an ablative fluid delivered by ablative fluid supply 140). In some embodiments, neutralizing fluid supply 150 includes one or more reservoirs configured to store neutralizing fluid 155. In some embodiments, neutralizing fluid supply 150 comprises neutralizing fluid 155. Neutralizing fluid supply 150 can deliver neutralizing fluid 155 to a balloon and/or other fluid storing assembly or component of catheter 200, such as a neutralizing fluid reservoir, expandable element 550, expandable element 540, and/or other expandable element 530 described herein. Alternatively or additionally, neutralizing fluid supply 150 can deliver neutralizing fluid 155 to one, two, three or more fluid delivery elements configured to deliver fluid onto and/or within tissue, such as injectate delivery elements 520 described herein. Neutralizing fluid supply 150 can be configured to deliver neutralizing fluid at a flow rate of at least 5 mL/s, such as at least 8 mL/s, 9 mL/s, 10 mL/s, 15 mL/s, or 20 mL/s. Additionally or alternatively, neutralizing fluid supply 150 can be configured to deliver neutralizing fluid at a pressure of approximately 40 psi (pressure leaving console 100), such that the pressure of the neutralizing fluid within expandable element 530 is approximately 20 psi. In some embodiments, ablative fluid 145 provided by ablative fluid supply 140 is delivered to a fluid storing component of catheter 200 (e.g. expandable element 530) and neutralizing fluid 155 provided by neutralizing fluid supply 150 is delivered onto and/or within tissue (e.g. via one or more injectate delivery elements 520). Alternatively or additionally, ablative fluid 145 provided by ablative fluid supply 140 can be delivered onto and/or within tissue (e.g. via one or more injectate delivery elements 520), while neutralizing fluid 155 provided by neutralizing fluid supply 150 is delivered to a balloon and/or other fluid storing assembly or component of catheter 200, such as expandable element 530, expandable element 540, and/or expandable element 550 described herein. In some embodiments, ablative fluid supply 140 comprises neutralizing fluid supply 150 (e.g. a single assembly comprising one or more pumps that provide both ablative fluid 145 and neutralizing fluid 155 from one, two or more reservoirs).

In some embodiments, inflation fluid supply 130, ablative fluid supply 140, neutralizing fluid supply 150 and/or another fluid delivery assembly of console 100 is configured to provide fluid (e.g. inflation fluid 135, ablative fluid 145 and/or neutralizing fluid 155) to functional assembly 500 (e.g. to one or more expandable elements 530) at a flow rate of at least 2 mL/sec, such as at least 5 mL/sec, or at a flow rate of approximately 9.5 mL/sec. In some embodiments, console 100 provides fluid to functional assembly 500 at a flow rate of no more than 30 mL/sec.

In some embodiments, console 100 comprises fluid removal pump 160. Fluid removal pump 160 can comprise one or more pumps configured to remove fluid from one or more portions of catheter 200 or other component of system 10. In some embodiments, fluid removal pump 160 includes one or more reservoirs configured to store the one or more removed fluids. In some embodiments, fluid removed by fluid removal pump 160 is recirculated to one or more other assemblies of console 100, such as inflation fluid supply 130, ablative fluid supply 140, neutralizing fluid supply 150, insufflation supply 170 (described hereinbelow) and/or hydraulic fluid supply 180 (also described hereinbelow). Fluid removal pump 160 can remove fluid from a balloon or other fluid storing assembly or component of catheter 200, such as expandable element 530, expandable element 540, and/or expandable element 550 described herein. In some embodiments, fluid removal pump 160 is configured to remove (e.g. from catheter 200 and/or any component of system 10) injectate 125, inflation fluid 135, ablative fluid 145, neutralizing fluid 155, insufflation fluid 175, and/or hydraulic fluid 185, each as described herein. In some embodiments, catheter 200 comprises a hydraulic outflow resistance as described hereinabove in reference to ablation fluid supply 140. In some embodiments, ablative fluid supply 140 and/or neutralizing fluid supply 150 comprise fluid removal pump 160. In some embodiments, pump assembly 195 comprises fluid removal pump 160.

In some embodiments, console 100 comprises insufflation supply 170. Insufflation supply 170 can comprise one or more pumps configured to deliver a gas or other insufflation fluid, insufflation fluid 175 shown, to inflate the duodenum or other segment of the patient's GI tract. Alternatively or additionally, insufflation supply 170 can be configured to remove insufflation fluid 175 and/or other fluid from the duodenum or other segment of the patient's GI tract (i.e. perform a desufflation). In some embodiments, insufflation supply 170 includes one or more reservoirs configured to store insufflation fluid 175 (to be provided and/or recently removed). In some embodiments, insufflation supply 170 comprises insufflation fluid 175. Insufflation supply 170 can deliver and/or remove fluids via catheter 200 and/or a separate component of system 10, such as an endoscope or other body access device, endoscope 50.

In some embodiments, console 100 comprises functional fluid supply 180. Functional fluid supply 180 can provide functional fluid 185 to one or more components or assemblies of catheter 200 and/or other component of system 10. In some embodiments, functional fluid 185 comprises a hydraulic or pneumatic fluid (“hydraulic fluid” herein). In some embodiments, functional fluid 185 comprises a conductive fluid, such as a fluid configured to transmit electrical power and/or electrical signals between functional assembly 500 and console 100.

As described hereabove, console 100 can comprise one or more pumps, pump assembly 195. Pump assembly 195 can be configured to deliver and/or extract fluids from catheter 200 (e.g. with or without an intermediate connection device such as umbilical 600 described herebelow). In some embodiments, pump assembly 195 is fluidly attached to at least injectate supply 120 and/or inflation supply 130, such as to supply injectate 125 and/or inflation fluid 135, respectively, to catheter 200. In some embodiments, pump assembly 195 is fluidly attached to injectate supply 120, inflation fluid supply 130, ablative fluid supply 140, neutralizing fluid supply 150, insufflation supply 170, and/or functional fluid supply 180, such as to deliver and/or remove their associated fluids to and/or from catheter 200. In some embodiments, one or more of injectate supply 120, inflation fluid supply 130, ablative fluid supply 140, neutralizing fluid supply 150, insufflation supply 170, and/or functional fluid supply 180 comprise one or more pumps integrated into their assembly (e.g. one or more pumps of pump assembly 195 are integrated into the supply). In some embodiments, pump assembly 195 is configured as described herebelow in reference to FIG. 1A.

Console 100 comprises one or connectors, connector 102 shown, which fluidly connects to one or more of assemblies 110, 120, 130, 140, 150, 160, 170, and/or 180 of console 100 described herein, via conduits 111, 121, 131, 141, 151, 161, 171, and/or 181, respectively. In some embodiments, console 100 comprises pump assembly 195, which fluidly connects conduits 111, 121, 131, 141, 151, 161, 171, and/or 181 to connector 102 via one or more other conduits, such as conduit 191 shown. Alternatively or additionally, console 100 can comprise one or more manifolds, manifold 700a shown, which fluidly connects conduits 111, 121, 131, 141, 151, 161, 171, and/or 181 to connector 102 via one or more other conduits, such as conduit 191 shown. Alternatively, conduits 111, 121, 131, 141, 151, 161, 171, and/or 181 directly attach to connector 102 (i.e. without pump assembly 195 and/or without manifold 700a). Manifold 700a can be constructed and arranged to fluidly combine one or more of conduits 111, 121, 131, 141, 151, 161, 171 and/or 181. Alternatively or additionally, manifold 700a can be constructed and arranged to split (divide) one or more of conduits 111, 121, 131, 141, 151, 161, 171, and/or 181 into multiple conduits. In some embodiments, manifold 700a includes one or more valves configured to control flow of fluid in a conduit. In some embodiments, manifold 700a includes one or more sensors (e.g. temperature and/or pressure sensors) configured to provide a signal related to a parameter (e.g. temperature and/or pressure) of fluid within a conduit.

In some embodiments, system 10 comprises a connecting device, umbilical 600 which operably connects (e.g. at least fluidly connects) catheter 200 to console 100. Alternatively or additionally, catheter 200 can attach directly to console 100 (e.g. connector 102 of console 100 attaches directly to connector 302 of catheter 200). Umbilical 600 comprises one or more proximal connectors, connector 602a shown, which operably attaches to mating connector 102 of console 100. Umbilical 600 comprises one or more distal connectors, connector 602b shown, which operably attaches to mating connector 302 of handle assembly 300 of catheter 200. Umbilical 600 can comprise one or more fluid delivery tubes or other fluid-transporting conduits, conduit 691 shown. Conduit 691 comprises one or more lumens or other conduits configured to allow passage of one or more similar and/or dissimilar fluids between console 100 and catheter 200. Each conduit can be configured to receive one or more shafts or other conduits which transport one or more fluids. In some embodiments, umbilical 600 further comprises one or more of: wires or other electrical filaments configured to transmit electrical power and/or signals; optical fibers or other conduits configured to transmit optical power and/or signals; waveguides or other sound conduits configured to transmit sonic power and/or signals; mechanical linkages (e.g. translatable rods); and/or other elongate structures configured to transmit energy, signals, and/or mechanical motion between console 100 and catheter 200. In some embodiments, umbilical 600 comprises one or more sensors, transducers, and/or other functional elements, such as functional element 699 described herebelow. Functional element 699 can be positioned proximate conduit 691 as shown, positioned proximate connector 602a, and/or positioned proximate connector 602b.

Catheter 200, including distal portion 208 and distal end 209, comprises handle assembly 300, shaft assembly 400, and functional assembly 500. Handle assembly 300 is positioned on the proximal end or at least a proximal portion of shaft assembly 400, and functional assembly 500 is positioned on catheter 200 distal portion 208 (e.g. on the distal end or at least a distal portion of shaft assembly 400).

Shaft assembly 400 includes at least one elongate shaft assembly, shaft 401, which comprises one or more lumens or other conduits, conduit 491, each of which can be configured to attach to one or more conduits of handle 300, conduit 391. In some embodiments, one or more conduits of conduit 491 simply passes through handle 300 (e.g. to operably attach to umbilical 600 and/or console 100). Each conduit of shaft 401 can be configured to transport fluid and/or it can be sized to receive (e.g. slidingly receive) one or more separate shafts, such as one or more shafts that transport fluid. In some embodiments, on or more lumens of shaft 401 receive a separate shaft, and fluid is transported within the received shaft and/or between the outer diameter of the received shaft and the wall of the lumen of shaft 401, such as is described herebelow. Alternatively or additionally, each lumen of shaft 401 and/or one or more shafts inserted within the lumen can surround (e.g. slidingly or fixedly surround) one or more conduits configured to transmit energy, signals, and/or mechanical motion between console 100 and catheter 200, as described herein.

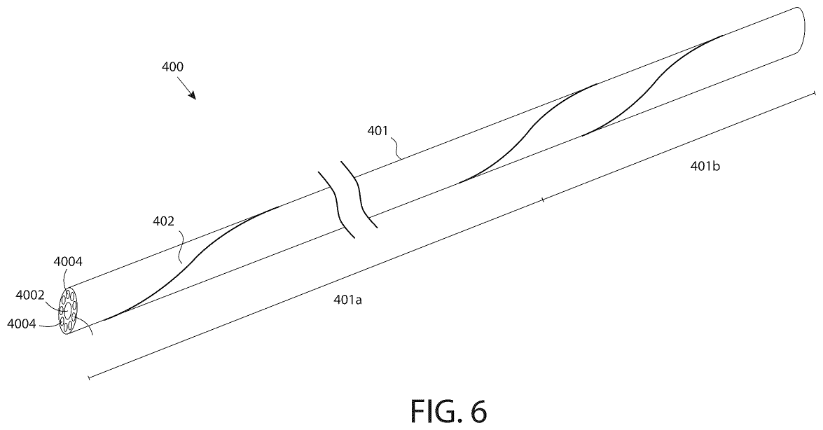

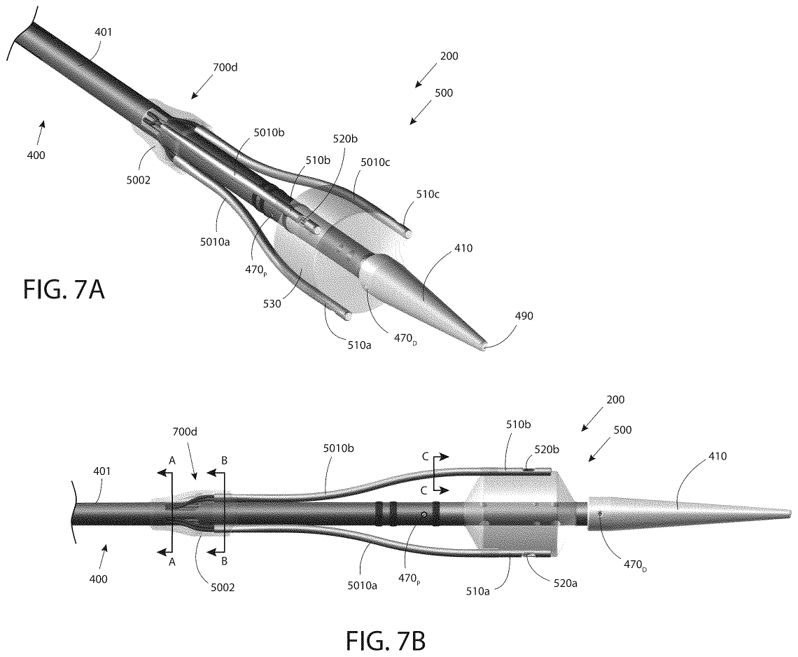

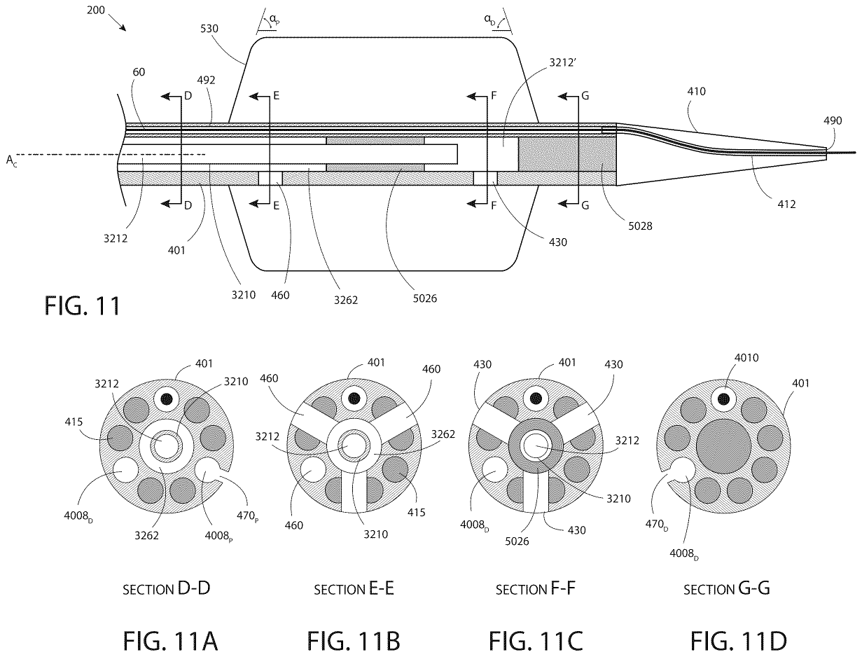

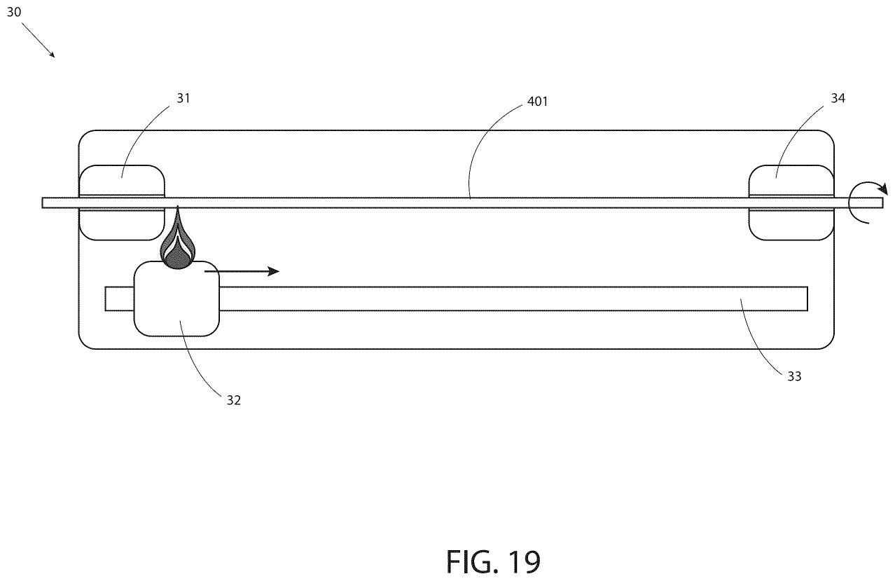

Shaft assembly 400 comprises proximal end 405, proximal portion 406, middle portion 407, distal portion 408, and distal end 409. Distal portion 408 is shown in a magnified view. Positioned on distal portion 408 is functional assembly 500, configured as a treatment assembly and/or diagnostic assembly (e.g. an assembly configured to treat and/or diagnose tissue of the intestine or other GI tract tissue). In some embodiments, shaft 401 extends through and beyond functional assembly 500 (as shown in FIG. 1, where catheter 200 distal end 209 is the same as shaft assembly 400 distal end 409). Alternatively, functional assembly 500 can be positioned on the distal end of shaft 401. In some embodiments, shaft 401 comprises a twist, such as is described herebelow in reference to FIGS. 6, 19 and/or 20. In some embodiments, shaft 401 comprises a bulbous tip. In some embodiments, shaft 401 comprises a tapered tip, such as is described herebelow in reference to FIGS. 7A, 7B and 11.

In some embodiments, shaft assembly 400 comprises a lumen to slidingly receive a guidewire, such as a passageway including a lumen (e.g. lumen 412 described herebelow in reference to FIG. 11 and/or other lumens of shaft assembly 400) which exits at a location proximate the distal end 409 of shaft assembly 400 at an opening, port 490. In some embodiments, shaft assembly 400 comprises one or more lumens for performing insufflation and/or desufflation (“insufflation” herein), such as conduit 571 comprising one or more lumens which terminate in one or more openings, such as port 470_{D} positioned distal to functional assembly 500 and port 470p positioned proximal to functional assembly 500, each as shown and described herein. In some embodiments, port 470p and/or port 470p is configured to perform desufflation only, or insufflation only.

In some embodiments, shaft assembly 400 comprises one or more manifolds, manifold 700c shown, which fluidly connects one or more conduits of conduit 491 to one or more other conduits (e.g. one or more other conduits of conduit 491 or one or more other conduits of catheter 200). Manifold 700c can be constructed and arranged to fluidly combine one or more of lumens of conduit 491. Alternatively or additionally, manifold 700c can be constructed and arranged to split (divide) one or more of lumens of conduit 491 into multiple lumens. In some embodiments, manifold 700c includes one or more valves (e.g. one or more one-way valves) configured to control flow of fluid in a conduit. In some embodiments, manifold 700c includes one or more sensors (e.g. temperature and/or pressure sensors) configured to provide a signal related to a parameter (e.g. temperature and/or pressure) of fluid within a conduit.

In some embodiments, shaft assembly 400 comprises one or more sensors, transducers, and/or other functional elements, such as functional element 499a (e.g. positioned in a mid-portion of shaft 401 and/or proximate manifold 700c) and/or functional element 499b (e.g. positioned proximate manifold 700d and/or functional assembly 500) as shown and described herebelow. In some embodiments, functional element 499a and/or 499b comprises a radiopaque marker and/or other visualizable marker, as described herein, configured to allow an operator to visualize translation and/or rotation of shaft assembly 400 (e.g. to visualize translation and/or rotation of functional assembly 500), such as via imaging device 70 (e.g. a fluoroscope or other imaging device).

Shaft 401 can comprise a length of at least 60″, such as at least 72″. In some embodiments, shaft 401 comprises an outer diameter of less than 0.3″, such as a diameter less than 0.256″, 0.1″, or 0.08″. Shaft 401 can comprise a material selected from the group consisting of: a polyether block amide such as Pebax™; a thermoplastic elastomer, such as Tygon™, Arnitel™, or HytreI™; and combinations of one or more of these. In some embodiments, at least a portion of shaft 401 comprises a radiopaque additive, such as barium sulfate. In some embodiments, at least a portion of shaft 401 comprises a lubricious coating or additive, such as Propell™ low friction compound manufactured by Foster Corporation of Putnam, CT.

Functional assembly 500 comprises one or more assemblies configured to treat and/or diagnose tissue. In some embodiments, functional assembly 500 is configured to both treat and diagnose tissue. Functional assembly 500 can be configured to treat and/or diagnose duodenal tissue or other tissue of the GI tract. Functional assembly 500 can be positioned on distal portion 408 of shaft assembly 400 as shown. Functional assembly 500 can be configured to radially expand and/or radially contract, such as when functional assembly comprises one or more expandable reservoirs, such as one or more of expandable elements 530, 540 and/or 550 shown (singly or collectively, expandable element 530). Each expandable element 530, 540 and/or 550 (singly or collectively expandable element 530) can comprise a balloon or other expandable reservoir (“balloon” herein), an expandable cage, a furlable element, and the like. Expandable element 530 can comprise one or more balloons that circumferentially surround shaft 401 (e.g. in a linear arrangement), or multiple partially circumferential balloons (e.g. in a radial arrangement). Expandable elements 530 can comprise one or more balloons that expand radially out from shaft 401, at the same or different axial locations along shaft 401. An expandable element 530 can comprise an array of balloons in a lobed configuration, circumferentially spaced. An expandable element 530 can comprise one or more inner balloons 540 surrounded by one or more outer balloons 550 (e.g. where the inner balloon receives a first fluid at a first temperature and the space between the inner and outer balloons receives a second fluid at a second temperature, different than the first temperature). Expandable element 530 can comprise a balloon or other element configured to expand to a diameter of less than or equal to 35 mm, such as less than or equal to 30 mm or 25 mm. Expandable element 530 can comprise a material selected from the group consisting of: a compliant material; a non-compliant material; both a compliant and a non-compliant material; polyethylene terephthalate (PET); nylon; and combinations of one or more of these. Expandable element 530 can comprise a balloon with a wall thickness of at least 0.00025″, such as at least 0.00035″, at least 0.00050″, or a thickness of approximately 0.00075″. In some embodiments, one or more portions of expandable element 530 comprise a non-compliant material and one or more other portions of expandable element 530 comprises a compliant material. In some embodiments, expandable element 530 is configured to withstand an inflation pressure of up to 50 psi. In some embodiments, a first expandable element 530 comprises at least a portion comprising a non-compliant material and a second expandable element 540 comprises at least a portion comprising a compliant material.

Functional assembly 500 can comprise one or more balloons configured to receive one or more fluids, such as an expandable element 540 configured to receive an ablative fluid (e.g. a fluid at an ablative temperature received from ablative fluid supply 140), an expandable element 550 configured to receive a neutralizing fluid (e.g. a fluid received from neutralizing fluid supply 150 and comprising a temperature configured to cool or warm tissue after a heat or cryogenic ablation, respectively), or other expandable element 530. In some embodiments, at least expandable element 540 and expandable element 550 are the same reservoir (e.g. the same one or more balloons) that receive both ablative fluid and neutralizing fluid.

In some embodiments, functional assembly 500 is configured to expand one or more layers of tissue, such as to expand one or more layers of submucosal tissue prior to a tissue treatment procedure in which a mucosal layer of tissue is treated (e.g. thermally or chemically ablated). In these embodiments, functional assembly 500, catheter 200 and/or any component of system 10 can be of similar construction and arrangement to that described in: applicant's co-pending U.S. patent application Ser. No. 14/515,324 , entitled “Tissue Expansion Devices, Systems and Methods”, filed Oct. 15, 2014; applicant's co-pending U.S. patent application Ser. No. 15/156,585 , entitled “Systems, Devices and Methods for the Creation of a Therapeutic Restriction in the Gastrointestinal Tract”, filed May 17, 2016; and applicant's co-pending U.S. patent application Ser. No. 15/274,948 , entitled “Injectate Delivery Devices, Systems and Methods”, filed Sep. 23, 2016; the content of each of which is incorporated herein by reference in its entirety for all purposes.

In some embodiments, functional assembly 500 is configured to receive an ablative fluid (e.g. a recirculating hot or cold fluid at a tissue-ablating temperature) to treat tissue. In some embodiments, functional assembly 500 is configured to deliver an ablation fluid directly onto tissue (e.g. a hot or cold liquid or gas at a tissue-ablating temperature, and/or a chemically ablative fluid). In these embodiments, functional assembly 500, catheter 200 and/or any component of system 10 can be of similar construction and arrangement to that described in: applicant's co-pending U.S. patent application Ser. No. 14/470,503 , entitled “Heat Ablation Systems, Devices and Methods for the Treatment of Tissue”, filed Aug. 27, 2014; and applicant's co-pending U.S. patent application Ser. No. 14/917,243 , entitled “Systems, Methods and Devices for Treatment of Target Tissue”, filed Mar. 7, 2016; the content of each of which is incorporated herein by reference in its entirety for all purposes.

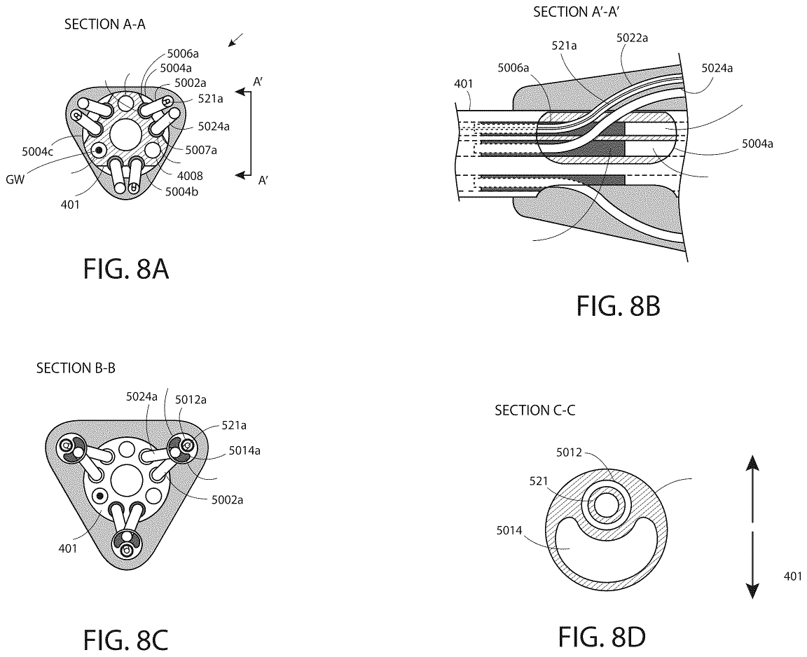

Functional assembly 500 can include one or more ports configured to capture and/or engage tissue (singly or collectively “capture” or “engage” herein) or otherwise stabilize functional assembly 500 within a GI lumen, such as tissue capture chambers 510 shown and described herein. Each tissue capture chamber 510 includes an opening, opening 512. In some embodiments, functional assembly 500 (or another portion of catheter 200) includes two, three, four or more tissue capture chambers 510. Each tissue capture chamber 510 can be attached to a source of vacuum, such as conduit 511 which is fluidly attached to a source of vacuum provided by console 100, such as vacuum supply 110. Each tissue capture chamber 510 can be of similar construction and arrangement to any chamber 510 described herebelow. In some embodiments, injectate delivery element 520 is positioned above (radially out from) a source of vacuum that is provided to tissue capture chamber 510, as shown in FIG. 8D. In some embodiments, one or more tissue capture chambers 510 is constructed of a metal or other material with a relatively high thermal conductance, such as to efficiently transfer heat from and/or to expandable element 530 (e.g. from and/or to temperature-ablative fluid within expandable element 530), such as to avoid non-ablated tissue regions proximate tissue capture chambers 510.

Functional assembly 500 can comprise one or more elements configured to deliver fluid into tissue, such as injectate delivery elements 520 shown, each positioned within or at least proximate a tissue capture chamber 510. In some embodiments, functional assembly 500 (or another portion of catheter 200) includes two, three, four or more injectate delivery elements 520. Injectate delivery elements 520 can comprise one or more elements selected from the group consisting of: needle; fluid jet; iontophoretic element; and combinations of one or more of these. Each injectate delivery element 520 can be operably attached to one or more conduits of catheter 200, such when fluidly connected to conduit 521 shown or when fluidly attached to a separate conduit slidingly received by conduit 521 as described herebelow. Each injectate delivery element 520 can be connected to a source of fluid, such as a fluid provided by console 100 via injectate supply 120, ablative fluid supply 140, neutralizing fluid supply 150, and/or functional fluid supply 180. One or more injectate delivery elements 520 can comprise a needle with a diameter between 16 gauge and 34 gauge, such as a needle with a 27 gauge or 29 gauge diameter One or more injectate delivery elements 520 can comprise a needle with a bevel angle of approximately 10°(e.g. with a bevel length of 0.008″), such as a bevel angle of at least 5°and/or a bevel angle no more than 45° or no more than 80°. One or more injectate delivery elements 520 can be advanced into the tissue contained in the associated tissue capture chambers 510, while avoiding the potential of the injectate delivery elements 520 penetrating an outer layer and/or outside of the GI wall tissue (e.g. injectate delivery elements 520 do not exit chambers 510). In some embodiments, tissue is penetrated by a needle-based injectate delivery element 520 at the time of the application of the vacuum to chamber 510, without the advancement of injectate delivery elements 520 (e.g. when the distal end of each injectate delivery element 520 is positioned within the associated chamber 510). In some embodiments, one or more injectate delivery elements 520 comprises a fluid jet, and injectate 125 or other fluid can be delivered into tissue captured within chamber 510 without advancement of the water jet. Each tissue capture chamber 510 can be configured to slidingly receive an injectate delivery element 520 (e.g. at a time in which tissue is captured within chamber 510 and the injectate delivery element 520 penetrates the captured tissue upon advancement), such as when a tissue capture chamber 510 is configured to slidingly receive at least a 29 gauge needle, or at least a 27 gauge needle. Each injectate delivery element 520 can be configured to be advanced a distance of at least 2.5 mm, at least 3.5 mm, or at least 4.5 mm. Each tissue capture chamber 510 can comprise a width of at least 0.010″, at least 0.040″ or at least 0.060″. Each tissue capture chamber 510 can comprise a width of no more than 0.25″, or no more than 0.35″. Each tissue capture chamber 510 can comprise a length of at least 0.010″, at least 0.040″ or at least 0.060″. Each tissue capture chamber 510 can comprise a length of no more than 0.9″, no more than 0.7″, or no more than 0.5″. Each tissue capture chamber 510 can comprise a depth of at least 300 μm, at least 500 μm, or at least 700 μm. Each tissue capture chamber 510 can comprise a depth of no more than 1500 μm.

Functional assembly 500 of FIG. 1 can comprise two tissue capture chambers 510 (e.g. separated circumferentially at approximately 180°) or three tissue capture chambers 510 (e.g. separated circumferentially at approximately 120° with the third chamber 510 hidden behind expandable element 530 as is shown in FIG. 7A), and each can surround a injectate delivery element 520. In some embodiments, four or more tissue capture chambers 510 are included. Each tissue capture chamber 510 can be configured to engage with tissue, such as to maintain contact between functional assembly 500 and tissue (e.g. during delivery and/or removal of energy to and/or from tissue). Alternatively or additionally, tissue capture chamber 510 can be configured to capture tissue within tissue capture chamber 510, via application of a vacuum, as described herein, such as to allow delivery of fluid or a fluid delivery element (e.g. a needle) into the captured tissue.

Functional assembly 500 can comprise one or more ports (e.g. openings) in shaft assembly 400 that are configured to deliver fluid into and/or remove fluid from expandable element 530, such as ports 430 and 460 shown. In some embodiments, ports 430 and 460 are constructed and arranged as described here below in reference to FIGS. 11 and 11A-D. Ports 430 and 460 can be positioned in various locations within expandable element 530. In some embodiments, port 460 is configured to remove fluid from expandable element 530, and is positioned in a proximal portion of functional assembly 500. In some embodiments, port 430 is configured to deliver fluid into expandable element 530, and can be positioned in a distal (as shown), middle or proximal portion of functional assembly 500. Port 430 can comprise one or more openings which are fluidly attached to one or more conduits, such as conduits 531, 541, and/or 551 as shown, which are fluidly connected to one or more of inflation fluid supply 130, ablative fluid supply 140 and/or neutralizing fluid supply 150, respectively, or other fluid supply of console 100 (e.g. functional fluid supply 180). Port 460 can comprise one or more openings fluidly connected to one or more conduits, such as conduit 561 as shown, which is fluidly connected to fluid removal pump 160 of console 100. In some embodiments, port 460 is fluidly attached to conduits 531, 541, and/or 551, which are fluidly connected to one or more of inflation fluid supply 130, ablative fluid supply 140 and/or neutralizing fluid supply 150, respectively, or other fluid supply of console 100 (e.g. functional fluid supply 180).

In some embodiments, functional assembly 500 comprises one or more sensors, transducers, and/or other functional elements, such as functional element 599 shown and described herebelow. In some embodiments, functional element 599 comprises a radiopaque marker and/or other visualizable marker, as described herein, configured to allow an operator to visualize translation and/or rotation of functional assembly 500, such as via imaging device 70 (e.g. a fluoroscope or other imaging device). In some embodiments, functional element 599 comprises a heat-generating transducer, such as an element comprising one, two, or more electrodes through which radiofrequency (RF) energy is passed, such as to heat expandable element 530, 540, and/or 550, and/or to heat fluid (e.g. saline) contained within expandable element 530, 540, and/or 550. Alternatively or additionally, functional element 599 can comprise a cooling transducer (e.g. a Peltier cooling element), such as to cool expandable element 530, 540, and/or 550, and/or to cool fluid contained within expandable element 530, 540, and/or 550.

Handle assembly 300 comprises a handle for an operator to manipulate catheter 200, including housing 301. Handle assembly 300 can be positioned in proximal end 405 of shaft assembly 400 as shown. Handle assembly 300 comprises one or more conduits, conduit 391. Conduit 391 can be configured to operably attach (e.g. on its proximal end or ends) to connector 102 of console 100 or to conduit 691 of umbilical 600. Conduit 391 is configured to operably attach (e.g. on its distal end or ends) to conduit 491 of shaft assembly 400. In some embodiments, handle assembly 300 comprises one or more manifolds, manifold 700b shown, which fluidly connects one or more conduits of conduit 391 to one or more other conduits (e.g. one or more other conduits of conduit 391 and/or conduit 491). Manifold 700b can be constructed and arranged to fluidly combine one or more of lumens of conduit 391. Alternatively or additionally, manifold 700b can be constructed and arranged to split one or more of lumens of conduit 391 into multiple lumens. In some embodiments, manifold 700b includes one or more valves configured to control flow of fluid in a conduit. In some embodiments, manifold 700b includes one or more sensors (e.g. temperature and/or pressure sensors) configured to provide a signal related to a parameter (e.g. temperature and/or pressure) of fluid within a conduit.

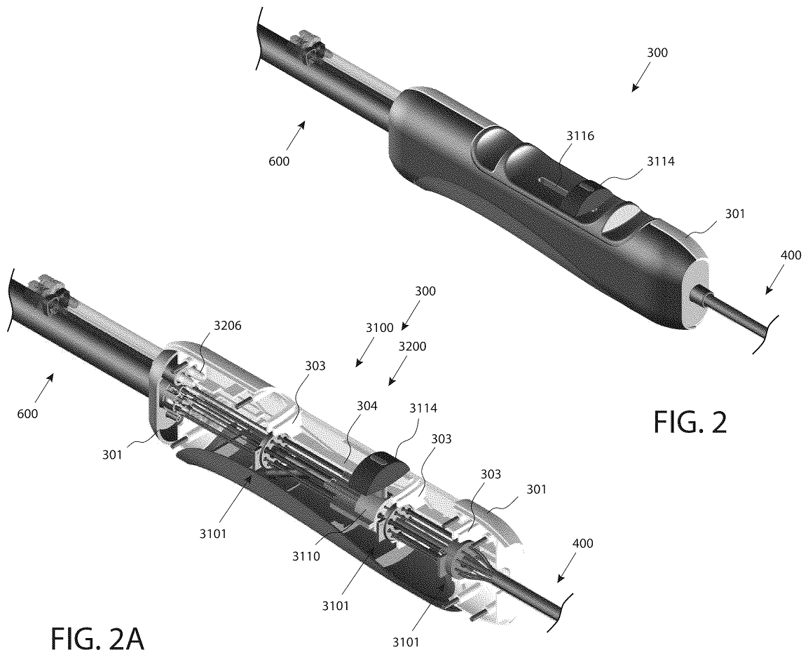

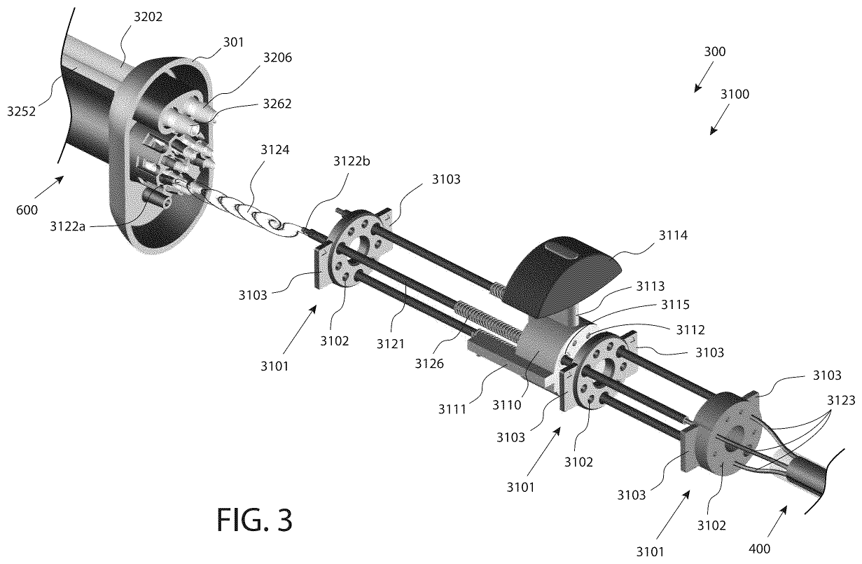

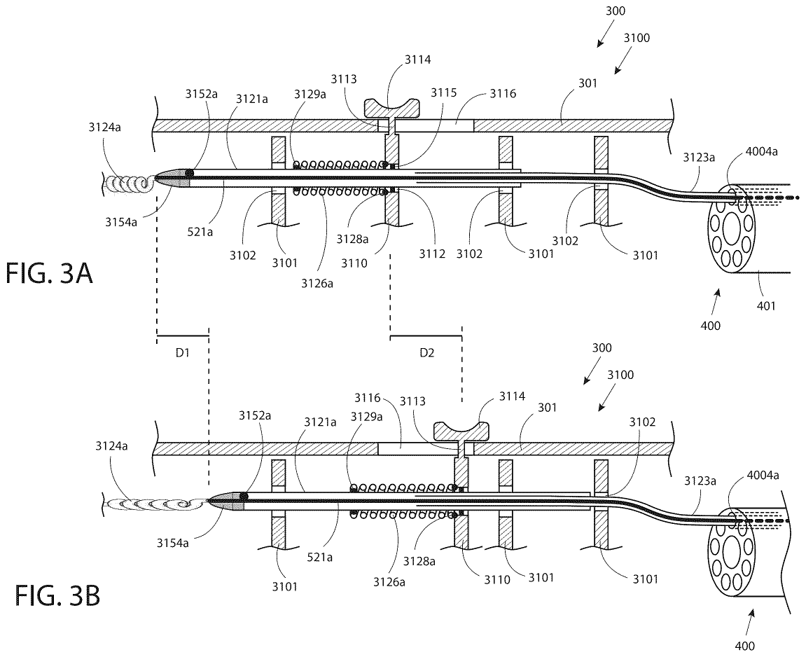

Handle assembly 300 can include one or more controls, control 310, which can be configured to activate, manipulate and/or otherwise operate one or more functions of catheter 200. In some embodiments control 310 comprises a control for advancing and/or retracting one or more injectate delivery elements 520 (e.g. simultaneously advancing and/or retracting two, three or more injectate delivery elements 520), such as via one or more compensation mechanisms described herebelow in reference to FIGS. 2, 2A, 3, 3A and/or 3B. In some embodiments, control 310 is configured to adjust one or more operating parameters of console 100 (e.g. via a wired or wireless connection).

Handle assembly 300 can include an entry port, such as port 392, for passage of a guidewire or other filament, such as guidewire 60. In some embodiments, port 392 is positioned on a proximal portion of shaft assembly 400. Port 392 can be operably connected to a lumen of shaft 401, such as is described herein.

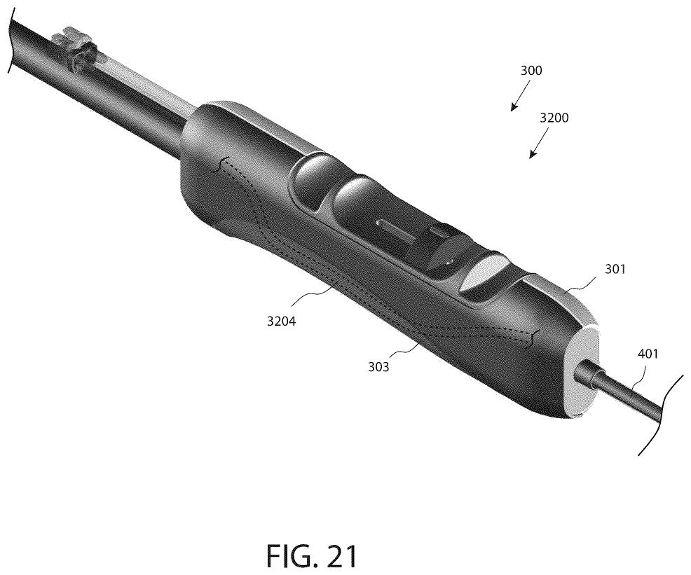

In some embodiments, handle assembly 300 comprises one or more sensors, transducers, and/or other functional elements, such as functional element 399 shown and described herebelow. In some embodiments, functional element 399 comprises a tactile transducer configured to alert an operator of a particular state of catheter 200 (e.g. an alarm or warning state, a “ready” state, a “function completed” state, and the like). For example, functional element 399 can alert an operator that a particular function is being performed, such as a function selected from the group consisting of: heating of tissue is being performed (e.g. via hot fluid present in functional assembly 500); a cooling of tissue is being performed (e.g. via cold fluid present in functional assembly 500); injectate is being delivered into tissue (e.g. injectate 125 is being delivered into submucosal or other tissue via one, two, three or more injectate delivery elements 520); needles or other injectate delivery elements 520 have been advanced into tissue; and combinations of one or more of these. Functional element 399 can comprise a tactile transducer selected from the group consisting of: a vibrational transducer (e.g. a vibrational transducer that alerts an operator that injectate is being delivered into tissue and/or injectate delivery elements 520 are presently advanced into tissue); a heating element (e.g. a heating element that alerts an operator that a heat ablation and/or warming of tissue is in process); a Peltier element or other cooling element (e.g. a cooling element that alerts an operator that a cryogenic ablation and/or cooling of tissue is in process); and combinations of one or more of these. In some embodiments, handle assembly 300 includes a portion configured to alert an operator of catheter 200 of one or more particular functional states of catheter 200, such as is described herebelow in reference to FIG. 21.

One or more functional elements can be included in system 10, such as functional element 199 of console 100, functional element 399 of handle assembly 300, functional element 499a and/or 499b of shaft assembly 400, functional element 599 of functional assembly 500, and/or functional element 699 of umbilical 600.

Endoscope 50 can comprise one or more endoscopes configured to reach at least one or more portions of the duodenum from the patient's mouth. In some embodiments, endoscope 50 comprises an endoscope similar to Olympus model number PCF-PH190.

Guidewire 60 can comprise an outside diameter of approximately 0.035″. Guidewire 60 can comprise a “stiff” or “super stiff” configuration, such as a guidewire similar to a Jagwire Stiff Straight guidewire, a Wallstent Super Stiff guidewire, a Dreamwire Superstiff, and/or a Savary Gilliard guidewire. Guidewire 60 can comprise a length of at least twice the length of catheter 200 and/or endoscope 50, such that one or more devices can be “exchanged” over guidewire 60. Guidewire 60 can comprise a material selected from the group consisting of: nitinol; stainless steel; and combinations of one or more of these. Guidewire 60 can comprise a hydrophilic or other lubricious coating, such as a Teflon coating.

In some embodiments, system 10 further comprises imaging device 70, which can comprise an imaging device constructed and arranged to provide an image of the patient's anatomy (e.g. inner wall or any part of the intestine of the patient) and/or an image of all or part of catheter 200 or other portion of system 10, as described in detail herein. Imaging device 70 can comprise an imaging device selected from the group consisting of: endoscope camera; visible light camera; infrared camera; X-ray imager; fluoroscope; CT Scanner; MRI; PET Scanner; ultrasound imaging device; molecular imaging device; and combinations of one or more of these. In some embodiments, a patient image is used to set, confirm and/or adjust one or more system 10 parameters, such as when imaging device 70 comprises a sensor of the present inventive concepts configured to produce a signal.

In some embodiments, system 10 further comprises one or more agents, agent 80 shown. Agent 80 can be delivered by one or more components of system 10, such as by endoscope 50 (via one or more working channels of endoscope 50) and/or by catheter 200 (e.g. via one or more injectate delivery elements 520 or ports 470). Agent 80 can comprise a material selected from the group consisting of: anti-peristaltic agent, such as L-menthol (i.e. oil of peppermint); glucagon; buscopan; hyoscine; somatostatin; a diabetic medication; an analgesic agent; an opioid agent; a chemotherapeutic agent; a hormone; and combinations of one or more of these. In some embodiments, agent 80 comprises cells delivered into the intestine, such as living cells delivered into intestinal mucosa or submucosa via one or more injectate delivery elements 520. In some embodiments, agent 80 comprises one or more agents configured to be delivered into expandable element 530 and to pass through at least a portion of expandable element 530 and into the intestine (e.g. when expandable element 530 comprises at least a portion that is porous). In some embodiments, agent 80 comprises a mucolytic agent configured to remove mucus from a tissue surface.

In some embodiments, system 10 comprises a tissue marker, marker 90, which can comprise a dye or other visualizable media configured to mark tissue (e.g. using a needle-based tool, and/or a visualizable temporary implant used to mark tissue, such as a small, temporary anchor configured to be attached to tissue and removed at the end of the procedure or otherwise passed by the natural digestive process of the patient shortly after procedure completion). Marker 90 can be deposited or deployed in reference to (e.g. to allow an operator to identify) non-target tissue (e.g. a marker positioned proximate the ampulla of Vater to be visualized by an operator to avoid damage to the ampulla of Vater), and/or to identify target tissue (e.g. tissue to be ablated). In some embodiments, marker 90 is deposited or deployed in reference to tissue selected from the group consisting of: gastrointestinal adventitia; duodenal adventitia; the tunica serosa; the tunica muscularis; the outermost partial layer of the submucosa; ampulla of Vater; pancreas; bile duct; pylorus; and combinations of one or more of these.

Shaft 401 can comprise at least six lumens, or at least eight lumens (e.g. as described herebelow in reference to FIGS. 11, 11A-D). In some embodiments, shaft 401 comprises a single shaft comprising the at least six lumens or at least eight lumens. In these embodiments, a first pair of shaft 401 lumens can be in fluid communication with a first tissue capture chamber 510, a second pair of shaft 401 lumens can be in fluid communication with a second tissue capture chamber 510; and at third pair of shaft 401 lumens can be in fluid communication with expandable element 530 (e.g. via openings in shaft 401, ports 430 and 460). The first pair of shaft 401 lumens can comprise a vacuum lumen and a lumen that slidingly receives a first tube attached to a first injectate delivery element 520. The second pair of shaft 401 lumens can comprise a vacuum lumen and a lumen that slidingly receives a second tube attached to a second injectate delivery element 520. The third pair of shaft 401 lumens can comprise a fluid delivery lumen that delivers fluid to expandable element 530 and a fluid removal lumen that removes fluid from expandable element 530 (e.g. via ports 430 and 460, respectively), as described in detail herebelow. In some embodiments, the at least one flexible elongate shaft comprises at least eight lumens, and a fourth pair of lumens are in fluid communication with a third tissue capture chamber 510. In some embodiments, shaft 401 further comprises, as described in detail herebelow, one or more of: a guidewire lumen; a first insufflation lumen; and/or a second insufflation lumen. In some embodiments, shaft 401 comprises multiple shafts, such as two shafts 401 that each include at least a pair of lumens, or three shafts 401 that each include at least a pair of lumens.

In some embodiments, shaft 401 comprises a first lumen for delivering fluid to expandable element 530 (e.g. delivering to element 530 one or more of: inflation fluid 135, ablative fluid 145 and/or neutralizing fluid 150), and a second lumen for removing fluid from expandable element 530 (e.g. removing from element 430 one or more of inflation fluid 135, ablative fluid 145 and/or neutralizing fluid 155). In some embodiments, shaft 401 comprises two, three or more lumens configured to provide and remove fluid from expandable element 530 in a recirculating manner.

Expandable element 530, 540 and/or 550 (singly or collectively expandable element 530) can comprise various materials and dimensions that are configured to optimize the performance of one or more functions, such as submucosal tissue expansion (e.g. duodenal submucosal tissue expansion), mucosal tissue treatment (e.g. duodenal mucosal tissue ablation or other treatment), and/or substance delivery (e.g. delivery of one or more substances into the mucosa, submucosa, and/or other luminal wall location of the duodenum, jejunum, ileum, and/or other GI wall location). In some embodiments, expandable element 530 comprises a diameter (e.g. an expanded diameter of a balloon-based expandable element 530) of at least 5 mm and/or of no more than 45 mm, such as a diameter of at least 18 mm and/or of no more than 32 mm, such as a diameter of at least 23.5 mm and/or no more than 26.5 mm, such as a diameter of approximately 24 mm or 25 mm. In some embodiments, expandable element 530 comprises a balloon with a wall thickness (e.g. thickness of a single wall of the balloon) of at least 0.0001 in and/or of no more than 0.01 in, such as a wall thickness of at least 0.00025 in and/or no more than 0.003 in, such as a wall thickness of at least 0.0005 in and/or no more than 0.001 in, such as a wall thickness of approximately 0.00075 in. In some embodiments, expandable element 530 comprises a balloon with varied wall thickness, such as wall thickness that varies and has a thickness of at least 0.00025 in and/or no more than 0.003 in. For example, expandable element 530 can comprise an increased wall thickness proximate tissue capture cambers 510. In some embodiments, expandable element 530 comprises a material selected the group consisting of: PET; polyimide; nylon, nylon 12; PEEK; a silicone elastomer; polyether block amide; a polyurethane; a thermoplastic elastomer; and combinations thereof. In some embodiments, expandable element 530 (e.g. a balloon-based expandable element 530) comprises a compliance of at least 0.0001% and/or no more than 200%, such as a compliance of at least 0.0001% and/or no more than 15%, such as a compliance between at least 0.0001% and/or no more than 8%. In some embodiments, expandable element 530 comprises one or more materials with a thermal conductivity (W/(m*K)) of at least 0.01 and/or nor more than 10, such as a thermal conductivity of at least 0.1 and/or no more than 0.6, such as a thermal conductivity of approximately 0.29. In some embodiments, expandable element 530 comprises a contact length (e.g. a length of expandable element 530 in contact with duodenal or other luminal wall tissue when inflated or otherwise expanded) of at least 5 mm and/or no more than 500 mm, such as a contact length of at least 10 mm and/or no more than 50 mm, such as a contact length of at least 19 mm and/or no more than 21 mm, such as a contact length of approximately 20 mm. In some embodiments, expandable element 530 (e.g. an inflated balloon-based expandable element 530) comprises a tapered proximal and/or distal end, such as a tapered end with a taper angle (e.g. a proximal and/or distal taper angle as shown in FIG. 11) of at least 5° and/or no more than 120°, such as a taper angle of at least 30° and/or no more than 90°, such as a taper angle of at least 57° and/or no more than 63°, such as a taper angle of approximately 60°. Expandable element 530 can comprise proximal and distal tapers that are similar or dissimilar. In some embodiments, expandable element 530 comprises a balloon which includes a braid on and/or within its wall, such as a metal braid and/or non-metal braid (e.g. a nylon braid).

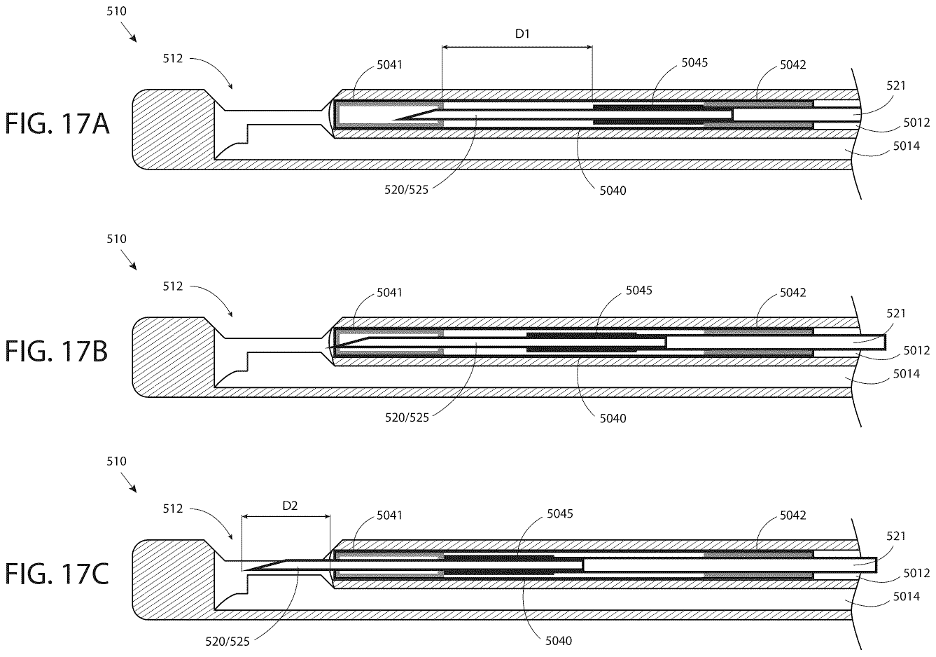

Injectate delivery elements 520 can comprise one or more needles or other fluid delivery elements as described hereabove. Injectate delivery elements 520 can comprise one or more needles or other fluid delivery elements that are configured to deliver fluid or other material to tissue to perform one or more functions, such as submucosal tissue expansion (e.g. duodenal submucosal tissue expansion), mucosal tissue treatment (e.g. duodenal mucosal tissue ablation or other treatment), and/or substance delivery (e.g. delivery of one or more substances into the mucosa, submucosa, and/or other luminal wall location of the duodenum, jejunum, ileum, and/or other GI wall location). In some embodiments, injectate delivery elements 520 comprise elements (e.g. needles) constructed of a material selected from the group consisting of: metal; stainless steel, plastic; PEEK, liquid crystal polymer; and combinations of these. In some embodiments, injectate delivery element 520 comprises one or more needles with an inner diameter of at least 0.0014 in and/or no more than 0.033 in, such as an inner diameter of at least 0.00625 in and/or no more than 0.01325 in, such as an inner diameter of at least 0.0075 in and/or no more than 0.009 in, such as an inner diameter of approximately 0.008 in. In some embodiments, injectate delivery element 520 comprises one or more needles constructed and arranged to have an exposed length (e.g. exposed length D2 defined herebelow in reference to FIG. 17C) of at least 0.125 mm and/or no more than 10 mm, such as an exposed length of at least 1 mm and/or no more than 5 mm, such as an exposed length of at least 2 mm and/or no more than 3 mm, such as an exposed length of approximately 2.5 mm. In some embodiments, injectate delivery element 520 comprises one or more needles with a diameter (e.g. Birmingham gauge) of at least 36gauge and/or no more than 10gauge, such as a gauge of at least 35 and/or no more than 20, such as a gauge of at least 27 and/or no more than 26. In some embodiments, injectate delivery element 520 comprises one or more needles with a bevel angle of at least 1° and/or no more than 90°, such as a bevel angle of at least 5° and/or no more than 45°, such as a bevel angle of at least 9° and/or no more than 11°, such as a bevel angle of approximately 10°.

Console 100 can comprise one or more fluid supplies, as described hereabove, such as to deliver fluid to one or more injectate delivery elements 520. In some embodiments, console 100 is configured (e.g. during a submucosal tissue expansion procedure) to provide fluid to each injectate delivery element 520 at a flow rate of at least 0.1 mL/min and/or no more than 120 mL/min, such as a flow rate of at least 1 mL/min and/or no more than 60 mL/min, such as a flow rate of at least 5 mL/min and/or no more than 20 mL/min, such as a flow rate of approximately 12.5 mL/min. In some embodiments, console 100 is configured (e.g. during a submucosal tissue expansion procedure) to provide, to each injectate delivery element 520, an injection volume (e.g. for delivery at each injection site) of at least 0.1 mL and/or no more than 100 mL, such as an injection volume of at least 1 mL and/or no more than 30 mL, such as an injection volume of at least 8 mL and/or no more than 12 mL, such as an injection volume of at least 9 mL and/or no more than 11 mL, such as an injection volume of approximately 10 mL. In some embodiments, console 100 is configured to provide fluid, to each injectate delivery element 520 (e.g. during a submucosal tissue expansion procedure), at a pressure of at least 1 psi and/or no more than 400 psi, such as at a pressure of at least 20 psi and/or no more than 200 psi, such as at a pressure of at least 90psi and/or no more than 110psi, such as at a pressure of approximately 100psi.

Catheter 200 can comprise multiple fluid-carrying conduits as described hereabove.

For example, multiple conduits 521, also described hereabove, can each attach to a fluid delivery element 520 and travel to the proximal end or at least a proximal portion of catheter 200 (e.g. conduits 521 positioned within shaft 501 and fluidly attached to a port of handle assembly 300). In some embodiments, one or more conduits 521 comprises an inner diameter of at least 0.005 in and/or no more than 0.125 in, such as an inner diameter of at least 0.04 in and/or no more than 0.10 in, such as an inner diameter of at least 0.0177 in and/or no more than 0.0183 in, such as an inner diameter of approximately 0.018. In some embodiments, one or more conduits 521 each comprises a length of at least 12 in and/or no more than 250 in, such as a length of at least 36 in and/or no more than 120 in, such as a length of approximately 78 in.

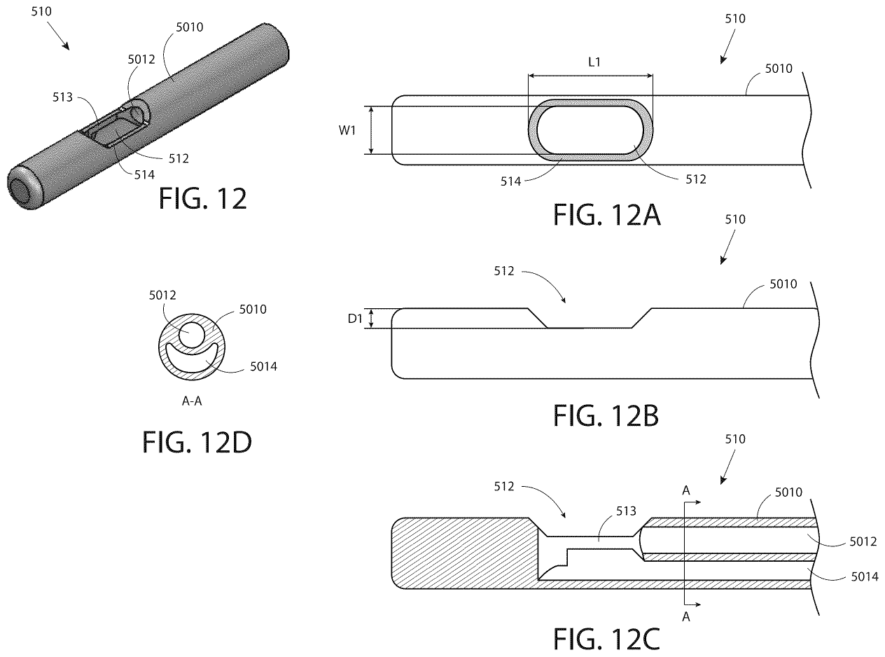

Functional assembly 500 can comprise one, two, three, or more tissue capture chambers 510, such as are described hereabove. Tissue capture chambers 510 can comprise one or more materials selected from the group consisting of: a plastic; a liquid crystal polymer; a metal; stainless steel; a thermally conductive material; and combinations of these. Each tissue capture chamber 510 can be sized and arranged to capture tissue when a vacuum is applied to tissue capture chamber 510. Each tissue capture chamber 510 can be attached (e.g. fixedly attached) to expandable element 530 via an adhesive with a glass transition temperature (Tg) of at least −60° C. and/or no more than 200° C., such as a Tg of at least 60° C. and/or no more than 90° C., such as a Tg of approximately 77° C. Alternatively or additionally, one or more tissue capture chambers 510 can be attached to expandable element 530 via visco elastic tape and/or thermal welding. Each tissue capture chamber 510 can be attached (e.g. fixedly attached) to expandable element 530 via an adhesive configured to support an elongation (e.g. without failure) of at least 1% and/or no more than 500%, such as an elongation of at least 100% and/or no more than 400%, such as an elongation of approximately 300%. Each tissue capture chamber 510 can comprise an outer diameter of at least 0.1 mm and/or no more than 10 mm, such as a diameter of at least 1 mm and/or no more than 5 mm, such as at diameter of at least 2.28 mm and/or no more than 2.30 mm, such as a diameter of approximately 2.29 mm. Each tissue capture chamber can comprise a length of at least 2.5 mm and/or no more than 500 mm, such as a length of at least 10 mm and/or no more than 50 mm, such as a length of at least 17.25 mm and/or no more than 17.75 mm, such as a length of approximately 17.5 mm. Each tissue capture chamber 510 comprises an opening 512. Each opening 512 can comprise a length of at least 1 mm and/or no more than 20 mm, such as a length of at least 2 mm and/or no more than 10 mm, such as a length of at least 3.45 mm and/or no more than 3.65 mm, such as a length of approximately 3.55 mm. Each opening 512 can comprise a width of at least 0.1 mm and/or no more than 10 mm, such as a width of at least 0.5 mm and/or no more than 4 mm, such as a width of at least 1.48 mm and/or no more than 1.68 mm, such as a width of approximately 1.58 mm. Each opening 512 can comprise a depth of at least 0.1 mm and/or no more than 10 mm, such as a depth of at least 1 mm and/or no more than 4 mm, such as a depth of at least 1.9 mm and/or no more than 2.1 mm, such as a depth of approximately 2.0 mm. Each opening 512 can be defined by walls that extend from the outer surface of port 510, such as is described herebelow in reference to FIGS. 12A-C.

Referring now to FIG. 1A, a schematic view of a system for performing a medical procedure in the intestine of a patient is illustrated, consistent with the present inventive concepts. System 10 comprises console 100 and catheter 200. Console 100, catheter 200, and/or other components of system 10 can be of similar construction and arrangement to those described hereabove in reference to FIG. 1. Console 100 of FIG. 1A comprises at least vacuum supply 110, injectate supply 120, ablation fluid supply 140, and neutralizing fluid supply 150, each of which can be included within a single or multiple housings of console 100. Console 100 can include other fluid supplies and assemblies as described herein. Console 100 is fluidly and otherwise operatively attached to catheter 200, such as via an umbilical or other conduit, not shown but such as umbilical 600 described herein. Console 100 comprises one or more pumps, pumping assembly 195, which propels fluids between console 100 and catheter 200, also as described herein.

Catheter 200 comprises a distal portion 208 and a functional assembly 500 which can be positioned on distal portion 208. Functional assembly 500 comprises one or more balloons or other expandable reservoirs, such as reservoir 5301 shown. Console 100 can be configured to transport fluids into and out of reservoir 5301, such as to expand and contract, respectively, reservoir 5301, as described herein.

Catheter 200 further comprises a tissue expansion subsystem 201 configured to expand sub-surface tissue, such as submucosal tissue of the GI tract. Tissue expansion subsystem 201 can comprise conduits within catheter 200 which transport tissue expansion fluids to functional assembly 500 and provide a vacuum to functional assembly 500, each as described herein. Tissue expansion subsystem 201 can comprise at least two tissue capture chambers 510 configured to capture tissue when vacuum is applied via at least two vacuum delivery conduits 511 (e.g. vacuum provided by vacuum supply 110 of console 100). Tissue expansion subsystem 201 can comprise at least two injectate delivery elements 520 (e.g. needles or fluid jets) which can receive the tissue expansion fluid (e.g. injectate 125 provided by injectate supply 120 of console 100) via at least two injectate delivery conduits 521. Injectate delivery elements 520 can be configured to deliver the tissue expansion fluid to tissue captured by tissue capture chambers 510. One or more injectate delivery elements 520 can each comprise a needle configured to penetrate tissue (e.g. via advancement of the needle into chamber 510 when tissue is captured within the chamber 510 via the applied vacuum), after which fluid can be delivered into the tissue. Alternatively or additionally, one or more injectate delivery elements 520 can each comprise a fluid jet configured to deliver fluid through a surface of and into tissue captured within chamber 510.

Catheter 200 further comprises tissue ablation subsystem 202 comprising conduits within catheter 200 which transport ablation fluids and neutralizing fluids to and from functional assembly 500. Tissue ablation subsystem 202 comprises a first conduit, conduit 541, configured to provide fluid to functional assembly 500 (e.g. to reservoir 5301) and a second conduit, conduit 561, configured to remove fluid from functional assembly 500 (e.g. from reservoir 5301). Conduit 541 can be configured to provide to functional assembly 500 ablative fluid (e.g. fluid at an ablative temperature that is provided by ablative fluid supply 140 of console 100), as well as neutralizing fluid (e.g. neutralizing fluid provided by neutralizing fluid supply 150 of console 100 for cooling or warming of tissue prior to and/or after heat ablation or cryogenic ablation, respectively). Conduit 561 can be configured to remove ablative fluid and neutralizing fluid from functional assembly 500. In some embodiments, console 100 is configured to recirculate ablative fluid within functional assembly 500 (e.g. within one or more reservoirs of functional assembly 500), and to also recirculate neutralizing fluid within functional assembly 500 (e.g. within similar or dissimilar reservoirs of functional assembly 500). In some embodiments, console 100 is configured to sequentially recirculate ablative fluid and neutralizing fluid in a single reservoir (e.g. reservoir 5301) of functional assembly 500, such as to heat ablate tissue and subsequently cool tissue, or to pre-cool tissue and subsequently ablate tissue. In some embodiments, console 100 is configured to sequentially recirculate ablative fluid and neutralizing fluid in functional assembly 500 to pre-cool tissue, then ablate tissue, and then cool tissue.

Referring now to FIG. 1B, a flow chart of a method of treating target tissue of a patient is illustrated, consistent with the present inventive concepts. In some embodiments, the method 2200 of FIG. 1B is accomplished using system 10 of FIG. 1 or otherwise as described herein. In Step 2210, a patient is selected for treatment, such as a patient selected to treat and/or diagnose (“treat” herein) a patient disease or disorder selected from the group consisting of: Type 2 diabetes; Type 1 diabetes; “Double Diabetes”; gestational diabetes; hyperglycemia; pre-diabetes; impaired glucose tolerance; insulin resistance; non-alcoholic fatty liver disease (NAFLD); non-alcoholic steatohepatitis (NASH); obesity; obesity-related disorder; polycystic ovarian syndrome (PCOS); hypertriglyceridemia; hypercholesterolemia; psoriasis; GERD; coronary artery disease (e.g. as a secondary prevention); stroke; TIA; cognitive decline; dementia; Alzheimer's Disease; neuropathy; diabetic nephropathy; retinopathy; heart disease; diabetic heart disease; heart failure; diabetic heart failure; and combinations of these. In some embodiments, the patient is selected to treat two or more of the above diseases or disorders, such as a patient selected to treat two or more of diabetes, insulin resistance, NAFLD, NASH, and/or PCOS.

The patient selected can be taking one or more medicines to treat their diabetes. The patient selected can have an HbAlc level between 7.5% and 12.0%, between 7.5% and 10%, or between 7.5% and 9.0%. In some embodiments, the patient selected can have an HbAlc level between 6.0% and 12.0%. Patients with higher HbA1c levels and/or other higher disease burden can receive more aggressive treatments (e.g. more tissue treated and/or higher number of repeated treatments over time) as described herebelow in reference to Step 2250.

Patient selection can be based on the current level of one or more parameters representing one or more various biomarkers or other representative values of physiologic conditions (e.g. as compared to an average among diabetic and/or non-diabetic patients), such as a level of a parameter selected from the group consisting of: body mass index (BMI) level; waist circumference; HbAlc level; fasting glucose; insulin resistance; liver fibrosis; cholesterol or triglyceride level; duration of years exhibiting type 2 diabetes; fasting insulin, fasting C-peptide or C-Peptide stimulation in response to a meal; age; and combinations of these.

Prior to placing one or more devices into the patient (e.g. catheter 200), or at any time thereafter (e.g. during or after the procedure), one or more agents can be introduced into the patient. In some embodiments, one or more agents are introduced into the GI tract directly, such as agent 80 described hereabove in reference to FIG. 1. In some embodiments, agent 80 comprises L-menthol (i.e. oil of peppermint) or other agent configured to provide an anti-peristalsis effect. In these embodiments, a few drops of agent 80 can be placed in an irrigation or other lumen of an inserted device (e.g. endoscope 50). In some embodiments, approximately 8 mL of L-menthol is mixed with approximately 0.2 mL of Tween 80 (polysorbate 80) in approximately 500 mL of distilled water (i.e. to create an approximately 1.6% solution). Approximately 20 mL of this mixture can be sprayed through a working channel of endoscope 50, or more as required to dampen peristalsis. In some embodiments, the solution can vary between approximately 1.6% and 3.2%. Tween and/or sorbitan monostearate can be used as an emulsifier.

One or more agents 80 can be delivered once endoscope 50 or any other agent delivery device of system 10 enters the duodenum. In some embodiments, agent 80 comprises one or more agents that are delivered intravenously, and can include glucagon and/or buscopan.

As described hereabove, in some embodiments, an endoscope is inserted into the patient (e.g. endoscope 50 of FIG. 1). In these embodiments, subsequently inserted devices can be placed through a working channel of endoscope 50 and/or alongside endoscope 50. In some embodiments, endoscope 50 and an attachable sheath are both inserted into the patient, and subsequently inserted devices can be placed through a working channel of endoscope 50, through the attachable sheath, and/or alongside endoscope 50 and the attached sheath. Each patient inserted device can be inserted over a guidewire. In some embodiments, an endoscope stiffening device is used, such as an endoscope stiffening system provided by Zutron Medical of Lenexa, Kansas, USA.

In some embodiments, non-target tissue is identified. Non-target tissue can be identified with a visualization device, such as endoscope 50 and/or imaging unit 70 described hereabove. The non-target tissue can comprise the ampulla of Vater (also known as the papilla), the pancreas, and/or other tissue to which treatment (e.g. ablation) may adversely affect the patient. Marking of the non-target tissue (or tissue proximate the non-target tissue) can be performed, such as with a tattoo, ink or other visualizable substance, such as a visual agent or clip placed in and/or on the mucosa and/or submucosa in or proximate the ampulla of Vater. In some embodiments, one or more markers similar to marker 90 described hereabove in reference to FIG. 1 are deployed in the patient to provide a reference location relative to non-target tissue. Tissue expansion and/or tissue treatment performed in subsequent steps can avoid treating (e.g. avoiding delivering ablative energy to) the non-target tissue identified and potentially marked (e.g. with one or more markers 90).

Next in Step 2210, a treatment catheter, such as catheter 200 of FIG. 1, is inserted through the patient's mouth and advanced through the stomach and into the small intestine. Step 2210 can include selecting a particular model of catheter 200, such as a particular size or other configuration of catheter 200. Catheter 200 can be inserted over guidewire 60, such as are described hereabove in reference to FIG. 1. Guidewire 60 can be advanced such that its distal end is in the jejunum or more distal location. During advancement of catheter 200, guidewire 60 can be held taut in order to prevent catheter 200 from forming a loop in the stomach. As described hereabove, catheter 200 can be inserted through a working channel of endoscope 50 and/or alongside endoscope 50.

Catheter 200 is advanced (e.g. over guidewire 60) such that functional assembly 500 is positioned in the duodenum (or other GI location). One or more tissue capture chambers 510 (e.g. three tissue capture chambers 510 positioned on expandable element 530 of functional assembly 500) can be positioned at a first location in the intestine. The first location can be a most proximal target location to be treated, such as a location in the duodenum at least 1 cm, but not more than 5 cm or 10 cm from the ampulla of Vater. In some embodiments, tissue capture chambers 510 are positioned based on the location of a previously placed marker, such as marker 90 described hereabove. Prior to and/or during insertion of catheter 200, a stiffening wire can be inserted within catheter 200. Endoscope 50 can be positioned adjacent catheter 200, such that the distal ends of each are positioned beyond the ampulla of Vater (e.g. beyond marker 90).

In Step 2220, submucosal tissue expansion is performed, or at least attempted, at the first location (e.g. a first axial segment of the duodenum). Saline and/or other fluid or material (injectate 125) is injected into submucosal tissue. In some embodiments, injectate 125 is delivered (e.g. simultaneously injected) by multiple injectate delivery elements 520 of functional assembly 500, each element 520 positioned in a corresponding tissue capture chamber 510 (e.g. three chambers 510 spaced approximately 120° apart along a circumference). Each injection (by a single injectate delivery element 520) can comprise at least 1 mL, such as at least 2 mL, at least 5 mL or at least 8 mL per each injectate delivery element 520 (e.g. when the cumulative amount of fluid delivered by the multiple injectate delivery element 520 comprises at least 3 mL, such as at least 6 mL, at least 15 mL, or at least 24 mL). Each injection can comprise no more than 20 mL, such as no more than 15 mL, or when each injection comprises approximately 10 mL (e.g. when the cumulative amount of fluid delivered by the multiple injectate delivery element 520 comprises no more than 60 mL, such as no more than 45 mL, or when the cumulative amount comprises approximately 30 mL). In some embodiments, the volume of injectate delivered (e.g. via three circumferentially positioned injectate delivery elements 520) can be configured to achieve an expansion of the submucosal layer to a thickness of at least 250 μm, or approximately 400 μm, in the area surrounding the volume of mucosal tissue to be ablated. Console 100 can be configured to deliver injectate 125 at a flow rate of at least 10 mL/min, such as a flow rate of 50 mL/min, or 100 mL/min. In some embodiments, console 100 is configured to deliver the full volume of injectate for a single injectate delivery element 520 at a single site within a time period of no more than 2 minutes, no more than 1 minute, or no more than 30 seconds.

Volumes injected by the multiple injectate delivery elements 520 can be selected to achieve near full circumferential expansion of submucosal tissue (e.g. without gaps, full 360° expansion). Each submucosal tissue expansion step is configured to create a safety margin of expanded submucosal tissue, as described hereabove, this expanded tissue volume (e.g. a partial or full circumferential tubular volume of the intestine) defining an “expanded tissue periphery”. In some embodiments, functional assembly 500 is constructed and arranged (e.g. the ablative portion is sized) such that a submucosal tissue expansion performed at a single axial location of the small intestine (e.g. via delivery of injectate 125 via two, three or more injectate delivery elements 520, simultaneously or sequentially at the single axial location) creates an expanded tissue periphery that is sufficiently sized to surround an “ablation periphery” that is created during ablation via functional assembly 500 (as described herebelow in reference to Step 2250). This sufficiently sized expanded tissue periphery avoids transmission of significant energy beyond the submucosal layer (e.g. avoids transmission of energy at a level sufficient to ablate the deeper, muscular layers of the GI tract). For example, in cases of full circumferential submucosal tissue expansion, if the axial length of the expanded submucosal tissue achieved by injectate 125 delivery in Step 2220 is greater than the axial length of the tissue to be ablated, the submucosal tissue expanded is sufficient to provide a safety margin for the ablation (e.g. when during ablation functional assembly 500 is relatively centered within the expanded length of tissue).

In some embodiments, the expanded tissue periphery created in a single submucosal tissue expansion step is not sufficiently sized to support the ablation periphery created by functional assembly 500, and an optional Step 2225 is performed (e.g. one or more times), comprising additional submucosal tissue expansion. For example, a second submucosal tissue expansion can be performed at a neighboring (e.g. relatively adjacent and more distal) axial segment of the duodenum, such as by translating (e.g. advancing) catheter 200 to reposition functional assembly 500. Functional assembly 500 can be at least partially collapsed (e.g. ablation fluid 145, neutralizing fluid 155, and/or other fluid is removed from functional assembly 500) prior to translation. Translations of catheter 200 (advancements and/or retractions of functional assembly 500 or other portion of catheter 200) can be performed under visualized guidance, such as when functional elements 499a, 499b and/or 599 described hereabove comprise a radiopaque band or other visualization marker that can be visualized by imaging device 70 (e.g. a fluoroscope). Alternatively or additionally, rotations of catheter 200 (e.g. rotations of functional assembly 500 or other portion of catheter 200) can be performed under similar visualized guidance. In Step 2225, catheter 200 can be translated (e.g. advanced) a pre-determined distance (e.g. a distance of at least 0.3 cm, or at least 0.6 cm), after which delivery of injectate 125 can begin. Delivery of injectate 125 via the injectate delivery elements 520, as described hereabove in reference to Step 2220, creates a second (e.g. contiguous) volume of expanded submucosal tissue that in combination with the first expanded volume of submucosal tissue defines larger expanded tissue periphery than that which is created in a single tissue expansion step. This larger expanded tissue periphery can support larger ablation peripheries (e.g. longer full circumferential lengths of tissue to be ablated), such as may be required by functional assembly 500 in a single ablation. For example, in cases of full circumferential submucosal tissue expansion, if the axial length of the expanded submucosal tissue achieved by injectate 125 delivery in the combined deliveries of Step 2220 and Step 2225 is greater than the axial length of the tissue to be ablated, the submucosal tissue expanded is sufficient to provide a safety margin for the ablation. FIG. 1C illustrates a representative expanded periphery and ablation periphery of two full circumferential expansions followed by a single full circumferential ablation, each performed by catheter 200 via console 100 as described herein. A first and second circumferential submucosal tissue expansion combine to form an expanded tissue periphery with a length as shown. Functional assembly 500 can deliver energy to an ablation periphery that is positioned within the expanded tissue periphery.

Optional Step 2225 can be performed two or more times, resulting in three or more injections of fluid into tissue (e.g. submucosal tissue), with or without an intervening ablation performed via Step 2250. Sequential injections of injectate 125 can be performed at an axial separation distance of between 1 cm and 2 cm apart from a previous injection (e.g. 1 cm to 2 cm distally in the duodenum). In some embodiments, multiple injections are positioned at least 0.5 cm apart along the axis of the duodenum, such as between 1.0 cm and 5.0 cm apart, such as approximately 1.0 cm, 2.0 cm, 3.0 cm, 4.0 cm and/or 5.0 cm apart from one another along the axis of the duodenum. In some embodiments, axial separation of injection sites (i.e. translation distance of catheter 200 between injections) can approximate half the length of expandable element 530 onto which injectate delivery elements 520 are mounted, such as half the length of expandable element 530 of FIG. 1. In some embodiments, a series of 5 -15 sets (e.g. 8-12 sets) of injections (e.g. each set comprising injections from 2, 3 or more injectate delivery elements 520) can be performed (with or without an intervening ablation step) by delivering injectate 125 (e.g. a fluid containing a visualizable dye) to the tissue to be expanded and subsequently translating catheter 200 to a new axial location (e.g. after proper expansion of tissue is confirmed visually as described herebelow in Steps 2230 and 2235, or otherwise). Each advancement and/or retraction of catheter 200 can be made in unison with advancement and/or retraction of an endoscope positioned alongside catheter 200.

As described hereabove, tissue expansion can begin at a location proximate but distal to the ampulla of Vater, such as at a location at least 1 cm distal to but not more than 5 cm or 10 cm from the ampulla of Vater. A series of relatively contiguous, full circumferential submucosal tissue expansions can be performed (e.g. moving distally), for example to a distal location up to the Ligament of Treitz. In alternate embodiments, multiple full circumferential tissue expansions are performed by moving catheter 200 from distal to proximal locations, or in a discontinuous (back and forth) manner.

Volumes of injections and/or axial separation of injection can be chosen to avoid axial gaps between neighboring expanded volumes of tissue (e.g. when an ablation step is to be performed proximate one or both expanded volumes of tissue). After injections, gaps identified circumferentially and/or axially (e.g. via endoscope camera, fluoroscope or ultrasound imaging device), can be filled in as deemed necessary via additional injection (e.g. with or without rotation and/or translation of catheter 200).

In some embodiments, console 100 is configured to reduce the amount of fluid (e.g. liquid such as water or gas such as air or carbon dioxide) in expandable element 530 supporting injectate delivery elements 520 as the injectate 125 is delivered into tissue, such as to prevent excessive force being applied to tissue proximate the expanding tissue (i.e. due to the decreasing lumen of the intestine proximate the expanding tissue in contact with expandable element 530).

Multiple injections (e.g. two, three or more injections from two, three or more equally separated injectate delivery elements 520) can be performed simultaneously or sequentially in a single axial segment of the intestine (e.g. without moving functional assembly 500). A vacuum can be applied (e.g. automatically or otherwise via system 10, such as via a working channel of endoscope 50 and/or via ports 470_{P} or 470_{D} of catheter 200) to the intestinal lumen (e.g. desufflation) prior to delivery of injectate 125, such as to draw tissue toward each injectate delivery element 520 (e.g. into the associated chambers 510). After injectate 125 delivery, the vacuum can be removed and an ablation performed (e.g. in Step 2250 below without additional translation or other movement of functional assembly 500), or catheter 200 can be advanced (or retracted) for a subsequent (additional) tissue expansion.

In Step 2230, an assessment of submucosal tissue expansion is performed (e.g. manually by an operator and/or automatically by system 10). Step 2230 can be performed after Step 2225, as shown in FIG. 1B (e.g. if Step 2225 is performed), and/or directly after Step 2220 (e.g. when a single tissue expansion is sufficient for the subsequent ablation or simply when an assessment is desired directly after a tissue expansion). In some embodiments, assessment of submucosal tissue expansion is performed via a camera view provided by endoscope 50 (e.g. an endoscope with a camera positioned to view the submucosal tissue expansion). Alternatively or additionally, submucosal tissue expansion can be performed using a visualization device of system 10, such as when imaging device 70 described hereabove in reference to FIG. 1 provides one or more images used to perform the assessment. Injectate 125 delivered in Steps 2220 and/or 2225 can include an agent that is directly visualizable by an operator and/or an agent whose location (e.g. a volume of tissue that has been expanded by injectate 125) can be (at least partially) assessed by system 10 (e.g. via an image processing algorithm of console 100 or other component of system 10). For example, injectate 125 can comprise a material selected from the group consisting of: a visible material (such as India Ink, Indigo Carmine, and the like) visualized by an endoscope 50 camera, catheter 200 camera (e.g. when functional element 599 comprises a camera), or other camera; a radiopaque material visualizable by an imaging unit 70 comprising a fluoroscope or other X-ray imaging device; an ultrasonically reflectable material visualizable by an imaging unit 70 comprising an ultrasound imaging device; any visualizable material; and combinations of one or more of these. Visualization of the expanded tissue can be used to determine proper volume of injectate has been delivered as well as sufficient tissue expansion has been achieved, such as sufficient thickness, elimination of gaps, sufficient axial length, and/or sufficient circumferentiality (e.g. full or near-full circumferential nature) of tissue expansion. The pressure of expandable element 530 or the volume of fluid within expandable element 530 can also be monitored to determine if a proper volume of injectate has been delivered to achieve adequate tissue expansion. In particular, the expanded tissue can be analyzed to identify areas of relatively poor expansion which may indicate regions of adherent submucosal tissue (such as scarred and/or fibrotic submucosal tissue not amenable to tissue expansion).

As described above, in some embodiments, assessment of submucosal tissue expansion performed in Step 2230 is performed (at least) using a camera of endoscope 50. In these embodiments, prior to and/or during the assessment of submucosal tissue expansion performed in Step 2230, functional assembly 500 can be at least partially collapsed (e.g. inflation fluid 135, and/or other fluid is removed from functional assembly 500), to provide an increased view of the expanded tissue. Alternatively or additionally, functional assembly 500 is at least partially collapsed to allow advancement of endoscope 50 toward and potentially into the axial segment of intestinal tissue to which the submucosal tissue has been expanded, to provide a closer view of the expanded tissue.

In Step 2235, adequacy of submucosal tissue expansion is determined (e.g. a qualitative assessment performed by a clinician and/or a quantitative assessment performed automatically and/or semi-automatically using system 10). If submucosal tissue expansion is determined to be inadequate, Step 2240 is performed, in which a new (alternative) area for tissue expansion and subsequent ablation is selected, or the procedure is terminated (e.g. after limited or no ablations have been performed). In some embodiments, the method 2200 of FIG. 1B is included in a medical procedure that is performed on a patient after (e.g. at least 24 hours after) a similar procedure has been performed on that same patient (e.g. a similar ablation procedure in the duodenum or other location of the patient's small intestine). The assessment of submucosal expansion performed in Step 2230 can be an important diagnostic test that can confirm that it is safe to perform a repeated, similar procedure (e.g. the procedure of the present inventive concepts). Alternatively, the assessment may enable the identification of patients who may have: an active infection in their duodenum; a history of infection (such as tuberculosis) and/or malignancy that can cause a duodenal injury (e.g. a condition that may make submucosal expansion challenging or even impossible); and combinations of these, such as patients to which no or limited ablations should be performed. For example, there may be significant fibrosis and/or significant scar present at a target location (from a previous procedure or otherwise), which could prevent proper submucosal tissue expansion. In these instances, ablation should not be performed, at least not at that location of the intestine.

If the submucosal tissue expansion is determined to be adequate, Step 2250 is performed in which target tissue is treated (e.g. ablated) by functional assembly 500 of catheter 200. The target tissue can comprise one or more portions of the mucosal layer of the duodenum, jejunum, and/or other GI location proximate (e.g. on top of) the submucosal tissue that has been previously expanded (e.g. in one or more expansion steps 2220 and/or 2225). Treated tissue can further comprise at least an inner layer of neighboring submucosal tissue (e.g. a partial depth of the submucosal tissue layer previously expanded). In some embodiments, the ablation of Step 2250 is performed without repositioning (e.g. without translating) functional assembly 500, such as without repositioning after Step 2220 or without repositioning after Step 2225 (if the optional step is performed), such as to ensure that ablation is performed over an area of expanded submucosal tissue (e.g. over a sufficiently sized expanded tissue periphery as defined herein) that provides a safety margin to avoid adversely effected tissue layers beyond (deeper than) the submucosal layer. One or more circumferential ablations, partial circumferential ablations, and/or other treatments can be performed along a length of the GI tract (e.g. along one or more axial segments of the GI tract), such as along a length of the duodenum at least 1 cm distal to the ampulla of Vater, such as at a location at least 1 cm distal to but within 3 cm, 5 cm or 10 cm of the ampulla of Vater. In some embodiments, all ablations are performed at least 2 cm or at least 3 cm distal to the ampulla of Vater (e.g. tissue within 1 cm, 2 cm or 3 cm of the ampulla of Vater is not ablated). In some embodiments, tissue treatments are only performed at locations that have had submucosal tissue expansion performed and/or confirmed (e.g. visually as described hereabove in reference to Step 2230 and 2235).

In some embodiments, a thermal ablation is provided by sufficiently hot or sufficiently cold fluid introduced into expandable element 530 to ablate tissue. Alternatively or additionally, different forms of energy delivery or other tissue treatments can be performed (e.g. electromagnetic energy, light energy, mechanical energy and/or chemical energy).

Catheter 200 and console 100 can be configured to treat a series of axial segments of GI tract tissue comprising lengths between 1 cm and 5 cm each, such as approximately 2 cm in length each. Catheter 200 and console 100 can be configured to treat a cumulative axial length of GI tract tissue (e.g. an axial length of duodenal mucosa tissue) of less than or equal to 3 cm, 6 cm, 9 cm, 15 cm, or 20 cm. Catheter 200 and console 100 can be configured to treat more than 3 cm of axial length of duodenal mucosa, such as more than 3.4 cm, more than 6 cm, more than 7 cm, more than 8 cm or more than 9 cm (e.g. approximately 9.3cm). In some embodiments, at least 10%, 15%, 25%, 30% and/or 50% of the duodenal mucosa distal to the ampulla of Vater is treated. The axial length and/or overall volume of tissue treated can correspond to a patient parameter, such as the longevity of the disease or other disease parameter as described in detail herebelow (e.g. higher disease burden correlating to larger volumes of tissue treated).

In some embodiments, at least 3 axial segments of duodenal mucosal tissue are treated (e.g. sequentially ablated, such as a sequential treatment including at least one submucosal tissue expansion step performed before each ablation), such as with a treatment element configured to deliver energy to a delivery zone with a length between 0.5 cm and 4.0 cm (e.g. tissue contacting length of expandable element 530 filled with ablative fluid), such as a delivery zone length (e.g. tissue contacting length) between 0.5 cm and 4.0 cm, between 1.5 cm and 3.3 cm, or approximately 2 cm in length. In some embodiments, at least 4 axial segments of duodenal mucosal tissue are treated, such as when at least 6 axial segments of duodenal mucosal tissue are treated. In these embodiments, functional assembly 500 can be configured to deliver energy to a delivery zone with a length between 0.7 cm and 2.0 cm (e.g. tissue contacting length of expandable element 530 filled with ablative fluid). In some embodiments, functional assembly 500 comprises ablative fluid delivered into expandable element 530 (e.g. ablative fluid 145 provided by console 100). Multiple tissue treatments are performed by repositioning functional assembly 500, which can further include contracting expandable element 530 to reposition functional assembly 500. Contact between the target tissue and functional assembly 500 can be accomplished using desufflation techniques to bring the tissue toward expandable element 530 and/or via expansion of expandable element 530. Tissue treatment is performed, such as by filling expandable element 530 with ablative temperature fluid and/or delivering any form of energy to the target tissue. In embodiments where catheter 200 is delivered over a guidewire, the guidewire can be retracted (e.g. at least retracted to a location proximal to the treatment element) prior to any tissue treatments (e.g. prior to any energy deliveries).

Multiple treatments can be performed by advancing or retracting functional assembly 500 and/or catheter 200. In some embodiments, functional assembly 500 is positioned at a distal location and a series of tissue treatments are performed, such as at least 3 tissue treatments performed in which catheter 200 is retracted approximately the length of the tissue contacting portion of functional assembly 500 such as to treat relatively contiguous, non-overlapping, full circumferential axial segments of the duodenum (e.g. where at least one submucosal tissue expansion is performed prior to each ablation or other treatment). Prior to each treatment, an assessment of adequate submucosal tissue expansion can be performed, as described hereabove. Also prior to each tissue treatment, confirmation of being away from (e.g. distal to) any non-target tissue marked and/or otherwise identified can be performed (e.g. by visualizing a previously placed marker 90). In some embodiments, a marker 90 is placed to avoid any damage to the ampulla of Vater. In some embodiments, after three axial segments of duodenal mucosa are treated (e.g. treated distally to proximally), an assessment of the linear distance between the most proximal treatment segment and the ampulla of Vater is performed (e.g. one or more components of system 10 is used to determine the distance). If sufficient length is determined (e.g. the determined distance is above a threshold), additional (more proximal) axial tissue segments can be treated. During translation of catheter 200 over a guidewire, undesired movement of the guidewire is prevented or otherwise reduced by the operator.

In some embodiments, the system of the present inventive concepts (e.g. system 10 of FIG. 1) is configured to allow only one ablation per (pre-determined) time period, such as to prevent two ablations within the time period such as to prevent repetitive ablation in the same or at least similar (e.g. overlapping) portions of the GI tract (e.g. rapid treatment of similar treatment zones).

In some embodiments, the tissue treatment of Step 2250 should be completed within approximately 120 minutes or within approximately 60 minutes of the initiation of tissue expansion performed in Step 2220 and/or step 2225, such as within approximately 45 minutes, 30 minutes and/or 20 minutes. Performance of tissue treatment within this time window prevents an unacceptable amount of injectate 125 dissipation from the expanded submucosal tissue space. In some embodiments, system 10 is configured to prevent a tissue treatment (e.g. ablation) until an adequate submucosal expansion step has been performed and/or confirmed, such as is described in Step 2230. After one or more axial segments of duodenum or other GI segment is ablated in Step 2250, a determination is made in Step 2260 regarding additional axial segments to be treated. In some embodiments, a single axial segment is ablated in Step 2250, after which additional submucosal tissue is expanded (e.g. in one or more of Steps 2220 and/or 2225) and an additional ablation is performed proximate the additionally expanded submucosal tissue. In some embodiments, two axial segments of submucosal tissue are expanded for each single axial segment of mucosal tissue ablated. In some embodiments, a first ablation is performed proximate an area of two submucosal expansions (e.g. directly after the two submucosal expansions are performed), and subsequent ablations are performed after (e.g. directly after) two or less (e.g. one) submucosal expansions are performed (e.g. expansions performed in the area of the subsequent ablations).

The cumulative amount of target tissue treated and/or the number of treatments performed can correlate to (e.g. be proportional to) one or more patient conditions (e.g. more severe correlates to more tissue treated and/or more treatments performed over time). This increased treatment can comprise an increased axial length of tissue treated (e.g. an increased cumulative axial length of duodenum ablated), an increased volume of tissue treated (e.g. an increased volume of duodenal mucosa treated via an increased mucosal surface area receiving ablation energy from functional assembly 500), a deeper depth of treatment, and/or a larger number of treatments performed over time in order to achieve a sustained treatment response. In some embodiments, the tissue treatment is modified to avoid creation of a duodenal stenosis or stricture, such as to limit one or more of: amount of energy delivered; peak energy delivered; duration of energy delivered; length of tissue treated; depth of tissue treated; and combinations of these.

Referring now to FIGS. 2 & 2A, perspective views of an example handle portion of catheter 200 are illustrated, consistent with the present inventive concepts. Handle assembly 300 comprises housing 301 which surrounds various mechanisms and other assemblies of handle assembly 300. In FIG. 2A, portions of housing 301 are removed to illustrate components internal to the handle. In some embodiments, a conduit for carrying one or more fluids it attached to handle assembly 300 (e.g. at its proximal end), such as umbilical assembly 600 also shown. Handle assembly 300 operatively attaches (e.g. at its distal end) to shaft assembly 400.

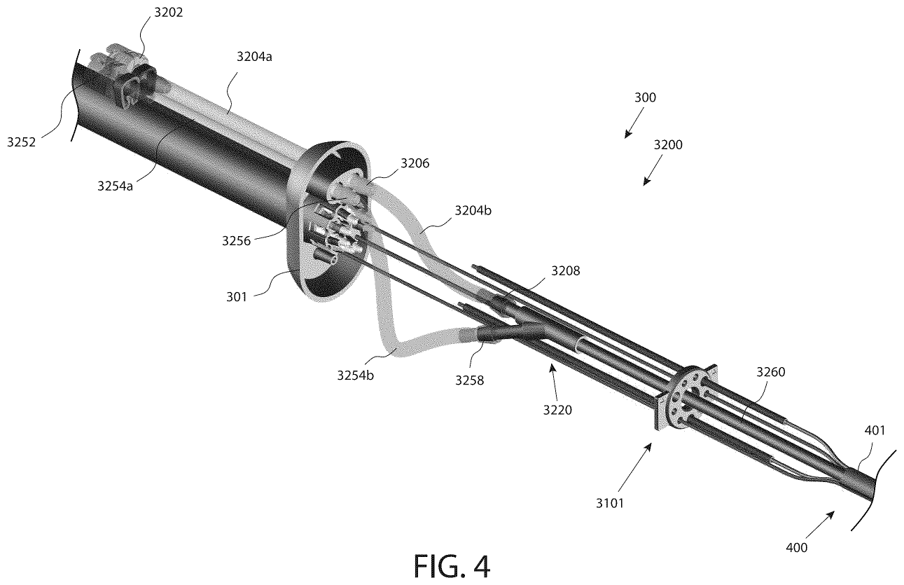

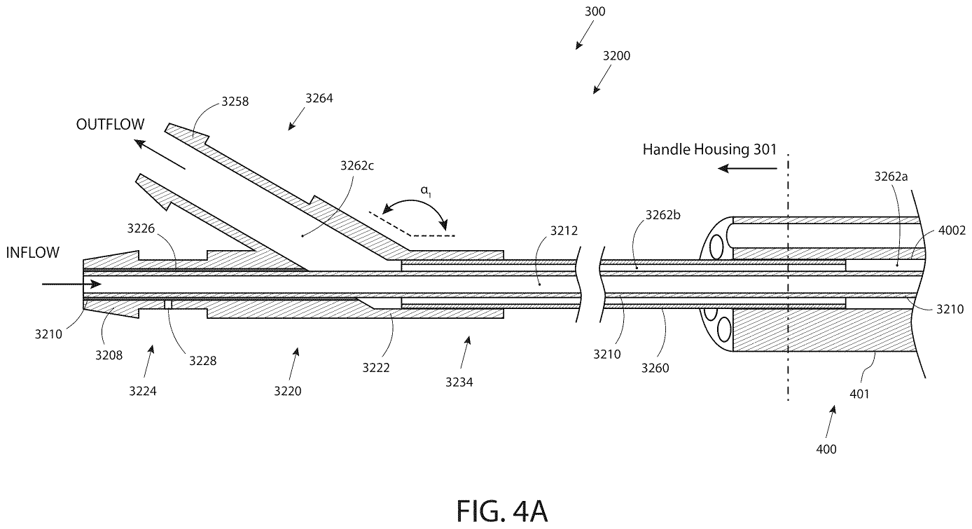

Handle assembly 300 can comprise an assembly, FDE control assembly 3100, configured to manipulate (e.g. advance, retract and/or rotate), operate and/or otherwise control (“control” herein) one or more fluid delivery elements of functional assembly 500. One configuration of FDE control assembly 3100 is described herebelow in reference to FIGS. 3, 3A and 3B. Handle assembly 300 can comprise another assembly, inflation control assembly 3200, configured to control the radial expansion and/or contraction (e.g. inflation and/or deflation) of one or more expandable and/or contractible components of functional assembly 500. One configuration of inflation control assembly 3200 is described herebelow in reference to FIGS. 4 and 4A.

Referring now to FIGS. 3, 3A and 3B, a perspective view, and two side sectional views of one embodiment of FDE control assembly 3100 of handle assembly 300 are illustrated, consistent with the present inventive concepts. In FIG. 3, portions of housing 301 and other components of handle assembly 300 have been removed for illustrative clarity. FDE control assembly 3100 can be configured to control multiple injectate delivery elements 520 (e.g. two, three, four or more injectate delivery elements 520) of functional assembly 500. In the configuration shown in FIG. 3, FDE control assembly 3100 is configured to control three injectate delivery elements 520a-c (e.g. 3 needles, water jets or other fluid delivery elements) of functional assembly 500. In FIGS. 3A-B, a portion of FDE control assembly 3100 is shown, the portion controlling a single injectate delivery element, injectate delivery element 520a. In some embodiments, catheter 200 comprises at least a second injectate delivery element 520b, or at least a third injectate delivery element 520c, and FDE control assembly 3100 comprises at least two or at least three similar portions, respectively.

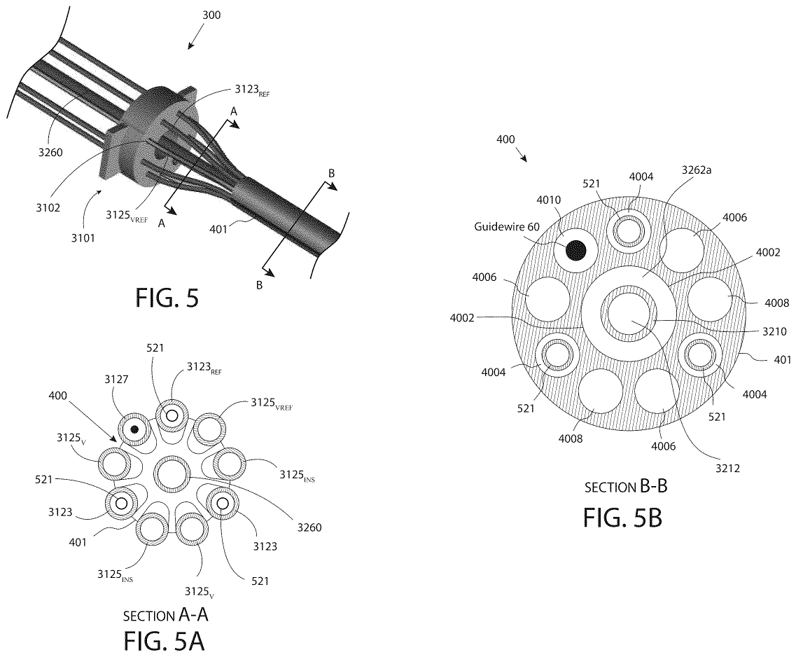

Each injectate delivery element 520 is fluidly connected to the distal end of a conduit, conduit 521. Each conduit 521 is fluidly connected at its proximal end to a conduit arranged in a service loop (e.g. to accommodate the translation of the attached conduit 521), conduit 3124 shown, via connector 3122b. The proximal end of each conduit 3124 fluidly attaches to a conduit of umbilical 600 (or another conduit), via connector 3122a. Each conduit 521 is slidingly positioned within a lumen of shaft 401 of shaft assembly 400, lumen 4004. In some embodiments, shaft 401 comprises a cross sectional profile as described herebelow in reference to FIG. 5B. Each conduit 521 exits the proximal end of each lumen 4004, and is slidingly positioned within a trajectory-determining conduit, guide tube 3123. Each guide tube 3123 comprises a trajectory that allows conduit 521 to traverse from the axis of lumen 4004 to the axis of a linkage within handle assembly 300, linkage 3121 (e.g. the axis of linkage 3121 is a greater distance from central axis Ac than the axis of lumen 4004, as shown and as described in reference to FIG. 5 herebelow). FDE control assembly 3100 comprises multiple aligning components, alignment elements 3101 (3 shown). Alignment elements 3101 are attached to housing 301, such as via tabs 3103 shown, which engage a receiving portion of housing 301 (e.g. slot 303 shown in FIG. 2A). Each alignment element 3101 comprise one or more holes 3102 for positioning, orienting, aligning and/or supporting (“align” or “aligning” herein) various components of FDE control assembly 3100.

Each linkage 3121 surrounds a proximal portion of a conduit 521, and is fixedly attached to that conduit 521 (e.g. via a weld, swage, crimp, adhesive attachment and/or other connection, mechanical connector 3152 as shown). The distal end of each linkage 3121 can be sealed with its surrounded conduit 521 with a potting or other sealing material, seal 3154. Each linkage 3121 slidingly receives the distal portion of a guide tube 3123 such that conduit 521 can pass into guide tube 3123 as shown. Each linkage 3121 can pass through a hole 3102 of one or more alignment elements 3101 (linkage 3121 is shown passing through two alignment elements 3101 in FIGS. 3, 3A & 3B), such as to align linkage 3121. Each linkage 3121 is fixedly attached to a spring, spring 3126 (e.g. via a weld, swage, crimp, adhesive attachment and/or other connection, mechanical connector 3129 as shown). Each spring 3126 (e.g. 3 shown in FIG. 3) of FDE control assembly 3100 fixedly attaches to a sliding element, slide 3110 (e.g. via a weld, swage, crimp, adhesive attachment and/or other connection, mechanical connector 3128 as shown).

Each linkage 3121 (surrounding conduit 521) slidingly passes through a hole, hole 3112 of slide 3110. In some embodiments, a friction-reducing and/or aligning component is included between linkage 3121 and slide 3110, such as bearing 3115 shown. Slide 3110 is fixedly attached to a control, knob 3114 via connector 3113, which passes through an opening, slot 3116, of housing 301. In some embodiments, slide 3110 comprises one or more guides, projections 3111, which can be slidingly received by one or more grooves or other alignment elements of housing 301 configured to slidingly align slide 3110 (e.g. slots 304 shown in FIG. 2).

Translation of knob 3114 translates slide 3110 which in turn applies a corresponding force to springs 3126 which then applies a force to each linkage 3121, causing translation of each conduit 521 (e.g. to advance and/or retract one or more injectate delivery elements 520). In FIG. 3A, knob 3114 is in the retracted position (e.g. injectate delivery elements 520 are in a retracted position). In FIG. 3B, knob 3114 is in the advanced position (e.g. injectate delivery elements 520 are in an advanced position, such as an advanced position in which injectate delivery elements 520 penetrate or at least engage tissue). Inclusion of springs 3126 in the assembly translates conduits 521 (and injectate delivery elements 520) in a force-limiting fashion. For example, slide 3110 can translate a greater distance, distance D1, than the distance D2 translated by each linkage 3121 (e.g. linkage 3121a shown), due to the compensation provided by springs 3126.

Referring now to FIGS. 4 and 4A, perspective and sectional views, respectively, of one embodiment of inflation control assembly 3200 of handle assembly 300 are illustrated, consistent with the present inventive concepts. In FIG. 4, portions of housing 301 and other components of handle assembly 300 have been removed for illustrative clarity. Inflation control assembly 3200, positioned within housing 301, can be constructed and arranged to provide a first pathway to provide a fluid to functional assembly 500, and a second pathway to remove a fluid from functional assembly 500, where the first pathway and second pathway are concentrically oriented. For example, the first pathway can comprise a lumen within an inner conduit that resides within a lumen of an outer conduit, as described herebelow. The second pathway can comprise the space between the outer surface of the inner conduit and the inner surface of the outer conduit, also as described herebelow.

Shaft assembly 400 comprises shaft 401 with a central lumen, lumen 4002. In some embodiments, shaft 401 comprises a cross sectional profile as described herebelow in reference to FIG. 5B. Catheter 200 comprises a fluid transport tube with at least one lumen, conduit 3210 with lumen 3212, that is configured to deliver one or more fluids (e.g. one or more ablative fluids, neutralizing fluids, and/or other fluids) to functional assembly 500. Inflation control assembly 3200 comprises a connecting element, tube 3260 which surrounds a portion of conduit 3210, includes a distal portion that is inserted within lumen 4002, and includes a proximal portion that is inserted into the distal portion of a y-body connector, manifold 3220, all as shown. Manifold 3220 includes housing 3222, defining three hollow arms, arm 3224, arm 3234 (into which tube 3260 is inserted), and arm 3264.

Conduit 3210 can be positioned within a lumen of shaft 401, such as when positioned within lumen 4002 as shown. Conduits 3210 travels proximally from lumen 4002, passing through (e.g. concentrically through) tube 3260, and into arm 3224 of manifold 3220 (via arm 3234) as shown. In some embodiments, a sealing element 3226 (e.g. adhesive, a gasket, an O-ring or the like) is provided between conduit 3210 and arm 3224. In these embodiments, an access port, hole 3228 can be provided, such as to deliver an adhesive to create sealing element 3226 during a manufacturing process.

Catheter 200 comprises a fluid pathway (e.g. lumen), conduit 3262, configured to remove one or more fluids from functional assembly 500. Conduit 3262 includes: a first portion 3262a comprising the space between the outer wall of conduit 3210 and the inner wall of lumen 4002; a second portion 3262b comprising the space between the outer wall of conduit 3210 and the inner wall of tube 3260; a third portion 3262c comprising the space within housing 3222 proximal to the proximal end of tube 3260 (including the space within arm 3264) and excluding the space occupied by conduit 3210; each as shown in FIG. 4A.

In some embodiments, arms 3264 and 3234 are oriented relative to each other at an angle a1. Angle α₁ can be chosen such that the flow pathway geometry of conduit 3262 (in particular the flow pathway between portions 3262b and 3262c) achieves a desired flow dynamic. For example, angle α₁ can be chosen to maintain laminal flow or at least minimize turbulent flow in conduit 3262, such as when angle ai comprises an angle greater than 90°, 120°, 150°or 170°. In some embodiments, arms 3224 and 3234 are collinear as shown. Alternatively, arms 3224 and 3234 are oriented relative to each other at an angle (e.g. not at 180°), such as at an angle similar or dissimilar to angle α₁.

Conduit 3210, at a location proximate the proximal end of arm 3220, is fluidly connected to conduit 3204b via connector 3208. In some embodiments, connector 3208 comprises a barbed portion of arm 3224 of manifold 3220. Conduit 3204b is fluidly connected to conduit 3204a via connector 3206. Conduit 3262 (containing portions 3262a, 3262b and 3262c as shown) is fluidly connected to conduit 3254b via connector 3258. In some embodiments, connector 3258 comprises a barbed portion of arm 3264 of manifold 3220. Conduit 3254b is fluidly connected to conduit 3254a via connector 3256. Conduits 3204a and 3254a can terminate in connectors 3202 and 3252, respectively. Connectors 3202 and 3252 can be configured to fluidly connect to one or more connectors of console 100 and/or umbilical 600, such as to fluidly connect conduits 3210 and 3262 to a source of fluid (e.g. ablative and/or neutralizing fluid) and a fluid removal source, respectively.

Referring now to FIGS. 5, 5A and 5B, a perspective view and two magnified sectional views of an interface between handle assembly 300 and shaft assembly 400 are illustrated, consistent with the present inventive concepts. Housing 301 of handle assembly 300 has been removed for illustrative clarity. FIG. 5A is a sectional view of section A-A of FIG. 5. FIG. 5B is a sectional view of section B-B of FIG. 5. Shaft 401 of FIGS. 5, 5A and 5B comprises a multi-lumen extrusion, shaft 401, comprising a fluid delivery conduit, lumen 4002 (which can be positioned centrally in shaft 401 as shown), and a circumferential array of nine lumens, lumens 4004, 4006, 4008, and 4010 as shown. Positioned within central lumen 4002 is a fluid transport tube, conduit 3210, which includes lumen 3212, each as described hereabove in reference to FIG. 4A. In some embodiments, lumen 3212 provides fluid to functional assembly 500 (e.g. provides all, one or two of: inflation fluid 135 via inflation fluid supply 130, ablative fluid 145 via ablative fluid supply 140, and/or neutralizing fluid 155 via neutralizing fluid supply 150 to expandable element 530). In these embodiments, the space between the outer wall of conduit 3210 and the inner wall of lumen 4002 defines a lumen, conduit 3262a, that transports (removes) fluid from functional assembly 500 (e.g. removes all, one or two of: inflation fluid 135, ablative fluid 145, and/or neutralizing fluid 155 from expandable element 530). Alternatively, conduit 3262a can provide fluids to functional assembly 500 and lumen 3212 can remove fluids from functional assembly 500. In some embodiments, shaft 401 is of similar construction and arrangement to shaft 401 described hereabove in reference to FIG. 1.

Handle assembly 300 includes multiple tubes, guide tubes 3123 (3 shown), conduits 3125_{V} (3 shown), conduit 3125_{INS} (2 shown), and guide tube 3127, each of which are aligned with alignment element 3101. Alignment element 3101 comprises holes 3102, each hole 3102 surrounding the above tubes. One of the guide tubes 3123 is marked as 3123_{REF} and one of the conduits 3125_{V} is marked as 3125_{VREF} to provide information relative to the relative orientation between FIGS. 5 and 5A.

Each of the guide tubes 3123 (3 shown), conduits 3125_{V} (3 shown), conduits 3125_{INS} (2 shown), and guide tube 3127 transition from a first circumferential arrangement defined by holes 3102 of alignment element 3101, to a second, smaller diameter circumferential arrangement defined by the lumens 4002 (1 shown), 4004 (3 shown), 4006 (3 shown), 4008 (2 shown), and 4010 (1 shown), respectively, of shaft 401.

Each guide tube 3123 can surround an inner tube, conduit 521, such as is described hereabove in reference to FIGS. 3, 3A and 3B. Each guide tube 3123 can comprise the same geometry, creating the same path length during translation of each conduit 521, such as to cause equal translations of each distally attached injectate delivery element 520 caused by translation of a single knob 3114 operatively connected to the multiple conduits 521 via the various components of FDE control assembly 3100, also as described hereabove in reference to FIGS. 3, 3a and 3B. Each guide tube 3123 terminates within a corresponding lumen 4004, such as at a location proximal to section B-B, such as a termination less than 1.25″, 1″, or 0.13″ from the proximal end of shaft 401. The distal end or a distal portion of each guide tube 3123 can create a smooth transition with the wall of each lumen 4004, such as a smooth transition including an adhesive or press-fit. In some embodiments, lumens 4004 are spaced relatively equidistantly about a circumference of shaft 401, such as when lumens 4004 comprises three lumens separated by approximately 120° (or alternatively two lumens 4004 separated by 180° or four lumens 4004 separated by 90°). Equidistant spacing of these conduit 521 carrying lumens 4004 can provide an advantage of minimizing path length differences that would affect translation (e.g. cause an undesired effect that could occur during simultaneous translation) of the multiple conduits 521 as described herein.

Each conduit 3125ᵥ connects a vacuum-carrying conduit provided by console 100 (e.g. vacuum supply 110) and/or umbilical 600 to functional assembly 500 (e.g. to tissue capture chamber 510), via lumen 4006. For example, each conduit 3125ᵥ can be fluidly attached on its proximal end to a conduit of umbilical 600 via a connector of handle 300. Each conduit 3125_{V} can travel through handle assembly 300 (e.g. as aligned by one or more alignment elements 3101) and transition (as described above) to align with a corresponding lumen 4006. Each conduit 3125ᵥ can comprise the same geometry, such as a geometry similar to guide tubes 3123, as shown. Each conduit 3125ᵥ terminates within its corresponding lumen 4006, such as at a location proximal to section B-B, such as a termination less than 1.25″, 1″, or 0.13″ from the proximal end of shaft 401. The distal end or a distal portion of each conduit 3125ᵥ can create a seal with the wall of each lumen 4006, such as seal including an adhesive or press-fit.

Each conduit 3125_{INS} connects to a source for insufflation and/or desufflation provided by console 100 (e.g. insufflation supply 170) and/or umbilical 600 to a distal portion of catheter 200 (e.g. to ports 470_{P} and 470_{D} described herein), via lumen 4008. For example, each conduit 3125_{INS} can be fluidly attached on its proximal end to a conduit of umbilical 600 via a connector of handle 300. Each conduit 3125_{INS} can travel through handle assembly 300 (e.g. as aligned by one or more alignment elements 3101) and transition (as described above) to align with a corresponding lumen 4008. Each conduit 3125_{INS} can comprise the same geometry, such as a geometry similar to guide tubes 3123, as shown. Each conduit 3125_{INS} terminates within its corresponding lumen 4008, such as at a location proximal to section B-B, such as a termination less than 1.25″, 1″, or 0.13″ from the proximal end of shaft 401. The distal end or a distal portion of each conduit 3125_{INS} can create a seal with the wall of each lumen 4008, such as seal including an adhesive or press-fit.

In some embodiments, one or more conduits 3125_{INS} connects to a source of a functional fluid provided by console 100 (e.g. functional fluid 185 provided by functional fluid supply 180) and/or umbilical 600 to a distal portion of catheter 200 (e.g. to ports 470_{P} and 470_{D} and/or functional assembly 500 described herein), via lumen 4008. For example, each conduit 3125_{INS} can be fluidly attached on its proximal end to a conduit of umbilical 600 via a connector of handle 300.

Guide tube 3127 provides a pathway for insertion of a guidewire, such as guidewire 60 described hereabove in reference to FIG. 1, such as to allow over-the-wire delivery and removal of catheter 200. Guide tube 3127 provides a pathway from a proximal portion of catheter 200 (e.g. a location on or proximal to handle assembly 300) to a location on a distal portion of catheter 200 (e.g. a location distal to functional assembly 500, via lumen 4010. Guide tube 3127 can travel through handle assembly 300 (e.g. as aligned by one or more alignment elements 3101) and transition (as described above) to align with lumen 4010. Guide Tube 3127 can comprise a geometry similar to guide tubes 3123, as shown. Guide Tube 3127 terminates within its corresponding lumen 4010, such as at a location proximal to section B-B, such as a termination less than 1.25″, 1″, or 0.13″ from the proximal end of shaft 401. The distal end or a distal portion of guide tube 3127 can create a smooth transition with the wall of lumen 4010, such as transition including an adhesive or press-fit.

Shaft 401 can comprise a central lumen, surrounded by multiple satellite lumens, nine satellite lumens shown. Satellite lumens (lumens 4004, 4006, 4008, and 4010 shown) can comprise similar or dissimilar geometries, and can be spaced evenly or non-uniformly about central lumen 4002. The satellite lumens can comprise a diameter greater than 0.020″, such as a diameter of approximately 0.023″, 0.028″, 0.040″, and/or 0.050″. In some embodiments, one or more satellite lumens and/or central lumen 4002 comprise non-circular geometries (e.g. D-shaped, or crescent shaped geometries). In some embodiments, satellite lumens and central lumen 4002 are oriented in an asymmetric geometry (e.g. the central lumen 4002 is not in the center of shaft 401 and/or the satellite lumens do not concentrically surround central lumen 4002). Shaft 401 can comprise an outside diameter of approximately 0.256″.

In some embodiments, two or more lumens of shaft 401 (e.g. as shown in FIG. 5B) are provided as a single lumen, such as when a single lumen provides vacuum to two or more tissue capture chambers 510.

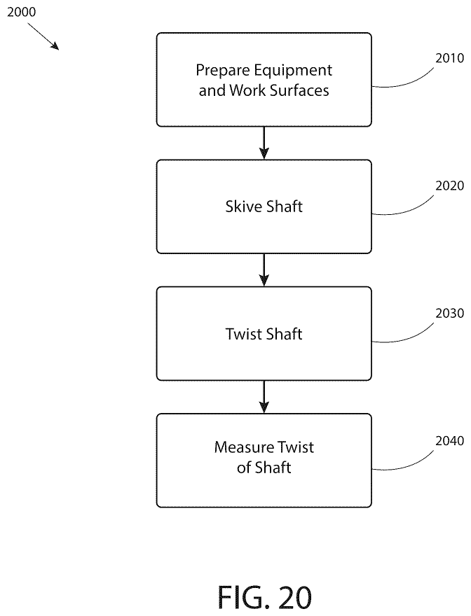

Referring now to FIG. 6, a perspective view of an embodiment of shaft assembly 400 is illustrated, consistent with the present inventive concepts. Shaft 401 of FIG. 6 comprises a single shaft including multiple satellite lumens (e.g. lumens 4004 or other satellite lumens described hereabove in reference to FIG. 5B) around a central lumen 4002. Specifically, the multiple satellite lumens 4004 can be configured to slidingly receive one or more conduits, such as conduit 521 described herein. It can be desirable to equalize the path length from the distal end of shaft 401 to the proximal end of shaft 401, of each lumen 4004, while shaft 401 transverses a tortuous path, such as a path through the duodenum or other portion of the gastrointestinal tract of a patient. Shaft 401 can comprise a twisted geometry along its length, such that each satellite lumen travels in a spiral pattern around the central axis of shaft 401. In some embodiments, shaft 401 comprises a counterclockwise twist, as shown in FIG. 6, such as to minimize path length differences encountered in the GI tract (e.g. a twist opposite to the inherent clockwise path encountered when positioned through the stomach and into the small intestine). In some embodiments, the outer surface of shaft 401 can comprise an indicator, marker 402, such as an elongate stripe along the shaft 401 that is aligned with a single satellite lumen. One or more markers 402 can provide a visual indicator of the twist in shaft 401. A twist can be created in shaft 401 using a tool, such as tool 30 described herebelow in reference to FIGS. 19 and 20. One or more markers 402 can provide a radial indicator of an internal lumen of shaft 401, such as lumens 4004, 4006, 4008, and/or 4010. In some embodiments, one or more markers 402 are used to create a skive into shaft 401 at a proper radial location of shaft 401 (e.g. to provide an opening to a particular lumen of shaft 401), such as is described herebelow in reference to FIG. 20.

In some embodiments, shaft 401 comprises a twist with a varying pitch along its length. For example, shaft 401 can comprise a proximal portion 401a that comprises a first length and a first pitch, and a distal portion 401b that comprises a second length and a second pitch, wherein the second length is different than the first length and/or the second pitch is different than the first pitch. Note that proximal portion 401a and distal portion 401b are not necessarily shown to scale in FIG. 6. In some embodiments, the second pitch is lower than the first pitch (i.e. distal portion 401b comprises more twist per unit length than proximal portion 401a). In some embodiments, proximal portion 401a comprises a single twist (360°) and is approximately three times the length of distal portion 401b which comprises a single twist (360°). Either or both twists can comprise a counterclockwise twist (as shown in FIG. 6), which can be configured to minimize pathway length differences of tubular components within shaft 401 as described hereabove.

In some embodiments, twist imparted on shaft 401 is performed in a heat-setting process in which shaft 401 is maintained in a fixture in a twisted state while heat is applied, such as is described in FIG. 19 herebelow. Additionally or alternatively, the twist imparted on shaft 401 can be imparted during an extrusion process (e.g. as shaft 401 is extruded, the extrusion is twisted at a prescribed rate) to produce a shaft with a “natural” twist. Alternatively, using a multi-tube construction (e.g. instead of a multi-lumen extrusion), satellite “tubes” can be twisted about a central tube (comprising central lumen 4002), and the twisted satellite tubes can be laminated (reflowed) to the central tube in the twisted configuration.

Shaft 401 can comprise a clockwise and/or counterclockwise twist. In some embodiments, shaft 401 comprises a counterclockwise twist (as shown in FIG. 6) configured to minimize pathway length difference of tubular components within shaft 401 as described hereabove.

Referring now to FIG. 7A-B, the distal portion of an embodiment of catheter 200 including functional assembly 500 is illustrated, consistent with the present inventive concepts.

Catheter 200 includes shaft assembly 400 and functional assembly 500, and other components of similar construction and arrangement to those described herein. Shaft assembly 400 comprises a multi-lumen shaft, shaft 401 and a distal tip, tip 410. Shaft assembly 400 can further comprise one or more ports configured to provide insufflation and/or desufflation (“insufflation” herein), such as ports 470_{P} and 470_{D} shown. Ports 470_{P} and 470_{D} can be fluidly attached to one or two lumens of shaft 401, such as is described hereabove in reference to lumens 4008 of FIG. 5B. Ports 470_{P} and 470_{D} can each comprise a diameter between 0.028″ to 0.040″. Shaft assembly 400 can further comprise one or more ports configured to allow a guidewire, such a guidewire 60, to exit shaft 401, such as port 490. Port 490 can be operably attached to a lumen of shaft 401, such as is described hereabove in reference to lumen 4010 of FIGS. 5A-B.

Catheter 200 further includes manifold 700d, including housing 5002, which provides fluid connections between various lumens and other conduits within shaft 401 (proximal to manifold 700d) to various lumens and other conduits that provide and/or remove fluid from functional assembly 500. Functional assembly 500 can comprise a radially expandable and contractible element, expandable element 530 (e.g. a balloon as described herein). Positioned on expandable element 530 are two, three, four or more tissue capture chambers 510 (e.g. three chambers 510a-c shown in FIGS. 7A-B). Chambers 510a-c are each fluidly attached to a multi-lumen shaft, conduits 5010a-c respectively. In some embodiments, conduits 5010a-c each comprise at least two lumens (e.g. a lumen for a tube fluidly connected to an injectate delivery conduit 520 and a lumen for providing a vacuum to a tissue capture chamber 510). Conduits 5010a-c are each fluidly attached to manifold 700d, as described herebelow. A translatable needle or other fluid delivery element, injectate delivery element 520a-c, can be positioned in each respective chamber 510a-c.

Manifold 700d can be constructed and arranged to fluidly combine one or more of lumens of shaft 401. Alternatively or additionally, manifold 700d can be constructed and arranged to split (divide) one or more of lumens of conduit 401 into multiple lumens. In some embodiments, manifold 700d includes one or more valves (e.g. one or more one-way valves) configured to control flow of fluid in a conduit. In some embodiments, manifold 700d includes one or more sensors (e.g. temperature and/or pressure sensors) configured to provide a signal related to a parameter (e.g. temperature and/or pressure) of fluid within a conduit.

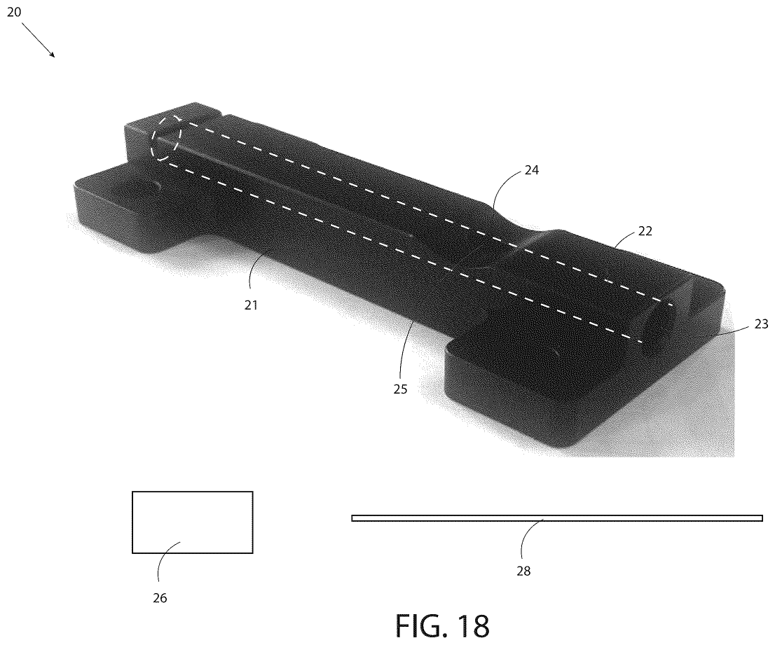

Referring additionally to FIGS. 8A-D, sectional views of shaft assembly 400, manifold assembly 700d and conduit 5010 of FIGS. 7A-B are illustrated, consistent with the present inventive concepts. FIG. 8A illustrates section A-A of FIG. 7B. FIG. 8B illustrates section A′-A′ of FIG. 8A. FIG. 8C illustrates section B-B of FIG. 7B. FIG. 8D illustrates section C-C of FIG. 7B. As shown in FIGS. 8A-B, a skive in shaft 401, skive 5004a, provides access to lumens 4004a and 4006a of shaft 401. Additional skives 5004b and 5004c can be included to provide access to lumens 4004b-c and 4006b-c, respectively, such as when functional assembly 500 comprises multiple tissue capture chambers 510 and/or multiple injectate delivery elements 520, such as three tissue capture chambers 510a-c and/or injectate delivery elements 520a-c. Skives into shaft 401 can be created using a tool, such as tool 20 described herebelow in reference to FIGS. 18 and/or 20. Manifold 700d includes one or more conduits, such a three tubes 5022a-c and three tubes 5024a-c. Tubes 5022a-c operably attach to lumens 4004a-c, respectively, such that conduits 521a-c can translate between lumens 4004a-c and tubes 5022a-c. The proximal portions of tubes 5022a-c are positioned within lumens 4004a-c via skives 5004a-c. In some embodiments, seals 5006a-c (e.g. an adhesive or potting material) are provided between tubes 5022a-c and lumens 4004a-c, respectively. Tubes 5024a-c fluidly attach to lumens 4006a-c, respectively, such that a source of vacuum can be provided to tubes 5024a-c by lumens 4006a-c. The proximal portions of tubes 5024a-c are positioned within lumens 4006a-c, also via skives 5004a-c. In some embodiments, seals 5006a-c (e.g. an adhesive or potting material) are provided between tubes 5024a-c and lumens 4006a-c, respectively.

As shown in FIG. 8C, each of tubes 5022 and tubes 5024 transition, in pairs, from a first orientation to a second orientation. In the first orientation, where each pair of tubes 5022 and 5024 are within lumens 4004 and 4006, each tube (of the pair) is at the same distance from a center axis of shaft 401. In the second orientation, where each pair of tubes 5022 and 5024 are distal to skive 5004, the pair is in a radially stacked arrangement, such as when each tube 5022 is on top of (further away from the center axis or shaft 401) a paired tube 5024. Each tube 5022 operably attaches to a lumen 5012 (e.g. a relatively round lumen) of a conduit 5010, such that conduit 521 translates within tube 5022 and lumen 5014. Each tube 5024 fluidly attaches to lumen 5014 (e.g. a crescent shaped lumen as shown in FIG. 8D) of conduit 5010. Each tube 5024 (e.g. a relatively round tube) can be sealed within a corresponding lumen 5014 (e.g. crescent shaped lumen) with a seal 5016 (e.g. adhesive or potting material).

In the stacked arrangement in which tube 5022 (and connected lumen 5012) is on top of a paired tube 5024 (and connected lumen 5014), a injectate delivery element 520 on the distal end of a conduit 521 within tube 5022 and lumen 5012 is correspondingly positioned above a source of vacuum provided by lumen 5014 via tube 5024 (as shown in FIG. 8D). This orientation provides better engagement of tissue within each tissue capture chamber 510 relative to each injectate delivery element 520 (such as when injectate delivery element 520 comprises a needle or water jet that penetrates or at least engages tissue prior to delivery of injectate 125 into the tissue).

In some embodiments, two or more lumens of shaft 401 of FIGS. 8A-D are provided as a single lumen, such as when a single lumen provides vacuum to two or more tissue capture chambers 510.

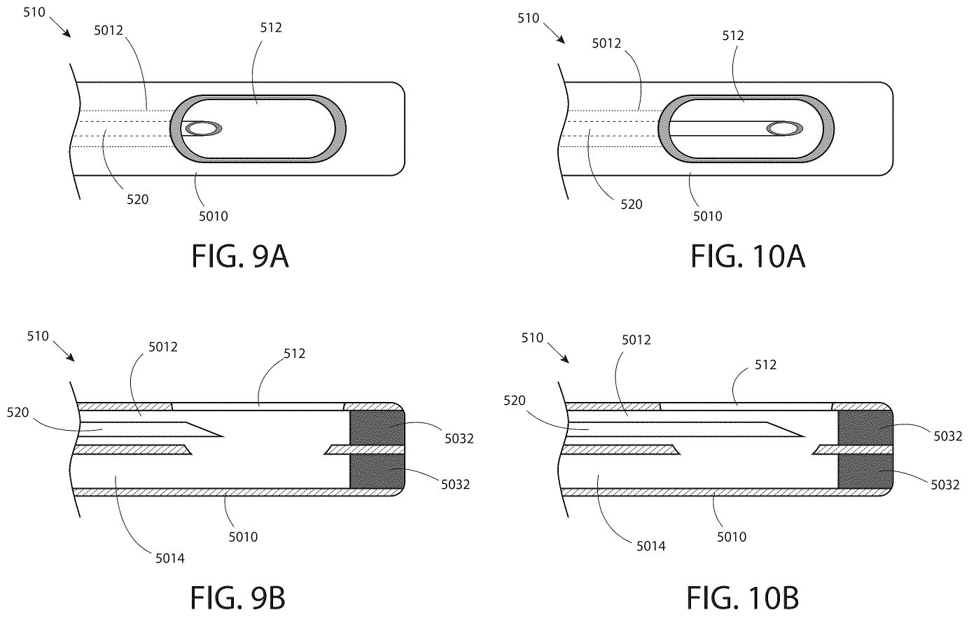

Referring now to FIGS. 9A-B and 10A-B, top and sectional views of an embodiment of a tissue capture chamber are illustrated, consistent with the present inventive concepts. In FIGS. 9A-B, top and side sectional views are shown, respectively, where an injectate delivery element 520 comprising a needle is in a retracted position. In FIGS. 10A-B, top and side sectional views are shown, respectively, where the injectate delivery element 520 has been advanced (e.g. advanced into tissue drawn into tissue capture chamber 510 as described herein, tissue not shown). Catheter 200 can comprise multiple tissue capture chambers 510, such as two chambers 510 separated by 180°, three chambers 510 separated by 120° (as shown in FIGS. 7A-B), four chambers 510 separated by 90°, or more than four chambers 510. Tissue capture chamber 510 can comprise a cylindrical structure with an opening 512 positioned on the top surface of tissue capture chamber 510 (e.g. the top surface being opposite a bottom surface that is oriented toward shaft 401 and attached to expandable element 530 of functional assembly 500, as shown in FIGS. 7A-B). Tissue capture chamber 510 can comprise the distal portion of conduit 5010, as shown, or it can comprise a separate cylindrical tube operably attached to the distal end of conduit 5010 (e.g. a cylindrical tube with a similar cross sectional profile to conduit 5010). Tissue capture chamber 510 comprises a sealed distal end, such as when a sealing element 5032 (e.g. adhesive, potting material, or a plug) is positioned at the distal end of conduit 5010 (e.g. at the distal end of lumens 5012 and 5014).

In operation, a vacuum is applied to lumen 5014 (e.g. a vacuum provided by vacuum supply 110 which is fluidly connected to conduit 3125_{V}, which is fluidly connected to lumen 4006, which is fluidly connected to tube 5024, which is fluidly connected to lumen 5014). When functional assembly 500 is positioned within a GI lumen (e.g. the duodenum), application of the vacuum to lumen 5014 causes tissue to be drawn into tissue capture chamber 510. The capture of tissue (e.g. engagement with tissue) by chamber 510 can be used to maintain contact between functional assembly 500 (e.g. contact with a balloon or other expandable element 530 of functional assembly 500) and tissue, such as during an ablation or other tissue treatment step.

Alternatively or additionally, the capture of tissue by chamber 510 can be used to deliver fluid to tissue, via injectate delivery element 520. In some embodiments, fluid is delivered into tissue captured within tissue capture chamber 510 (e.g. via a water-jet based injectate delivery element 520), when injectate delivery element 520 is in the position shown in FIGS. 9A-B. In other embodiments, injectate delivery element 520 is advanced to the position shown in FIGS. 10A-B (e.g. an advancement caused by translation of knob 3114 of handle assembly 300 as described herein), after which fluid is delivered into tissue captured within tissue capture chamber 510. In some embodiments, chamber 510 is constructed and arranged as described herebelow in reference to any of FIGS. 12-17.

Referring now to FIG. 11, a side sectional view of an embodiment of the distal portion of catheter 200 is illustrated, consistent with the present inventive concepts. In FIGS. 11A-D, sectional views of the distal portion of catheter 200 is illustrated, where FIG. 11A is a sectional view at section D-D, FIG. 11B is a sectional view at section E-E, FIG. 11C is a sectional view at section F-F, and FIG. 11D is a sectional view at section G-G. Catheter 200 comprises a tapered distal tip, tip 410 which includes a conduit, lumen 412, for passage of a guidewire, such as guidewire 60 shown, which is received from lumen 4010 of shaft 401. Tip 410 can comprise a material such as polyether block amide, and a cone-like (e.g. a hollow cone) construction, a double cone (e.g. an inner and an outer cone) construction, or a solid (e.g. not hollow) construction. Lumen 412 can be arranged (e.g. curved) such that an inserted guidewire 60 traverses from a first location (axis of lumen 4010) that is radially offset from central axis Ac of catheter 200, to a location at central axis Ac (as shown), or at least to a location with a smaller radial offset than the axis of lumen 4010. In some embodiments, tip 410 includes a tube comprising lumen 412 (e.g. a tube residing between an inner and outer cone of tip 410), such as a polyimide tube with an approximately . 040″ outer diameter and an approximately 0.038″ inner diameter. Alternatively, tip 410 comprises a solid (e.g. not hollow) construction, and lumen 412 comprises a lumen through the solid material of tip 410, with an inner diameter of approximately 0.038″.

At locations at and distal to Section D-D (“distal to Section D-D” herein), lumens 4004 and 4006 are no longer used (e.g. to transport fluid or other material). In some embodiments, one or more of the distal segments (segments distal to section D-D) of lumens 4004 and 4006 are filled, such as with a sealing element 415 (e.g. an adhesive, potting material, plug or filament), as shown in FIGS. 11A-D.

At location D-D, an opening, port 470_{P} is positioned between lumen 4008_{P} and the outer surface of shaft 401. Port 470_{P} can be configured as a port for insufflation (e.g. providing fluids for insufflation and/or removing fluids for desufflation), at a luminal location (e.g. a GI luminal location) proximal to functional assembly 500. Distal to location D-D, lumen 4008_{P} is also filled with a sealing element 415, as shown in FIGS. 11B-D, such as to direct (e.g. limit) the insufflation source of lumen 4008_{P} to port 470_{P}.

At location E-E, one or more openings, fluid removal ports 460, (3 shown) are positioned between conduit 3262 and the outer surface of shaft 401, fluidly connecting conduit 3262 to the interior of expandable element 530. Ports 460 are created (e.g. punched or drilled) from the outer surface of shaft 401, thru the wall of shaft 401, and thru at least one the sealing elements 415 (two shown per port in FIG. 11B). Fluid removal ports 460 are fluidly connected to conduit 3262, such that fluid can be removed from expandable element 530 via ports 460. Distal to location E-E, a sealing element 5026 is positioned within conduit 3262, between the outer surface of conduit 3210 and the inner surface of central lumen 4002, as shown in FIGS. 11 and 11C-D. Seal 5026 is configured to seal the distal end of conduit 3262 distal to ports 460, and proximal to ports 430, described herebelow.

At location F-F, one or more openings, inflation ports 430, (3 shown) are positioned between the outer surface of shaft 401, and a space, chamber 3212′, created between seal 5026 and a distal seal 5028 shown in FIG. 11, configured to seal the distal end of lumen 4002. Chamber 3212′ is fluidly connected to lumen 3212 of conduit 3210. Ports 430 are created (e.g. punched or drilled) from the outer surface of shaft 401, thru the wall of the shaft, and thru at least one the sealing elements 415 (two shown per port in FIG. 11C). Conduit 3210 terminates within and is fluidly attached to chamber 3212′, which is fluidly attached to the interior of expandable element 530 via ports 430, such that fluid can be inserted into expandable element 530 from console 100.

At location G-G, an opening, port 470_{D} is positioned between lumen 4008_{D} and the outer surface of shaft 401. Port 470_{D} can be configured as a port for insufflation (e.g. providing fluids for insufflation and/or removing fluids for desufflation), at a luminal location (e.g. a GI luminal location) distal to functional assembly 500. Distal to location G-G, lumen 4008_{D} is also filled with a sealing element 415, not shown, such as to direct (e.g. limit) the insufflation source of lumen 4008_{D} to port 470_{D}.

Expandable element 530 (e.g. a balloon when inflated) can comprise a tapered distal end (angle ap as shown), and/or a tapered proximal end (angle ap also as shown). Taper angles α_{D} and α_{P} can comprise similar or dissimilar angles. Expandable element 530 can comprise a distal taper angle ap and/or proximal taper angle ap as described hereabove in reference to FIG. 1.

Referring now to FIGS. 12 and 12A-D, perspective, top, side, side sectional, and sectional views of an embodiment of a tissue capture chamber are illustrated, respectively, consistent with the present inventive concepts. FIG. 12D illustrates a sectional view along section A-A of FIG. 12C. Tissue capture chamber 510 can be of similar construction and arrangement as described hereabove in reference to FIGS. 9A-B and 10A-B, and as described in applicant's co-pending U.S. patent application Ser. No. 14/515,324 , entitled “Tissue Expansion Devices, Systems and Methods”, filed Oct. 15, 2014, the content of which is incorporated herein by reference in its entirety for all purposes. Tissue capture chamber 510 comprises a cylindrical structure with an opening 512 positioned in the top surface of tissue capture chamber 510 (e.g. the top surface being opposite a bottom surface that is oriented toward shaft 401 and attached to expandable element 530 of functional assembly 500, as shown in FIGS. 7A-B). Tissue capture chamber 510 can comprise the distal portion of conduit 5010, as shown. Alternatively, tissue capture chamber 510 can comprise a discrete piece, such as an injection molded piece, operably attached to conduit 5010 (e.g. one or more lumens of chamber 510 are fluidly and/or otherwise relatively continuously attached to one or more corresponding lumens of conduit 5010).

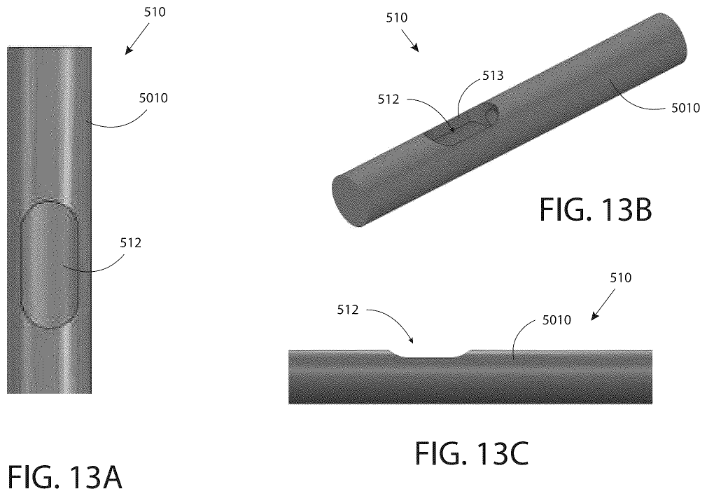

Referring specifically to FIG. 12A, opening 512 can comprise a width W1 that is less than or equal to 2.0 mm, such as a width of approximately 1.5 mm. Opening 512 can comprise a length L1 that is less than or equal to 5.0 mm, such as a length of approximately 4.0 mm. In some embodiments, opening 512 is encompassed by one or more upward facing flat portions, flat 514, comprising a portion of the wall of tissue capture chamber 510 surrounding opening 512 (e.g. flat portions created during a skiving or other procedure for creating opening 512 in chamber 510). Flat 514 can extend from the surface of chamber 510 at an angle between 90° and 175°, such as an angle between 90° and 150°, such as an angle between 132.5° and 137.5°. such as at an angle of approximately 135°. Alternatively, opening 512 does not include flat 514, such as when opening 512 is created using a punch or other method leaving only vertical walls surrounding opening 512, as described in reference to FIGS. 13A-C herebelow. Referring specifically to FIG. 12B, opening 512 can comprise a depth D1 of approximately 1.4 mm. Referring specifically to FIGS. 12C and 12D, tissue capture chamber 510 can comprise lumens 5012 and 5014, such that lumen 5012 is positioned above lumen 5014 (e.g. lumens 5012 and 5014 are in a stacked arrangement). Lumens 5012 and 5014 can be constructed and arranged to terminate within or proximate opening 512. Lumen 5012 can comprise a relatively circular or other elliptical shaped cross sectional geometry, and lumen 5014 can comprise a crescent shaped cross sectional geometry, as shown. Lumen 5012 can be positioned above lumen 5014, such that the crescent shaped geometry of lumen 5014 relatively surrounds the cylindrical structure of lumen 5012. Opening 512 includes vertical side walls 513, (e.g. vertical walls created during a skiving, punch, molding, or other process for creating opening 512 in chamber 510).

FIGS. 13, 14, 15, and 16 are top, perspective, and side views, (A-C of each respectively), of various embodiments of a tissue capture chamber, consistent with the present inventive concepts. Each tissue capture chamber 510 comprises an opening 512 with side walls 513. In some embodiments, opening 512 is surrounded by one or more flat portions, flat 514.

Referring now to FIG. 13A-C, opening 512 comprises an oblong-shaped opening, as shown. In some embodiments, an oblong-shaped opening can be created using a punch. Chamber 510 of FIGS. 13A-C does not include a flat portion (e.g. flat 514) surrounding opening 512.

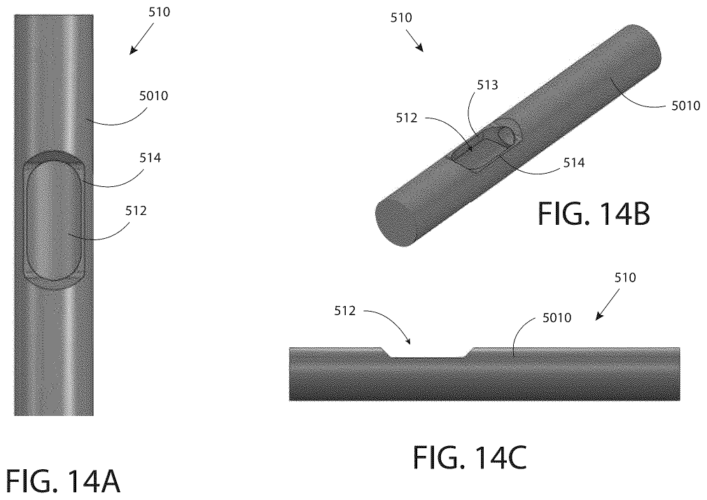

Referring now to FIG. 14A-C, opening 512 comprises a shallow skived opening, as shown, such as an opening created using a skiving procedure. Chamber 510 of FIGS. 14A-C does include a flat portion, flat 514 shown, whose width is dependent on the depth of the skive.

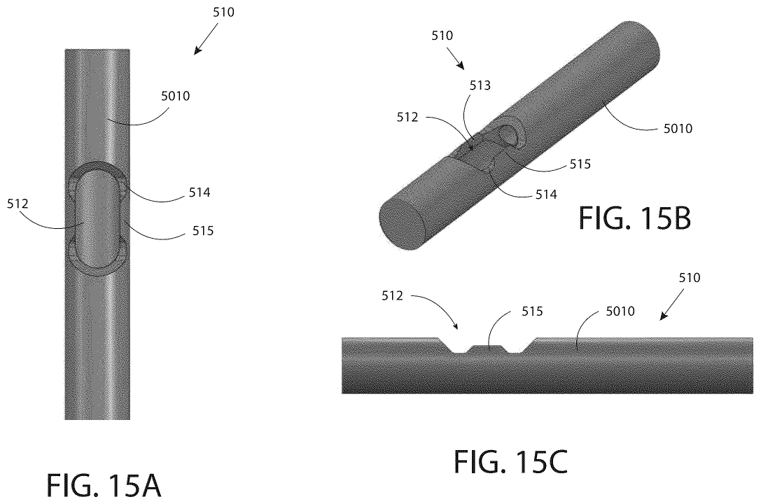

Referring now to FIG. 15A-C, opening 512 comprises one or more projections along either side of its length, projections 515, as shown. In some embodiments, projections 515 are positioned at the midpoints of each side of opening 512. Projections 515 can help prevent and/or minimize damage to the tissue (e.g. muscularis tissue of the intestine) by restricting the depth at which tissue can descend into opening 512 upon the application of a vacuum or other negative pressure to chamber 510, as described herein.

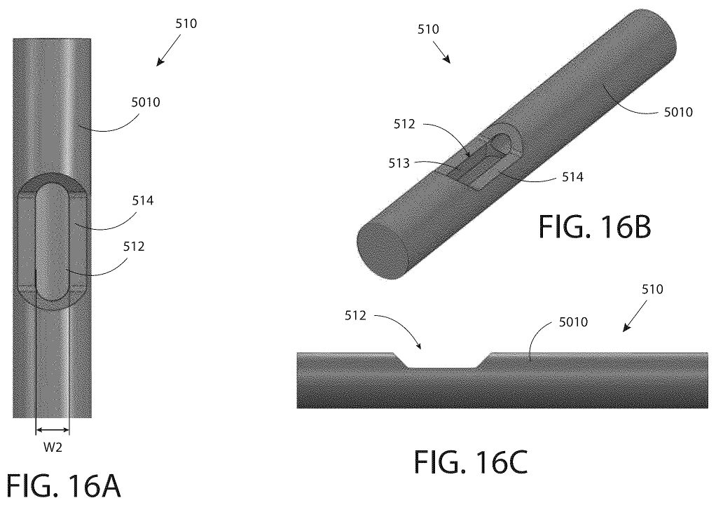

Referring now to FIG. 16A-C, opening 512 comprises a relatively narrow, oblong opening, as shown. Opening 512 can be created with a punch. The width of opening 512 can be selected to prevent and/or minimize damage to tissue (e.g. muscularis tissue of the intestine) by limiting the depth at which tissue can descend into opening 512 upon the application of a vacuum or other negative pressure, as described herein. In some embodiments, the narrow, oblong punched opening comprises a width W2 of approximately less than or equal to 2 mm, such as a width of approximately 1 mm.

Referring now to FIGS. 17A-C, side sectional views of an embodiment of a tissue capture chamber and an injectate delivery element advanced to different positions are illustrated, consistent with the present inventive concepts. Tissue capture chamber 510 comprises opening 512 and lumens 5012 and 5014. In some embodiments, a tube, such as a hypotube, sleeve 5040 shown, is positioned within the distal portion of lumen 5012. Sleeve 5040 includes a distal projection, distal stop 5041, and a proximal projection, proximal stop 5042. Distal stop 5041 and/or proximal stop 5042 can each comprise tubes (e.g. concentric hypotubes) frictionally engaged within sleeve 5040, configured to reduce the inner diameter of sleeve 5040 at distal and proximal locations as shown. Sleeve 5040 can be constructed and arranged to slidingly receive injectate delivery element 520, such as needle 525 as shown. Needle 525 is fluidly connected to conduit 521. For example, needle 525 is press fit into conduit 521. Needle 525 can comprise a diameter that ranges from 16 gauge to 34 gauge, such as a needle with a 27 gauge to 29 gauge diameter. Needle 525 can include a projection, needle ferrule 5045, such as a tube (e.g. a hypotube) frictionally engaged about a portion of needle 525. Ferrule 5045 can be slidingly received within sleeve 5040, between proximal stop 5042 and distal stop 5041. Sleeve 5040, proximal stop 5042, distal stop 5041, and ferrule 5045 can be sized such that ferrule 5045 (and therefore needle 525) slide freely proximally and distally (e.g. along the major axis of chamber 510) between proximal stop 5042 and distal stop 5041, but proximal travel is limited when ferrule 5045 makes contact with proximal stop 5042 (e.g. when needle 525 is fully retracted as shown in FIG. 17A), and distal travel is limited when ferrule 5045 makes contact with distal stop 5041 (e.g. when needle 525 is fully advanced as shown in FIG. 17B). As described in FIGS. 3, 3A, and 3B, needle 525 can be advanced and/or retracted using a control assembly, such as FDE control assembly 3100, which can also be configured to limit the force applied to needle 525 (e.g. by conduit 521 via control assembly 3100) to advance and/or retract needle 525. As described hereabove, proximal stop 5042, distal stop 5041, and ferrule 5045 are constructed and arranged to limit the distal most (retracted) and proximal most (advanced) position of needle 525. For example, FDE control assembly 3100 can exert a retraction force on conduit 521, and if needle ferrule 5045 is (already) in contact with proximal stop 5042, spring 3126 (of FIG. 3) can compress to compensate for additional retraction of knob 3114 (of FIG. 3).

In FIG. 17A, needle 525 is fully retracted within sleeve 5040, such that ferrule 5045 is in contact with proximal stop 5042 and the tip of needle 525 is positioned within distal stop 5041 (i.e. does not extend into opening 512). In this fully retracted position, vacuum can be applied to chamber 510, as described herein, causing tissue (e.g. not shown but at least mucosal and/or submucosal tissue of the intestine), to be drawn into opening 512.

In FIG. 17B, needle 525 has been partially advanced within sleeve 5040, such that the tip of needle 525 slightly extends beyond the distal end of distal stop 5041, into opening 512. In FIG. 17C, needle 525 has been fully advanced within sleeve 5040, such that the tip of needle 525 is extending into opening 512 and ferrule 545 is in contact with distal stop 5041. When needle 525 is fully advanced, the distal end of needle 525 can be relatively centered in opening 512. Needle 525 can be configured to traverse a travel length D1 of approximately 4 mm (e.g. needle 525 travels approximately 4 mm from the fully retracted position as shown in FIG. 17A to the fully advanced position as shown in FIG. 17C). In the fully advanced positioned, needle 525 can comprise an exposed length D2 of approximately 2.5 mm (e.g. the tip of needle 525 extends into opening 512 by approximately 2.5mm). In the fully advanced position, when tissue has been captured in opening 512, injectate can be delivered via needle 525 into the tissue, as described herein, such as to expand the tissue to create a restriction (e.g. a therapeutic restriction), and/or prepare the tissue (e.g. create a safety margin of tissue) for a subsequent tissue ablation procedure.

Referring now to FIG. 18, a perspective view of a skive tool is illustrated, consistent with the present inventive concepts. Tool 20 shown can be used to create an opening between the outer surface of shaft 401 and a lumen of shaft 401 (e.g. lumens 4002, 4004, 4006, 4008 and/or 4010 described herein). Tool 20 comprises a housing 21 with a lumen 22 therethrough. Housing 21 can further include a proximal end opening 23, and a recess 24 that includes an opening 25 for exposing a portion of lumen 22. In some embodiments, tool 20 further includes a skiving instrument, skiving element 26, such as a razor blade or other cutting element. In some embodiments, tool 20 further includes a mandrel 28.

Housing 21 can be configured to slidingly receive a shaft 401 via proximal end opening 23. Shaft 401 can be advanced through lumen 22 until it reaches the distal end of housing 21 (e.g. the end opposite proximal end opening 23), such that the distal end of housing 21 serves as a hard-stop (e.g. limits the travel of shaft 401 through lumen 22). Additionally or alternatively, shaft 401 can be advanced through lumen 22 until a mark, or other indicator of correct positioning is aligned with a portion of housing 21, such as within opening 25. Opening 25 provides access to shaft 401 such that a skiving operation can be performed using skiving element 26 (such as is described herebelow in reference to FIG. 20). Recess 24 can be constructed and arranged to control (e.g. determine) the size and depth of the skive(s) created in shaft 401.

In alternative embodiments, tool 20 comprises a rotatable cutting tool, such as a drill bit, that is configured to create a skive(s) in shaft 401.

Referring now to FIG. 19, a perspective view of a twisting tool is illustrated, consistent with the present inventive concepts. Twisting tool 30 comprises a distal clamp 31, a proximal clamp 34, a heat element 32 positioned on a guiderail 33. Distal clamp 31 can be configured to removably attach to shaft 401. Proximal clamp 34 can be configured to removably attach to shaft 401 and to rotate, such as to apply a twist to shaft 401. Heat element 32 can be configured to translate the length of twisting tool 30 via guiderail 33, while applying heat (e.g. air or other gas at an elevated temperature) to shaft 401 (such as is described herebelow in reference to FIG. 20). During application of heat to shaft 401, tool 30 (and shaft 401) can be positioned horizontally and/or vertically. In some embodiments, tool 30 is configured to treat multiple shafts 401 simultaneously.

Referring now to FIG. 20, a method for preparing a multi lumen shaft for construction of a catheter as described herein is illustrated, consistent with the present inventive concepts.

Method 2000 comprises skiving shaft 401 (e.g. to provide access to one or more lumens within catheter 200, such as is described hereabove in reference to FIGS. 8A-C), and/or creating a twist in shaft 401 (e.g. as described hereabove in reference to FIG. 6).

In Step 2010, a working area, including tools 20 and 30, can be prepared for clean manufacturing. One or more shafts 401 can be brought into the working area once prepared.

In Step 2020, one or more skives into shaft 401 are created. Mandrel 28 is inserted into tool 20 via proximal end opening 23 of housing 21, as described hereabove in reference to FIG. 18. Mandrel 28 can be inserted into tool 20 until it exits the distal end of housing 21 (e.g. a minimum length exits, such as approximately 3 inches). Once mandrel 28 exits the distal end of housing 21, the proximal portion of mandrel 28 is inserted into a lumen (e.g. lumen 4004, 4006, 4008, or 4010) of shaft 401. In some embodiments, a minimum length (e.g. approximately 4 inches) of mandrel 28 is inserted into the lumen to be skived. Without deforming shaft 401, shaft 401 is inserted into tool 20 until it reaches the distal end of housing 21, or until another indicator is reached, such as to longitudinally position shaft 401 relative to tool 20 to create a skive at a desired axial location of shaft 401. In order to achieve proper rotational positioning of shaft 401, a radial marker (e.g. a marker in line with the desired lumen to be skived, such as marker 402 described herein) is positioned such that the indicator is properly positioned within opening 25 (e.g. centered in opening 25). Subsequently, skiving element 26 (e.g. a razor blade) is used to gently skive shaft 401 via opening 25, such as in a distal to proximal direction. Subsequent skives with element 26 can be performed, such as to remove burrs. In some embodiments, a skiving element 26 is replaced with a new skiving element, for example after a single or limited number of skiving operations are performed. After the skiving operation is complete, shaft 401 can be removed from tool 20 via proximal end opening 23, and a visual inspection can be conducted to verify there has been no damage to adjacent lumens and/or to verify the correct lumen has been cut. Shaft 401 can be reinserted into tool 20 (e.g. if removed for inspection after a skiving operation), such as to repeat step 2020 to perform multiple skiving operations to expose multiple lumens of shaft 401 (e.g. two or more of lumens 4004, 4006, 4008, and/or 4010).

In Step 2030, a twist can be set (e.g. heat set) into shaft 401. Twisting tool 30 can configured with the following settings: a hotbox air temperature of between 100° F. and 1000° F., such as between 310° F. and 330° F.; an axial travel speed of between 3 mm/s and 7mm/s, such as 5.5 mm/s; a hotbox airflow of between 5 scfm and 50 scfm, such as approximately 34 scfm; a segment of shaft 401 of approximately 7 inches from the distal end that is not twisted; a pneumatic jaw pressure of approximately 90 psi; and/or a pre-twist of shaft 401 of approximately 1080° (e.g. between 3 and 4 full counterclockwise twists). Shaft 401 is inserted into twisting tool 30. The distal end of shaft 401 is clamped into distal clamp 31, such that a pre-determined length (e.g. approximately 7″) of shaft 401 extends beyond distal clamp 31. This pre-determined distal segment of shaft 401 is excluded from subsequent heating and twisting, such as to prevent undesired twisting to the distal end of shaft 401. Distal clamp 31 is located proximate heat element 32. A marker, such as marker 302 (e.g. a black stripe denoting guidewire lumen 4010 and/or another lumen of shaft 401) can be oriented relative to tool 30 (e.g. oriented to point straight up). At proximal clamp 34, the proximal end of shaft 401 is twisted a desired amount (e.g. a twist of approximately 1080°). A moderate tension can be applied to shaft 401, such as to prevent shaft 401 from sagging between clamps 31 and 34. A visual inspection can be conducted to ensure the desired twist is present in shaft 401 between clamps 31 and 34. Heat is then applied to the twisted portion of shaft 401. In some embodiments, heating element 32 translates along guiderail 33, applying the prescribed heat to shaft 401, at the prescribed rate (as described hereabove). Alternatively or additionally, shaft 401 can be translated about heating element 32 (e.g. with or without translation of heating element 32). After sufficient heating, and after shaft 401 has cooled, shaft 401 can be removed from twisting tool 30.

In Step 2040, an optional step of measuring shaft 401 to ensure the proper twist has been applied can be performed. Twist measurements can be made at least 24 hours after the application of heat performed in Step 2030, such as to accommodate relaxation of shaft 401 that occurs over time. A tape measure or other measuring tool (e.g. a measuring device that is greater in length than shaft 401) can be fixed to a flat surface (e.g. taped or otherwise attached to a flat work bench). Shaft 401 can be positioned parallel to the tape measure and the distal end of shaft 401 can be manually rotated until the portion of the marker (e.g. marker 302) at the distal end of shaft 401 is oriented (e.g. to point straight up). The distance from the distal end of shaft 401 to the first full rotation (e.g. where the marker is also pointing straight up) can measure approximately 18.5 inches.

Referring now to FIG. 21, a perspective view of an example handle portion of catheter 200 including a tactile thermal status indicator are illustrated, consistent with the present inventive concepts. Handle assembly 300 comprises housing 301 which surrounds various mechanisms and other assemblies of handle assembly 300, such as described hereabove. In some embodiments, and as shown in FIG. 21, inflow conduit 3204 can be routed, or otherwise positioned, within handle 300 proximate a thermally conductive housing portion, portion 303. At least a portion of inflow conduit 3204 can be constructed and arranged to readily transfer thermal energy between conduit 3204 and portion 303 (e.g. at least a portion of conduit 3204 can comprise a thin walled portion, can comprise a thermally conductive material such as a metal, and/or it can comprise a conduit portion integral to portion 303), such that an operator of catheter 200 can detect, or otherwise notice, the relative temperature of the fluid within inflow conduit 3204. For example, during a heat ablation procedure, portion 303 is constructed and arranged to heat up, allowing the operator of catheter 200 to confirm hot ablative fluid is flowing to expandable element 530 (as described herein).

Additionally or alternatively, during a neutralizing procedure step, portion 303 can be constructed and arranged to cool down, allowing the operator to confirm cold neutralizing fluid is present within expandable element 530 (as described herein). In cryogenic procedures, cooling of portion 303 can indicate cryogenic ablation is occurring (e.g. cryogenic fluid is present within expandable element 530) while warming of portion 303 can indicate a neutralizing step is in process (e.g. warm neutralizing fluid is within expandable element 530). In some embodiments, a functional element of handle 300 (such as functional element 399 described hereabove) can comprise a heating or cooling transducer configured to provide real-time information (e.g. real-time temperature information, pressure information, and/or other feedback) to an operator of catheter 200, which provides feedback relative to the temperature, pressure and/or other functional real-time characteristic of functional assembly 300 or other component of system 10.

While ablation catheter 200, console 100 and other components of system 10 of the present inventive concepts have been described for use in the intestine, it should be appreciated that use in other portions of the GI tract and other portions of mammalian anatomy should be considered within the spirit and scope of this application. For example, expansion of tissue and subsequent ablation of tissue can similarly be performed in the esophagus, stomach, or colon of a patient.

While the preferred embodiments of the devices and methods have been described in reference to the environment in which they were developed, they are merely illustrative of the principles of the inventions. Modification or combinations of the above-described assemblies, other embodiments, configurations, and methods for carrying out the invention, and variations of aspects of the invention that are obvious to those of skill in the art are intended to be within the scope of the claims. In addition, where this application has listed the steps of a method or procedure in a specific order, it may be possible, or even expedient in certain circumstances, to change the order in which some steps are performed, and it is intended that the particular steps of the method or procedure claim set forth below not be construed as being order-specific unless such order specificity is expressly stated in the claim.

1-59. (canceled)

60. A method of treating mucosal tissue in a duodenum of a patient, the method comprising:
introducing into the duodenum of the patient a catheter comprising an elongate shaft and an expandable functional assembly disposed on a distal portion of the elongate shaft, the expandable functional assembly comprising:
(i) at least one expandable reservoir configured to receive a heated liquid for hydrothermal ablation of mucosal tissue,
(ii) a plurality of tissue capture chambers disposed on the expandable functional assembly, and
(iii) a plurality of injectate delivery elements respectively associated with the plurality of tissue capture chambers;
positioning the expandable functional assembly at a first target location in the duodenum distal to an ampulla of Vater;
applying vacuum to the plurality of tissue capture chambers and delivering injectate through the plurality of injectate delivery elements to expand submucosal tissue at the first target location;
under direct endoscopic visualization, determining whether the expanded submucosal tissue at the first target location is adequate for ablation; and
responsive to determining that the expanded submucosal tissue is adequate, delivering the heated liquid to the at least one expandable reservoir to hydrothermally ablate mucosal tissue at the first target location with the expandable functional assembly, wherein the hydrothermal ablation is performed without repositioning the expandable functional assembly after the expanding of the submucosal tissue.

61. The method of claim 60, wherein determining whether the expanded submucosal tissue is adequate comprises at least partially collapsing the expandable functional assembly to improve an endoscopic view of the expanded submucosal tissue.

62. The method of claim 61, wherein the at least partially collapsed expandable functional assembly permits advancement of an endoscope toward the first target location to obtain a closer view of the expanded submucosal tissue.

63. The method of claim 60, wherein the first target location is at least 1 cm distal to the ampulla of Vater.

64. The method of claim 60, further comprising translating the catheter to a second target location distal to the first target location and repeating the applying, determining, and hydrothermally ablating while maintaining a papilla exclusion zone.

65. The method of claim 60, further comprising, before the hydrothermally ablating, delivering injectate to expand submucosal tissue at a second axial location adjacent the first target location.

66. The method of claim 65, wherein submucosal tissue expanded at the first target location and the second axial location together defines an expanded tissue periphery that sufficiently surrounds an ablation periphery created during the hydrothermally ablating.

67. The method of claim 60, wherein the plurality of tissue capture chambers comprises three tissue capture chambers circumferentially arranged about the at least one expandable reservoir.

68. The method of claim 60, wherein each injectate delivery element comprises a needle positioned above a source of vacuum provided to an associated tissue capture chamber, each needle having a diameter between 16 gauge and 34 gauge and being advanced at least 2.5 mm into tissue captured by the associated tissue capture chamber.

69. The method of claim 60, wherein the injectate comprises a visible material or a radiopaque material to assist in assessing adequacy of the expanded submucosal tissue.

70. An ablation catheter for treating mucosal tissue in a duodenum of a patient, the ablation catheter comprising:
an elongate shaft having a distal portion;
an expandable functional assembly disposed on the distal portion and comprising at least one expandable reservoir configured to receive a heated liquid for hydrothermal ablation of mucosal tissue;
at least two tissue capture chambers disposed on the expandable functional assembly, each tissue capture chamber being configured to capture tissue upon application of vacuum;
at least two injectate delivery elements respectively associated with the at least two tissue capture chambers and configured to deliver injectate into tissue captured by the at least two tissue capture chambers;
at least two vacuum delivery conduits respectively fluidly connected to the at least two tissue capture chambers;
at least two injectate delivery conduits respectively fluidly connected to the at least two injectate delivery elements;
a first fluid conduit fluidly connected to the at least one expandable reservoir and configured to deliver fluid to the at least one expandable reservoir; and
a second fluid conduit fluidly connected to the at least one expandable reservoir and configured to remove fluid from the at least one expandable reservoir,
wherein the at least two tissue capture chambers, the at least two injectate delivery elements, and the at least one expandable reservoir are co-located on the expandable functional assembly such that the ablation catheter is configured to expand submucosal tissue and thereafter hydrothermally ablate mucosal tissue at a same axial location without repositioning the expandable functional assembly.

71. The ablation catheter of claim 70, wherein the at least two tissue capture chambers comprise three tissue capture chambers circumferentially arranged about the at least one expandable reservoir.

72. The ablation catheter of claim 70, wherein each injectate delivery element is positioned above a source of vacuum provided to an associated tissue capture chamber.

73. The ablation catheter of claim 70, wherein each tissue capture chamber comprises a first lumen and a second lumen, the second lumen being positioned above the first lumen, the first lumen having a crescent-shaped cross-sectional geometry and the second lumen having a cylindrical geometry.

74. The ablation catheter of claim 70, wherein each injectate delivery element comprises a needle.

75. The ablation catheter of claim 74, wherein each needle has a diameter between 16 gauge and 34 gauge.

76. The ablation catheter of claim 75, wherein each needle has a bevel angle of between 5° and 45° and is configured to be advanced at least 2.5 mm into tissue captured within an associated tissue capture chamber.

77. The ablation catheter of claim 70, wherein the expandable functional assembly is configured to at least partially collapse prior to and/or during an endoscopic assessment of submucosal tissue expansion to provide an increased view of expanded submucosal tissue.

78. The ablation catheter of claim 70, wherein the elongate shaft comprises at least six lumens, including a first pair of lumens in fluid communication with a first tissue capture chamber, a second pair of lumens in fluid communication with a second tissue capture chamber, and a third pair of lumens in fluid communication with the at least one expandable reservoir.

79. The ablation catheter of claim 78, wherein the third pair of lumens comprises a fluid delivery lumen configured to deliver fluid to the at least one expandable reservoir and a fluid removal lumen configured to remove fluid from the at least one expandable reservoir.
